(19) United States
(12) Patent Application Publication
(10) Pub. No.: US 2015/0284673 A1
(43) Pub. Date: Oct. 8, 2015

(54) HARVESTING ALGAE FROM WATER

(71) Applicant: SAPPHIRE ENERGY, INC., San Diego, CA (US)

(72) Inventors: Timothy J. Langer, Los Angeles, CA (US); Shinsyu Matsumoto, Las Cruces, NM (US); Alex M. Aravanis, Foster City, CA (US)

(73) Assignee: SAPPHIRE ENERGY, INC., SAN DIEGO, CA (US)

(21) Appl. No.: 14/649,524

(22) PCT Filed: Nov. 25, 2013
(86) PCT No.: PCT/US13/71633

§ 371 Date: Jun. 3, 2015

Related U.S. Application Data
(60) Provisional application No. 61/736,939, filed on Dec. 13, 2012.

(51) Int. Cl. C12N 1/12

(52) U.S. Cl. CPC C12N 1/12

(57) ABSTRACT

The present application includes methods to harvest a non-vascular photosynthetic organism (NVPO) such as microalgae from an aqueous culture comprising brackish, non-brackish, marine, sea or saline water using polymer flocculants. The methods are suitable for harvesting NVPO from aqueous culture with total dissovled solids (TDS) of at least 1500 mg/L. Methods are also provided to harvest a NVPO using flocculation with or without a Dissolved Air Flotation (DAF) process. Methods are further provided to flocculate and harvest a NVPO directly in a pond. The present application further provides NVPO-containing intermediates, compositions, or products produced by the methods provided herein.

### FIELD OF THE INVENTION

The present disclosure includes methods of harvesting a non-vascular photosynthetic organism (NVPO) from an aqueous culture by flocculation and NVPO-comprising products derived from such methods.

### BACKGROUND OF THE INVENTION

There is a pressing need to develop alternative fuel sources that are renewable, sustainable, and less harmful to the environment. Biofuel produced from algae represents one such alternative source.

Algae are non-vascular photosynthetic organisms (NVPOs), producing oxygen by photosynthesis. One group, the microalgae, is useful for biotechnology applications for many reasons, including their high growth rate and tolerance to varying environmental conditions. The use of microalgae in a variety of industrial processes for commercially important products is known. For example, microalgae have uses in the production of nutritional supplements, pharmaceuticals, natural dyes, a food source for fish and crustaceans, biological control of agricultural pests, production of oxygen and removal of nitrogen, phosphorus and toxic substances in sewage treatment, and pollution controls, such as biodegradation of plastics or uptake of carbon dioxide.

Microalgae, like other organisms, contain lipids and fatty acids as membrane components, storage products, metabolites and sources of energy. Some algal strains, diatoms, and cyanobacteria have been found to contain proportionally high levels of lipids (over 30%). Microalgal strains with high oil or lipid content are of interest to the development of a sustainable feedstock for the production of biofuel.

Microalgae can produce 10 to 100 times as much mass as terrestrial plants in a year. Such microalgae can grow almost anywhere, though are most commonly found at latitudes between 40 N and 40 S. With more than 100,000 known species of diatoms (a type of microalgae), 40,000 known species of green plant-like microalgae, and smaller numbers of other microalgae species, microalgae will grow rapidly in nearly any environment, with almost any kind of water, including marginal areas with limited or poor quality water.

Algae are typically grown in liquid environments. It is sometimes beneficial to harvest algae from the liquid environments for further processing. Harvesting of algae requires one or more solid-liquid separation steps, and represents a significant challenge because of the typically small size and low concentration of algae. Any suitable harvest method must be able to process the large volumes typical of algal biomass production processes.

Algae may be harvested by various methods including centrifugation, filtration, gravity sedimentation, and combinations thereof. One method is to flocculate or aggregate algae to facilitate harvest. Flocculants or flocculating agents promote flocculation by causing colloids and other suspended particles (e.g., cells) in liquids to aggregate and form a floc. Flocculants are used in water treatment processes to improve the sedimentation of small particles. For example, a flocculant may be used in swimming pool or drinking water filtration to aid removal of microscopic particles which would otherwise cause the water to be cloudy and which would be difficult to remove by filtration alone.

Many flocculants are multivalent cations such as aluminium, iron, calcium, or magnesium. These positively charged molecules interact with negatively charged particles and molecules to reduce the barriers to aggregation. In addition, many of these chemicals, under appropriate pH and other conditions such as temperature and salinity, react with water to form insoluble hydroxides which, upon precipitating, link together to form long chains or meshes, physically trapping small particles into larger flocs.

Microalgal cells carry a negative surface charge that can prevent aggregation of cells in suspension. The intensity of this negative surface charge is a function of algal species, medium ionic strength, pH, and other environmental conditions. Sources of the algal surface electric charge include ionization of ionogenic functional groups at the algal cell wall and selective adsorption of ions from the culture medium. The algal cell surface charge can be neutralized or reduced by adding flocculants such as multivalent cations and cationic polymers to the medium. The closer the net surface charge of the algae is to zero the more likely they are to aggregate. Ideally, the flocculants used should be inexpensive, nontoxic, and effective in low concentration. In addition, the flocculant should be selected so that further downstream processing is not adversely affected by its use.

Organic cationic polymers, such as modified polyacrylamides, may induce flocculation of freshwater microalgae at low dosages (between 1 and 10 ppm). However, the high salinity of the marine environments inhibits algae flocculation with polymers. Marine and brackish waters may contain total dissolved solids (TDS) up to approximately 50000 mg/L. This high TDS level may interfere with the configuration and dimension of polymers. At a high TDS environment, polymers shrink to their smallest dimensions, and fail to bridge between algal cells. Of particular need is an efficient method to harvest algae from salt solutions such as brackish or marine waters.

### SUMMARY OF THE INVENTION

The present disclosure includes a method of harvesting a non-vascular photosynthetic organism (NVPO) from an aqueous culture by mixing an effective amount of a polymer flocculant with the aqueous culture to form flocs of the NVPO, introducing dissolved air into the mixed aqueous culture, and collecting the flocs of the NVPO.

The present disclosure includes a method of harvesting a non-vascular photosynthetic organism (NVPO) from an aqueous culture by mixing an effective amount of a polymer flocculant with the aqueous culture to form flocs of the NVPO, and collecting the floes of the NVPO, wherein the method does not involve dissolved air flotation.

The present disclosure includes a method of harvesting a non-vascular photosynthetic organism (NVPO) from an aqueous culture by mixing an effective amount of a polymer flocculant and/or a coagulant with the aqueous culture to form flocs and/or increase the density of the NVPO, and collecting the floes of the NVPO, wherein the method is performed using a clarifier.

The present disclosure includes a method of harvesting a NVPO from an aqueous culture of the NVPO by mixing an effective amount of a flocculant with the aqueous culture in an open pond to form floes of the NVPO; and collecting the floes of the NVPO, wherein the method is performed in a pond.

A method of the present disclosure can be used to harvest an NVPO from an aqueous culture comprising a total dissolved solids (TDS) of any level. In one aspect, a method of the present disclosure may be used to harvest an NVPO from brackish water having a TDS selected from the group consisting of between 1500 and 35000 mg/L.

The present disclosure includes an aqueous culture comprising a non-vascular photosynthetic organism (NVPO) at a concentration between 0.001% and 0.2%, and a polymer flocculant at a concentration of less than 10 parts per million (ppm).

The present disclosure includes an aqueous culture having a TDS between 1500 and 35000 mg/L and comprising a non-vascular photosynthetic organism (NVPO) at a concentration between 0.001% and 0.2%, and a polymer flocculant at a concentration of less than 10 parts per million (ppm).

The present disclosure includes a biomass slurry comprising a non-vascular photosynthetic organism (NVPO) at a concentration of at least 0.2% and a polymer flocculant, wherein the ratio between the weight of the polymer flocculant and the Ash-Free Dry Weight (AFDW) of the NVPO is at most 1.5%.

The present disclosure includes a biomass slurry having a TDS between 1500 and 35000 mg/L and comprising a non-vascular photosynthetic organism (NVPO) at a concentration of at least 0.2% and a polymer flocculant, wherein the ratio between the weight of the polymer flocculant and the Ash-Free Dry Weight (AFDW) of the NVPO is at most 1.5%.

The present disclosure includes an aqueous medium comprising a non-vascular photosynthetic organism (NVPO) at a concentration between 0.0001% and 0.02%, and a polymer flocculant at a concentration of less than 1 part per million (ppm).

The present disclosure includes an aqueous medium having a TDS between 1500 and 35000 mg/L and comprising a non-vascular photosynthetic organism (NVPO) at a concentration between 0.0001% and 0.02%, and a polymer flocculant at a concentration of less than 1 parts per million (ppm).

### BRIEF DESCRIPTION OF THE DRAWINGS

FIG. 1: A diagram of a typical DAF process.

FIG. 2: Analysis of harvested solids content from in-pond polymer-assisted harvest.

FIG. 3: Analysis of harvested volume from in-pond polymer-assisted harvest.

FIG. 4: Analysis of harvested solids from in-pond polymer-assisted harvest.

FIG. 5: Analysis of polymer/algae ratios from in-pond polymer-assisted harvest.

FIG. 6: Effects of in-pond addition of polymers on strain performance measured by Microwave Dry weight (M.W.D.W.), an equivalent metric for total suspended solids (TDS).

FIG. 7: Effects of in-pond addition of polymers on strain performance measured by the ratio of variable to maximum fluorescence (FV/FM), an indicator of algal nutrient status.

FIG. 8: In-pond addition of polymers for harvesting has no significant effects on the average daily algal production. One-way ANOVA analysis is shown for in-pond harvests using a 0.25% 895BS stock solution (In-pond 0.25% Treatment) or using a 0.5% 895BS stock solution (In-pond 0.50%) and a control harvest using a standard DAF process (DAF). Ten, nine, and nine harvests are performed for the control, In-pond 0.25%, and In-pond 0.50% treatments, respectively.

FIG. 9: Effects of in-pond addition of polymers on the nitrate level of an aqueous culture.

FIG. 10: Effects of in-pond addition of polymers on the phosphate level of an aqueous culture.

FIG. 11: Dose response of Monolyte amine polymer CE-3990 for strains SE0004 and SE0086.

FIG. 12: Higher algae density requires higher concentrations of polymers for effective flocculation.

FIG. 13: Dosing recommendations of Nalco polymer GR-505 on strain SE0004.

FIG. 14: A comparison of flocculation effectiveness of Nalco polymer GR-505 and Ashland polymers K290FL and FLX with strain SE0087 in MASM 16 ppt media.

FIG. 15: A comparison of flocculation effectiveness of Nalco non-GRAS polymers and Ashland 859BS with strain SE0087 in MASM 16 ppt media.

FIG. 16: Time-dependent degradation of a 0.25% GR-505 stock solution.

FIG. 17: Effectiveness of GR-505 to flocculate strain SE0087 in a wide pH range.

FIG. 18: Induction of pH change by GR-505 when flocculating strain SE0087 in MASM media.

FIG. 19: A comparison of flocculation effectiveness of Nalco GR-505 and Ashland emulsion polymers with strain SE0087 in MASM 16 ppt media.

FIG. 20: A comparison of flocculation effectiveness of Nalco polymers compared to Ashland cationic emulsions with strain SE0087 in IABR 10 ppt media.

FIG. 21: Dose response of coagulants with strain SE0087 in MASM.

FIG. 22: Achieved efficiencies using Ashland cationic emulsion polymers with strain SE0087 in MASM 16 ppt media.

FIG. 23: Mixing effects on polymer effectiveness.

FIG. 24: Salt effects on performance of cationic polymers with strain SE0004 in G-media.

FIG. 25: Salt effects on performance of cationic polymers with strain SE0087 in MASM media.

FIG. 26: Dose response of 859BS based on culture density of strain SE0087 in MASM 16 ppt media.

FIG. 27: Dose response for 189K and 859BS combination with strain SE0087 in MASM media.

FIG. 28: Minimal degradation of a 859BS stock solution over time.

FIG. 29: Effectiveness of 859BS to flocculate strain SE0087 in a wide pH range.

FIG. 30: Induction of pH change by 859BS when flocculating strain SE0087 in MASM media.

FIG. 31: Impacts of Alkalinity on flocculation effectiveness of 859BS with strain SE0087 in MASM media.

FIG. 32: Single data series from a calibration of PVSAK in filtered MASM.

FIG. 33: Calibration curve of PVSAK to cationic polymer in MASM 16 ppt media.

FIG. 34: Concentration of residual 895BS at different flocculation efficiencies.

FIG. 35: Concentration of residual 895BS at various dosing and flocculation efficiencies.

FIG. 36: One-way ANOVA analysis of polymer dosing for Scenedesmus and Spirulina.

FIG. 37: One-way ANOVA analysis of Turbidity Efficiency (%) for Scenedesmus and Spirulina.

FIG. 38: One-way ANOVA analysis of the TDS of aqueous cultures for Scenedesmus and Spirulina.

FIG. 39: A process diagram for making stock solutions of a dry polymer using a 180 gallon make-down system as well as calibration.

FIG. 40: A process diagram describing procedures for preparing a manual batch of polymer flocculant stock solution used in ajar-test setting.

FIG. 41: A process diagram describing procedures for using jar-tests to determine optimal polymer dosing used for biomass harvesting by a DAF unit.

FIG. 42: A process diagram describing procedures for operating a 40 gpm WWW Dissolved Air Flotation (DAF) system to harvest algae from a pond.

FIG. 43: A process diagram describing procedures for operating a Westech Dissolved Air Flotation (DAF) unit to harvest algal biomass from a pond.

FIG. 44: A process diagram describing procedures for start-up and operation of a decanter to further dewater a biomass slurry harvested by a DAF process.

FIG. 45: A process diagram describing procedures for sample collection during a harvest of algae from a pond by a DAF process.

FIG. 46: A process diagram describing procedures for measuring moisture content of a sample using a moisture analyzer.

FIG. 47: A process diagram describing procedures for sanitizing a DAF unit.

### DETAILED DESCRIPTION

Unless defined otherwise, technical and scientific terms used herein have the same meaning as commonly understood by one of ordinary skill in the art. One skilled in the art will recognize many methods can be used in the practice of the present invention. Indeed, the present invention is in no way limited to the methods and materials described. For purposes of the present disclosure, the following terms are defined below.

The present disclosure includes a method of harvesting a non-vascular photosynthetic organism (NVPO) from an aqueous culture by mixing an effective amount of a polymer flocculant with the aqueous culture to form flocs of the NVPO, introducing dissolved air into the mixed aqueous culture, and collecting the flocs of the NVPO.

As used herein, “harvesting” relates to the removal, concentration, or isolation of all, or part of NVPOs in a culture system, including a liquid culture system. Harvesting may occur continuously from a growing culture, batchwise or as a total collection of the microalgae at the end of a culture period. “Continuous harvesting” relates to non-stop, repeated harvesting of a NVPO from an aqueous culture in which growth and harvesting of the NVPO is maintained at a relatively steady rate. “Batchwise” relates processing of an NVPO-containing culture as batches or lots in which the whole of each batch/lot is subjected to one stage of the process at a time. Upon harvesting of a NVPO, the resulting aqueous medium, as a subnatant, supernatant, flow-through or other liquid form, may be recycled or returned to an algal culture system to support the further growth of algae. Supernatant relates to a liquid which remains above the solid produced by a flocculation or coagulation process. Subnatant relates to a liquid lying under a supernatant or under flocculated or coagulated solid material. Flow-through relates to a continuously flowing stream.

In one aspect, a method of the present disclosure is used to harvest a prokaryotic NVPO. In another aspect, a method of the present disclosure is used to harvest a eukaryotic NVPO. In one aspect, a method of the present disclosure is used to harvest a filamentous NVPO. In another aspect, a method of the present disclosure is used to harvest a NVPO from a genus selected from the group consisting of Chlamydomonas, Nannochloropsis, Scenedesmus, Desmodesmus, Tetraselmis, and Arthrospira. In one aspect, a method of the present disclosure is used to harvest a unicellular NVPO. In another aspect, a method of the present disclosure is used to harvest a multicellular NVPO. In one aspect, a method of the present disclosure is used to harvest a genetically modified NVPO. In another aspect, a method of the present disclosure is used to harvest a NVPO from a monoculture. In yet another aspect, a method of the present disclosure is used to harvest a NVPO from culture comprising multiple NVPO species.

A NVPO of the present disclosure can be transgenic, selected for or naturally occurring. A NVPO of the present disclosure can be one that naturally photosynthesizes (has a plastid) or that is genetically engineered or otherwise modified to be photosynthetic. In one aspect, the photosynthetic organism can be transgenic and comprises a nucleic acid which renders all or part of the photosynthetic apparatus inoperable.

Examples of some prokaryotic organisms of the present disclosure include, but are not limited to, cyanobacteria (e.g., Synechococcus, Synechocystis, Athivspira, Gleocapsa, Oscillatoria, Leptolyngbya, and Pseudoanabaena). In one aspect, the organism is a eukaryotic alga (e.g. green algae, red algae, or brown algae). In one aspect, the alga is a green alga, for example a Chlorophycean. The alga can be a unicellular or multicellular algae. In another aspect, the host cell is a microalga (e.g., Chlamydomonas reinhardii, Dunaliella sallna, Haematococcus pluvialis, Scenedesmus dimorphus, Chlorella spp., Tetraselmis spp, Dunaliella viridis, or Dunaliella tertiolecta).

Non-limiting examples of non-vascular photosynthetic microorganisms include algae (e.g. red algae, green algae), protists (such as euglena), and bacteria (such as cyanobacteria). In one aspect, the alga is Chlamydomonas, Scenedesmus, Tetraselmis, Desmodesmus, Chlorella or Nannochlorpis. In another aspect, the alga is C. reinhardtii. In yet another aspect, the alga is C. reinhardtii 137c. Additional common non-limiting examples of non-vascular photosynthetic organisms (NVPO) that can be used with the methods of the present disclosure are members of one of the following divisions: chlorophyta, cyanophyta (cyanobacteria), and heterokontophyta, bacillariophyta, chrysophyta and haptophyta. In some instances, the microalgae are, for example, an organism classified as prochlorophyta, rhodophyta, tribophyta, glaucophyta, chlorarachniophytes, euglenophyta, euglenoids, cryptophyta, cryptomonads, dinophyta, dinoflagellata, pyrmnesiophyta, bacillariophyta, xanthophyta, eustigmatophyta, raphidophyta, phaeophyta, and phytoplankton.

Specific non-limiting examples of chlorophytes include Ankistrodesmus, Botryococcus, Chlorella, Chlorococcum, Dunaliella, Monoraphidium, Oocystis, Desmodesmus, Scenedesmus, and Tetraselmis. In one aspect, the chlorophytes can be Chlorella or Dunaliella. Specific non-limiting examples of cyanophytes include Oscillatoria, Arthrospira and Synechococcus. Specific non-limiting examples of species from the genus of Arthrospira include Arthrospira balkrishnaii, Arthrospira desikacharyensis, Arthrospira gigantean, Arthrospira gomontiana, Arthrospira jenneri, Arthrospira masartii, Arthrospira pellucidis, Arthrospira platensis, Arthrospira santannae, Arthrospira skujae, Arthrospira tenuis, Arthrospira argentina, Arthrospira fusiformis, Arthrospira ghannae, Arthrospira indica, Arthrospira joshii, Arthrospira maxima, Arthrospira amethystine, Arthrospira ardissonii, Arthrospira margaritae, Arthrospira miniata, Oscillatoria spirulinoides, Spirulina aeruginea, Spirulina allansonii, Spirulina attenuate, Spirulina cabrerae, Spirulina cavanillesiana, Spirulina erdosensis, Spirulina gordiana, and Spirulina mariae. Specific example of chrysophytes includes Boekelovia. Specific non-limiting examples of haptophytes include Isochrysis and Pleurochrysis. Specific non-limiting examples of bacillariophytes include the genera Amphipleura, Amphora, Chaetoceros, Cyclotella, Cymbella, Fragilaria, Hantzschia, Navicula, Nitzschia, Phaeodactylum, and Thalassiosira.

In certain aspects, the NVPO used with the methods of the disclosure are members of one of the following genera: Nannochloropsis, Chlorella, Dunaliella, Desmodesmus, Scenedesmus, Selenastrum, Oscillatoria, Phormidium, Spirulina, Amphora, Tetraselmis and Ochromonas.

Non-limiting examples of NVPO species that can be used with the methods of the present disclosure include: Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella Candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis alacris, Tetraselmis apiculata, Tetraselmis ascus, Tetraselmis astigmatica, Tetraselmis chuii, Tetraselmis convolutae, Tetraselmis cordiformis, Tetraselmis deskacharyi, Tetraselmis gracilis, Tetraselmis hazeni, Tetraselmis impellucida, Tetraselmis inconspicua, Tetraselmis levis, Tetraselmis maculate, Tetraselmis marina. Tetraselmis micropapillata, Tetraselmis rubens, Tetraselmis striata, Tetraselmis suecica, Tetraselmis tetrabrachia, Tetraselmis tetrathele, Tetraselmis verrucosa, Tetraselmis wettsteinii, Thalassiosira weissflogii, and Viridiella fridericiana.

Examples of non-vascular photosynthetic organisms are C. reinhardii, D. salina, D. tertiolecta, S. dimorphus, or H. pluvialis. The organism can be a member of the genus Chlamydomonas, Dunaliella, Desmodesmus, Scenedesmus or Hematococcus, for example C. reinhardtii, D. salina, D. tertiolecta, S. dimorphus or H. pluvialis, although members of other genera may be used.

In another aspect, microalgae of the present disclosure include members of the phyla heterokontophyta. In an aspect, a microalga of the phyla heterokontophyta may be member of the genus Nannochloropsis. In another aspect, a Nannochloropsis may be transgenic. In an aspect, a microalga of the present disclosure may be a microalga of the chorophyta division of the protist kingdom.

In another aspect, microalgae of the present disclosure may be a cyanobacterium. In an aspect, a cyanobacterium may be a member of the genus Spirulina, or of the genus Leptolyngbya.

In an aspect, microalgae of the present disclosure may be transgenic. In an aspect, the microalgae of the present disclosure may be transgenic according to the methods of International Patent Application No. PCT/US2010/048828, published as International Patent Publication WO 2011/034863, or according to the methods provided in International Patent Application No. PCT/US2010/048666, published as International Publication No. WO 2011/034823, both of which are hereby incorporated by reference in their entireties.

As used herein, NVPO strain “SE0004” relates to a Scenedesmus, while NVPO strain “SE0087” relates to a Nannochloropsis.

A method of the present disclosure may be used to harvest a non-vascular photosynthetic organism (NVPO) from an aqueous culture from any source. As used herein, an “aqueous culture” relates to a water-based culture comprising an NVPO of interest. An aqueous culture may be derived from a closed or an open culture system. An open culture system may include, for example an open photobioreactor, semi-closed ponds, open ponds, natural ponds, lakes, or artificial ponds.

A method of the present disclosure may be used to harvest a NVPO from an aqueous culture with any concentration of NVPO. In an aspect, the aqueous culture of the present disclosure may have one or more NVPOs at a concentration between 0.001% and 0.2%, measured by weight/volume (Ash Free Dry Weight of the NVPO/culture volume).

In another aspect, the NVPO concentration may be selected from the group consisting of between 0.001% and 0.2%, between 0.005% and 0.2%, between 0.01% and 0.2%, between 0.02% and 0.2%, between 0.03% and 0.2%, between 0.04% and 0.2%, between 0.05% and 0.2%, between 0.06% and 0.2%, between 0.07% and 0.2%, between 0.08% and 0.2%, between 0.09% and 0.2%, between 0.1% and 0.2%, between 0.15% and 0.2%, between 0.001% and 0.15%, between 0.001% and 0.1%, between 0.001% and 0.09%, between 0.001% and 0.08%, between 0.001% and 0.07%, between 0.001% and 0.06%, between 0.001% and 0.05%, between 0.001% and 0.04%, between 0.001% and 0.03%, between 0.001% and 0.02%, between 0.001% and 0.01%, between 0.001% and 0.005%, between 0.001% and 0.002%, between 0.003% and 0.15%, between 0.005% and 0.125%, between 0.01% and 0.1%, between 0.02% and 0.08%, and between 0.04% and 0.06%, measured by weight/volume (Ash Free Dry Weight (AFDW) of the NVPO/culture volume).

As used herein, “Ash free dry weight (AFDW),” also known as volatile suspended solids (VSS), relates to an overall measure of the organic biomass produced by a culture of NVPOs. One method of measuring AFDW is to obtain a minimum of 5 mg to 10 mg of AFDW biomass by centrifuging or filtering a sufficient volume of NVPO culture, washing the pellets or the filters with distilled water (for fresh water samples) or an isotonic solution of ammonium formate (e.g. 20 g/liter for seawater samples), and then drying the pellets or filters (transferred to aluminum weighing pans) at 105° C. overnight, weighing, and then ashing in a muffle furnace at 550° C. for 15 to 20 minutes, and weighing again. The pans and filters should be washed, ashed, weighed and stored in a desiccator prior to use. All samples should be stored in desiccators after removing from ovens to allow them to cool (10 minutes minimum) or for storage prior to weighing. An analytical balance (+/−0.01 mg) is required, to allow reproducibility of within 3%. AFDW is calculated as the difference of the weight of the filters or weighing boats after drying at 105° C. and after ashing at 550° C. AFDW Data may be expressed as mg/liter AFDW and ash content as % of total dry weight. Ash free dry weight may be preferred over total dry weight measurement when the presence of mineral ash would induce a significant error in the estimation of organic content dry weight.

The concentration of NVPO in the aqueous culture of the present disclosure may also be measure by Optical Density at a wavelength depending on the species being measured. In one aspect, the aqueous culture of the present disclosure may have an OD value of between 0.01 and 2 at 750 nm. In other aspects, the aqueous culture of the present disclosure may have an OD value at 750 nm selected from the group consisting of between 0.02 and 2, between 0.03 and 2, between 0.04 and 2, between 0.05 and 2, between 0.06 and 2, between 0.07 and 2, between 0.08 and 2, between 0.09 and 2, between 0.1 and 2, between 0.15 and 2, between 0.2 and 2, between 0.25 and 2, between 0.3 and 2, between 0.35 and 2, between 0.4 and 2, between 0.45 and 2, between 0.5 and 2, between 0.55 and 2, between 0.6 and 2, between 0.7 and 2, between 0.8 and 2, between 0.9 and 2, between 1 and 2, between 1.25 and 2, between 1.5 and 2, between 1.75 and 2, 0.02 and 1.9, 0.02 and 1.8, 0.02 and 1.7, 0.02 and 1.6, 0.02 and 1.5, 0.02 and 1.4, 0.02 and 1.3, 0.02 and 1.2, 0.02 and 1.1, 0.02 and 1, 0.02 and 0.9, 0.02 and 0.8, 0.02 and 0.7, 0.02 and 0.6, 0.02 and 0.5, 0.02 and 0.4, 0.02 and 0.3, 0.02 and 0.2, and 0.02 and 0.1.

In another aspect, the aqueous culture of the present disclosure may have an OD value of between 0.01 and 2 at 560 nm. In other aspects, the aqueous culture of the present disclosure may have an OD value at 560 nm selected from the group consisting of between 0.02 and 2, between 0.03 and 2, between 0.04 and 2, between 0.05 and 2, between 0.06 and 2, between 0.07 and 2, between 0.08 and 2, between 0.09 and 2, between 0.1 and 2, between 0.15 and 2, between 0.2 and 2, between 0.25 and 2, between 0.3 and 2, between 0.35 and 2, between 0.4 and 2, between 0.45 and 2, between 0.5 and 2, between 0.55 and 2, between 0.6 and 2, between 0.7 and 2, between 0.8 and 2, between 0.9 and 2, between 1 and 2, between 1.25 and 2, between 1.5 and 2, between 1.75 and 2, 0.02 and 1.9, 0.02 and 1.8, 0.02 and 1.7, 0.02 and 1.6, 0.02 and 1.5, 0.02 and 1.4, 0.02 and 1.3, 0.02 and 1.2, 0.02 and 1.1, 0.02 and 1, 0.02 and 0.9, 0.02 and 0.8, 0.02 and 0.7, 0.02 and 0.6, 0.02 and 0.5, 0.02 and 0.4, 0.02 and 0.3, 0.02 and 0.2, and 0.02 and 0.1.

A method of the present disclosure may be used to harvest a NVPO from an aqueous culture where the NVPO may be at any growth phase. In another aspect, the aqueous culture of the present disclosure may be in logarithmic phase growth. In another aspect, the aqueous culture of the present disclosure may be in stationary phase growth.

In an aspect, the aqueous culture of the present disclosure may have more than one species of NVPO. In one aspect, the aqueous culture of the present disclosure may have two species of NVPO. In another aspect, the aqueous culture of the present disclosure may have three species of NVPO. In yet another aspect, one or more of the more than one species of NVPO in the aqueous culture of the present disclosure may be transgenic.

In an aspect of the present disclosure, the aqueous culture of the present disclosure may have two or more species of NVPO selected from the genus Spirulina. In another aspect, the aqueous culture of the present disclosure may have two or more species of NVPO selected from the genus Scenedesmus. In a further aspect, the aqueous culture of the present disclosure may have two or more species of NVPO selected from the genus Desmodesmus. In an aspect, the aqueous culture of the present disclosure may have two or more species of NVPO selected from the genus Leptolyngbya. In an aspect, the aqueous culture of the present disclosure may have two or more species of NVPO selected from the genus Nannochloropsis. In an aspect, the aqueous culture of the present disclosure may have two or more species of NVPO selected from the genus Tetraselmis. In an aspect, the two or more species of NVPO may be transgenic.

In an aspect, the aqueous culture of the present disclosure may have two species of NVPO, one species selected from one genus and a second species selected from a second genus. In an aspect, the first genus may be Spirulina and the second genus may be Scenedesmus. In an aspect, the first genus may be Spirulina and the second genus may be Desmodesmus. In an aspect, the first genus may be Spirulina and the second genus may be Leptolyngbya. In an aspect, the first genus may be Scenedesmus and the second genus may be Leptolyngbya. In yet another aspect of the present disclosure, the first genus may be Leptolyngbya and the second genus may be Desmodesmus.

In a further aspect, the aqueous culture of the present disclosure may have three species of NVPO selected from a genus. In an aspect, the aqueous culture of the present disclosure may have three species of NVPO selected from the genus Spirulina. In another aspect, the aqueous culture of the present disclosure may have three species of NVPO selected from the genus Scenedesmus. In a further aspect, the aqueous culture of the present disclosure may have three species of NVPO selected from the genus Desmodesmus. In a further aspect, the aqueous culture of the present disclosure may have three species of NVPO selected from the genus Tetraselmis. In an aspect, one, two or three species of NVPO may be transgenic.

In an aspect, the aqueous culture of the present disclosure may have three species of NVPO, one species selected from one genus, a second species selected from a second genus and a third species selected from a third genus. In an aspect, the first genus may be Spirulina, the second genus may be Scenedesmus, and the third genus may be Leptolyngbya. In an aspect, the first genus may be Spirulina, the second genus may be Desmodesmus, and the third genus may be Leptolyngbya. In an aspect, one or more of the three species of NVPO may be transgenic. In an aspect, the aqueous culture of the present disclosure may comprise 4, 5, 6, 7, 8, 9, 10 or more combinations of species of NVPO selected from the genera of Spirulina, Scenedesmus, Desmodesmus and Leptolyngbya.

In one aspect, a method of the present disclosure is to harvest a NVPO from fresh water. In another aspect, a method of the present disclosure is to harvest a NVPO from brackish water. In another aspect, a method of the present disclosure is to harvest a NVPO from saline water. In another aspect, a method of the present disclosure is to harvest a NVPO from brine water.

The aqueous culture containing the NVPO of the present disclosure can comprise water from any natural source without treatment and/or without supplementation. The water can be fresh water, brackish water, or sea water. In some aspects, the aqueous environment may contain more than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1., 3.2, 3.3, 3.4, 3.5, 3.6, 3.7, 3.8, 3.9, 4.0, 4.1, 4.2, or 4.3 molar concentrations of sodium chloride. In other aspects, the aqueous environment may contain sodium chloride at a concentration selected from the group consisting of between 0.1 and 4.3, between 0.2 and 4.3, between 0.3 and 4.3, between 0.4 and 4.3, between 0.5 and 4.3, between 0.6 and 4.3, between 0.7 and 4.3, between 0.8 and 4.3, between 0.9 and 4.3, between 1.0 and 4.3, between 1.1 and 4.3, between 1.2 and 4.3, between 1.3 and 4.3, between 1.4 and 4.3, between 1.5 and 4.3, between 1.6 and 4.3, between 1.7 and 4.3, between 1.8 and 4.3, between 1.9 and 4.3, between 2.0 and 4.3, between 2.1 and 4.3, between 2.2 and 4.3, between 2.3 and 4.3, between 2.4 and 4.3, between 2.5 and 4.3, between 2.6 and 4.3, between 2.7 and 4.3, between 2.8 and 4.3, between 2.9 and 4.3, between 3.0 and 4.3, between 3.1, and 4.3, between 3.2 and 4.3, between 3.3 and 4.3, between 3.4 and 4.3, between 3.5 and 4.3, between 3.6 and 4.3, between 3.7 and 4.3, between 3.8 and 4.3, between 3.9 and 4.3, between 4.0 and 4.3, between 4.1 and 4.3, and between 4.2 and 4.3 molar concentrations of sodium chloride. One of skill in the art will recognize that other salts (sodium salts, calcium salts, potassium salts, etc.) may also be present in the aqueous environment.

An alternative method of measuring water quality is total dissolved solids (TDS). TDS is well known in the area of water quality and is a measure of the combined content of organic and inorganic substances dissolved in the water. As used herein, TDS may be measured in milligram/liter (mg/L) or parts per thousand (ppt), wherein one ppt equals to approximately 1000 mg/L. In general, fresh water has a TDS of less than 1500 mg/L, brackish water has a TDS of from 1500 to 35000 mg/L and saline water has a TDS of greater than 35000 mg/L. Brine water has a TDS over 100000 mg/L. Thus, in some aspects, the aqueous environment can have a TDS of up to 1500, 2000, 2500, 3000, 3500, 4000, 4500, 5000, 5500, 6000, 6500, 7000, 7500, 8000, 8500, 9000, 10000, 10500, 11000, 11500, 12000, 12500, 13000, 13500, 14000, 14500, 15000, 16000, 16500, 17000, 17500, 18000, 18500, 19000, 20000, 20500, 22000, 22500, 22000, 22500, 23000, 23500, 24000, 24500, 25000, 26000, 26500, 27000, 27500, 28000, 28500, 29000, 30000, 30500, 31000, 33500, 32000, 32500, 33000, 33500, 34000, 34500, 35000, 36000, 36500, 37000, 37500, 38000, 38500, 39000, 40000, 40500, 41000, 41500, 42000, 42500, 43000, 43500, 44000, 44500, 45000, 46000, 46500, 47000, 47500, 48000, 48500, 49000 mg/L.

In another aspect, the aqueous environment can have a TDS selected from the group consisting of between 500 and 1500, between 1500 and 35000, between 2000 and 35000, between 2500 and 35000, between 3000 and 35000, between 3500 and 35000, between 4000 and 35000, between 4500 and 35000, between 5000 and 35000, between 5500 and 35000, between 6000 and 35000, between 6500 and 35000, between 7000 and 35000, between 7500 and 35000, between 8000 and 35000, between 8500 and 35000, between 9000 and 35000, between 10000 and 35000, between 10500 and 35000, between 11000 and 35000, between 11500 and 35000, between 12000 and 35000, between 12500 and 35000, between 13000 and 35000, between 13500 and 35000, between 14000 and 35000, between 14500 and 35000, between 15000 and 35000, between 16000 and 35000, between 16500 and 35000, between 17000 and 35000, between 17500 and 35000, between 18000 and 35000, between 18500 and 35000, between 19000 and 35000, between 20000 and 35000, between 20500 and 35000, between 22000 and 35000, between 22500 and 35000, between 22000 and 35000, between 22500 and 35000, between 23000 and 35000, between 23500 and 35000, between 24000 and 35000, between 24500 and 35000, between 25000 and 35000, between 26000 and 35000, between 26500 and 35000, between 27000 and 35000, between 27500 and 35000, between 28000 and 35000, between 28500 and 35000, between 29000 and 35000, between 30000 and 35000, between 30500 and 35000, between 31000 and 35000, between 33500 and 35000, between 32000 and 35000, between 32500 and 35000, between 33000 and 35000, between 33500 and 35000, between 34000 and 35000, between 34500 and 35000, and between 35000 and 49000 mg/L.

Another way to classify water is by salinity. Salinity is a measure of the total dissolved salts in water and is traditionally measured in parts per thousand (‰). In certain aspects the aqueous environment has a salinity of less than 0.5‰, from 0.5 to 3‰, from 4 to 29‰, from 30 to 50‰, or greater than 50‰. In other aspects, the aqueous environment may be water that is not from a natural source. That is, the water composition and/or chemistry may be modified to provide the desired environment for the growth of the microorganism. For example, and without limitation, in one aspect the salt concentration of the water may be increased or decreased. In another aspect, the pH of the water may be raised or lowered. In still another aspect, the concentration of CO₂ in the water may be increased.

An aqueous culture of the present disclosure may comprise a defined or undefined medium. In one aspect, the culture may be in untreated water. In an aspect, the untreated water may be water obtainable from a natural source such as a river, lake, aquifer, ocean or a pond. In another aspect, the culture may be in brackish water having a total dissolved solids (TDS) between 1500 and 35000 mg/L. In yet another aspect, the culture may be in salt water. In an aspect, the culture may be in recycled water obtainable from a sewage or waste water treatment plant, or waste water from an industrial process such as power production and the like. In an aspect of the present disclosure, the untreated water may be aquifer water. In a further aspect, the untreated water may be aquifer water that is not suitable for agriculture. In yet another aspect, the aquifer water may be aquifer water with an elevated total dissolved solids (TDS).

Some of the photosynthetic organisms which may be used are halophilic (e.g., Dunaliella salina, Dunaliella viridis, or Dunaliella tertiolecta). For example, D. salina can grow in ocean water and salt lakes (total dissolved solids (TDS) from about 3000 to about 40000 mg/L and liquid media with high TDS (e.g., artificial seawater medium, seawater nutrient agar, brackish water medium, seawater medium, etc.). In some aspects, a photosynthetic organism for use of the present disclosure can be grown in a liquid environment which is about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, or about 4.3 molar or higher concentrations of sodium chloride. One of skill in the art will recognize that other salts (sodium salts, calcium salts, potassium salts, etc.) may also be present in the aqueous environments.

A method of the present disclosure may be used to harvest a NVPO from an aqueous environment with a wide range of pH. In one aspect, a method of the present disclosure is to harvest a NVPO from an aqueous environment with a pH value between 1 and 13. In another aspect, a method of the present disclosure is to harvest a NVPO from an aqueous environment with a pH value selected from the group consisting of between 2 and 13, between 3 and 13, between 4 and 13, between 5 and 13, between 6 and 13, between 7 and 13, between 8 and 13, between 9 and 13, between 10 and 13, between 11 and 13, between 12 and 13, between 2 and 12, between 3 and 12, between 4 and 12, between 5 and 12, between 6 and 12, between 7 and 12, between 8 and 12, between 9 and 12, between 10 and 12, between 11 and 12, between 2 and 11, between 3 and 11, between 4 and 11, between 5 and 11, between 6 and 11, between 7 and 11, between 8 and 11, between 9 and 11, between 10 and 11, between 2 and 10, between 3 and 10, between 4 and 10, between 5 and 10, between 6 and 10, between 7 and 10, between 8 and 10, between 9 and 10, between 2 and 9, between 3 and 9, between 4 and 9, between 5 and 9, between 6 and 9, between 7 and 9, between 8 and 9, between 2 and 8, between 3 and 8, between 4 and 8, between 5 and 8, between 6 and 8, between 7 and 8, between 2 and 7, between 3 and 7, between 4 and 7, between 5 and 7, between 6 and 7, between 2 and 6, between 3 and 6, between 4 and 6, between 5 and 6, between 2 and 5, between 3 and 5, between 4 and 5, between 2 and 4, between 3 and 4, and between 2 and 3. In another aspect, a method of the present disclosure is to harvest a NVPO from an aqueous environment with a pH value between 7.80 and 9.34.

A method of the present disclosure is for harvesting a non-vascular photosynthetic organism (NVPO) from an aqueous culture, and comprises mixing an effective amount of a polymer flocculant with the aqueous culture to form flocs of the NVPO, introducing dissolved air into the mixed aqueous culture, and collecting the flocs of the NVPO. Another method of the present disclosure is for harvesting a non-vascular photosynthetic organism (NVPO) from an aqueous culture without the aid of dissolved air flotation, and comprises mixing an effective amount of a polymer flocculant with the aqueous culture to form flocs of the NVPO, and collecting the flocs of the NVPO. Yet another method of the present disclosure is for harvesting a non-vascular photosynthetic organism (NVPO) from an aqueous culture without the aid of dissolved air flotation, and comprises mixing an effective amount of a polymer flocculant and/or a coagulant with the aqueous culture to cause the NVPO to sink or increase the speed of settling, and collecting the NVPO by means of a clarifier. A further method of the present disclosure is for harvesting a non-vascular photosynthetic organism (NVPO) from an aqueous culture in a pond directly, and comprises mixing an effective amount of a polymer flocculant with the aqueous culture to form flocs of the NVPO, and collecting the flocs of the NVPO.

NVPOs can typically be grown on a simple defined medium with light as the sole energy source. In most cases fluorescent light bulbs at a distance of 1-2 feet are adequate to supply energy for growth. Bubbling with air or 5% CO₂ may improve the growth rate. If the lights are turned on and off at regular intervals (either 12:12 or 14:10 hours of light:dark) the cells of some NVPOs can be synchronized.

Because photosynthetic organisms such as algae require sunlight, CO₂ and water for growth, they can be cultivated in open ponds and lakes. Due to the fact that these are open system, they are much more vulnerable to being contaminated. One challenge with using open systems is that the NVPO of interest may not necessarily be the quickest to reproduce. This creates a problem where other species colonize the liquid environment. In addition, in open systems there is relatively less control over water temperature, CO₂ concentration, and lighting conditions. These imply that the growing season is largely dependent on location and, aside from tropical areas, is limited to the warmer months. While the above are the disadvantages with “open systems,” some of the benefits of this type of system are that it typically has lower production costs.

Another approach is to use a semi-closed system, such as covering the pond or pool with a greenhouse. While this usually results in a smaller system, it addresses many of the problems associated with an open system. It allows more species to be grown, it allows the species that are being grown to stay dominant, it extends the growing season, and if the greenhouse is heated, production can continue year round. It is also possible to increase the amount of CO₂ in these semi-closed systems, thus again increasing the rate of growth of algae.

A variation of the pond system is an artificial pond e.g., a raceway pond. In raceway ponds, the algae, water and nutrients circulate around a “racetrack.” By providing water movement, for example by the use of paddlewheels, algae are kept suspended in the water, and are circulated back to the surface on a regular frequency. Raceway ponds are usually kept shallow because the algae need to be exposed to sunlight, and sunlight can only penetrate the pond water to a limited depth. However, depth can be varied according to the wavelength(s) utilized by an organism. The ponds can be operated in a continuous manner, with CO₂ and nutrients being constantly fed to the ponds, while algae-containing water is removed at the other end.

Alternatively, algae could be grown in closed structures such as photobioreactors, where the environment is under stricter control than in open ponds. While the costs of setting up and operating a photobioreactor would be higher than for those for open ponds, the efficiency and higher yields from these photobioreactors could be significantly higher as well, thus offsetting the initial cost disadvantage in the medium and long run.

A photobioreactor is a bioreactor that incorporates some type of light source. A pond covered with a greenhouse could also be considered a photobioreactor. Because these systems are closed everything that the algae need to grow, (carbon dioxide, water and light) need to be introduced into the system.

Photobioreactors can be set up to be continually harvested (the majority of the larger cultivation systems), or by harvesting a batch at a time (like polyethlyene bag cultivation). A batch photobioreactor is set up with nutrients and algal seed, and allowed to grow until the batch is harvested. A continuous photobioreactor is harvested either continually, as daily, or more frequently. Some types of photobioreactors include glass, plastic tubes, tanks, plastic sleeves or bags. Some sources that can be used to provide the light energy required to sustain photosynthesis include fluorescent bulbs LEDs, or natural sunlight.

One organism that can be cultured as described herein is a commonly used species C. reinhardtii. Cells of this species are haploid, and can grow on a simple medium of inorganic salts, using photosynthesis to provide energy. This organism can also grow in total darkness if acetate is provided as a carbon source. C. reinhardtii can be readily grown at room temperature under standard fluorescent lights. In addition, the cells can be synchronized by placing them on a light-dark cycle. Other methods of culturing C. reinhardtii cells are known to one of skill in the art. Methods for culturing organisms of the present disclosure are known in the art, for example, in Vonshak, A. Spirulina Platensis (Arthrospira): Physiology, Cell-Biology And Biotechnology. 1997. CRC Press, Andersen, A. Algal Culturing Techniques. 2005. Elsevier Academic Press, Chen et al. (2011) “Cultivation, photobioreactor design and harvesting of microalgae for biodiesel production: A critical review,” Bioresource Technology 102:71-81, Rodolfi et al., “Microalgae for oil: Strain selection, induction of lipid synthesis and outdoor mass cultivation in a low-cost photobioreactor”, Biotechnology and Bioengineering 102:100-112 (2009), and Ugwu et al., “Photobioreactors for mass cultivation of algae,” Bioresource Technology 99:4021-4028 (2008), each of which are hereby incorporated by reference in their entirety.

A method of the present disclosure is for harvesting a non-vascular photosynthetic organism (NVPO) from an aqueous culture, and comprises mixing an effective amount of a polymer flocculant with the aqueous culture to form flocs of the NVPO, introducing dissolved air into the mixed aqueous culture, and collecting the flocs of the NVPO. Another method of the present disclosure is for harvesting a non-vascular photosynthetic organism (NVPO) from an aqueous culture without the aid of dissolved air flotation, and comprises mixing an effective amount of a polymer flocculant with the aqueous culture to form flocs of the NVPO, and collecting the flocs of the NVPO. Yet another method of the present disclosure is for harvesting a non-vascular photosynthetic organism (NVPO) from an aqueous culture without the aid of dissolved air flotation, and comprises mixing an effective amount of a polymer flocculant and/or coagulant with the aqueous culture of the NVPO to cause settling or increase the settling rate of the NVPO, and collecting the NVPO using a clarifier. A further method of the present disclosure is for harvesting a non-vascular photosynthetic organism (NVPO) from an aqueous culture in a pond directly, and comprises mixing an effective amount of a polymer flocculant with the aqueous culture to form flocs of the NVPO, and collecting the flocs of the NVPO.

In one aspect, the mixing step of a method of the present disclosure comprises gentle mixing. In another aspect, the mixing step of a method of the present disclosure comprises mild mixing. In a further aspect, the mixing step of a method of the present disclosure comprises vigorous mixing. In another aspect, the mixing step of a method of the present disclosure is performed with a paddle wheel.

As used herein, a “flocculant” relates to any compound, agent, or substance that promotes flocculation. A “polymer flocculant” relates to a polymer that promotes flocculation.

As used herein, “flocculation” relates to a process of contact and adhesion whereby individual particles of a dispersion form clusters of two or more particles. In general, the first stage of flocculation is the aggregation of suspended solids into larger particles resulting from the interaction of the flocculant with the surface charge of the suspended solids. The second stage involves the coalescing of aggregates into large flocs that settle out of suspension.

Polymers may be used as flocculants. As used herein, a “polymer” relates to a large molecule (macromolecule) composed of repeating structural units of monomers wherein monomers are connected by covalent bonds. A polymer may be natural or synthetic.

Monomers, and resulting polymers may be non-ionic, cationic, or anionic. A polymer with a charged group in its monomer subunit is also called a polyelectrolyte. A polymer of the present disclosure may be a copolymer which is made up of two or more monomers. A polymer of the present disclosure may be in a powder (dry), liquid, or latex (emulsion) form. Dry polymers can be up to 100% active and are in a dry powder form. Polymers in an emulsion form are usually 60% active polymer and are stored in an oil base.

Examples of non-ionic monomers include, but are not limited to, acrylamide, methacrylamide, N-vinylmethylacetamide, N-vinyl methyl formamide, vinyl acetate, vinyl pyrrolidone, methyl methacrylate, methacrylic esters, other acrylic or ethylenically unsaturated esters, styrene, and acrylonitrile. Further exemplary non-ionic monomers include dialkylaminoalkymethacrylamide and sulphomethylated acrylamide.

Examples of cationic monomers are dialkylaminoalkylacrylates and methacrylates, especially dialkylamino ethyl acrylate, and their quaternary or acid salts, and dialkylaminoalkylacrylamides or methacrylamides and their quaternary or acid salts and Mannich products, such as quaternized dialkylaminoethylacrylamides. Alkyl groups are generally C₁₋₄ hydrocarbons that may be either branched straight chain. Quaternary salts include quaternary ammonium salts, such as methylated quaternary ammonium salts. For example, cationic monomers include dimethyl aminoethyl acrylate methyl chloride.

Examples of anionic monomers includes, e.g., acrylic acid, sodium acrylate, sodium methacrylate, ammonium acrylate, ammonium methacrylate, methacrylic acid, itaconic acid, 2-acrylamide 2-methyl propane sulphonate, sulphopropylacrylate, methacrylate, or other forms of these carboxylic or sulphonic acids.

Polymers can be used in a flocculation process. In addition to reducing or neutralizing the surface charge on cells, polymer flocculants can bring particles together by physically linking one or more particles through a process called bridging.

Flocculation effectiveness of a polymer depends on many factors, including the molecular weight of the polymer, the charge density on the polymer molecule, the dose used, the concentration of the NVPO to be harvested, the ionic strength and pH of the aqueous culture, and the extent of mixing in the system. Generally, high molecular weight polymers are better bridging agents. A high charge density tends to unfold a polymer molecule, improving its bridging performance and the ability to neutralize the surface change on cells. A high concentration of NVPO in an aqueous culture of NVPO helps flocculation, because the cell-cell encounters are more frequent in concentrated suspensions. A certain low level of mixing is useful as it helps bring cells together; however, excessive shear forces can disrupt flocs.

Cationic polymers doses of between 1 and 10 mg/L can induce flocculation of freshwater algae; however, a high salinity of the marine environment can inhibit flocculation by polymers. At high ionic strengths, cationic polymers tend to fold tightly and fail to bridge microalgal cells. In one aspect, the cationic polymer used in the present disclosure is a polymer of diallyldimethylammonium chloride (polyDADMAC). In another aspect, the cationic polymer used in the present disclosure is a copolymer of acrylamide with cationic acrylic acid derivative. In one aspect, the cationic polymer used in the present disclosure is not an inorganic polymer. In another aspect, the cationic polymer used in the present disclosure is not a natural polymer. In a further aspect, the cationic polymer used in the present disclosure is not chitin or any of its derivatives such as chitosans.

Polymers in the present disclosure include, without limitation, Nalco polymers 7190 PLUS, 7193 PLUS, 9913, 7191 PLUS, 8110 PULV, 9905, 71305, 7199, 1T03, 9914, 9907, 7192 PLUS, GR-206, 9908, 71303, 71302, 1T69, 7751, 7139 PLUS, GR-503, 9909, 71301, GR-201, 71300, 1404, GR-204, 71306, 71307, 9916, GR-505, 7181, 8181, 7182, 8182, 7769, 7766 PLUS, 7763, 8184, 71325, GR-105, GR-602, 7744, 7768, 1C34, 7767, 7878, 71302, 1T69, 7751, 7139 PLUS, GR-503, 9909, 71301, GR-201, 71300, 1404, GR-204, 71306, 71307, 9916, GR-505, 7181, 8181, 7182, 8182, 7769, 7766 PLUS, 7763, 8184, 71325, GR-105, GR-602, 7744, 7768, 1C34, 7767, and 7878.

In one aspect, a method of the present disclosure comprises the use of a natural polymer. In another aspect, a method of the present disclosure comprises the use of a synthetic polymer. In one aspect, a method of the present disclosure comprises the use of an anionic polymer. In another aspect, a method of the present disclosure comprises the use of a cationic polymer. In another aspect, a method of the present disclosure comprises the use of a non-ionic polymer. In one aspect, a method of the present disclosure comprises the use of a dry polymer. In another aspect, a method of the present disclosure comprises the use of an emulsion polymer. In one aspect, a method of the present disclosure comprises the use of a combination of an anionic polymer and a cationic polymer. In another aspect, a method of the present disclosure comprises the use of a combination of an anionic and a cationic coagulant.

In one aspect, a method of the present disclosure comprises the use of a copolymer of acrylamide with cationic acrylic acid derivative. In another aspect, a method of the present disclosure comprises the use of polymer PRAESTOL® 859 BS from Ashland.

In another aspect, a method of the present disclosure does not comprise the use of one or more polymers selected from the group consisting of Nalco polymers 7190 PLUS, 7193 PLUS, 9913, 7191 PLUS, 8110 PULV, 9905, 71305, 7199, 1T03, 9914, 9907, 7192 PLUS, GR-206, 9908, 71303, 71302, 1T69, 7751, 7139 PLUS, GR-503, 9909, 71301, GR-201, 71300, 1404, GR-204, 71306, 71307, 9916, GR-505, 7181, 8181, 7182, 8182, 7769, 7766 PLUS, 7763, 8184, 71325, GR-105, GR-602, 7744, 7768, 1C34, 7767, 7878, 71302, 1T69, 7751, 7139 PLUS, GR-503, 9909, 71301, GR-201, 71300, 1404, GR-204, 71306, 71307, 9916, GR-505, 7181, 8181, 7182, 8182, 7769, 7766 PLUS, 7763, 8184, 71325, GR-105, GR-602, 7744, 7768, 1C34, 7767, and 7878.

In one aspect, a method of the present disclosure comprises the use of a polymer flocculant having a molecular weight of at least 0.5 MDa. As used herein, “molecular weight of a polymer” relates to the average molecular weight of a polymer. It may refer to the Number Average Molecular Weight (Mn), calculated as the total weight of all the polymer molecules in a sample, divided by the total number of polymer molecules in a sample. It may also refer to the Weight Average Molecular Weight (Mw) which is determined by methods that are sensitive to the molecular size rather than just their number, such as light scattering techniques. Commonly used average molecular weight of a polymer can be determined by gel permeation chromatography (GPC) and size exclusion chromatography (SEC).

In another aspect, a method of the present disclosure comprises the use of a polymer flocculant having a molecular weight selected from the group consisting of at least 0.5, at least 1.0, at least 1.5, at least 2.0, at least 2.5, at least 3.0, at least 3.5, at least 4.0, at least 4.5, at least 5.0, at least 5.5, at least 6.0, at least 6.5, at least 7.0, at least 7.5, at least 8.0, at least 8.5, at least 9.0, at least 9.5, and at least 10.0 MDa. In another aspect, a method of the present disclosure comprises the use of a polymer flocculant having a molecular weight selected from the group consisting of at least 2.0, at least 2.5, at least 3.0, at least 3.5, at least 4.0, at least 4.5, at least 5.0, at least 5.5, at least 6.0, at least 6.5, at least 7.0, at least 7.5, at least 8.0, at least 8.5, at least 9.0, at least 9.5, and at least 10.0 MDa. In another aspect, a method of the present disclosure comprises the use of a polymer flocculant having a molecular weight selected from the group consisting of at least 4.0, at least 4.5, at least 5.0, at least 5.5, at least 6.0, at least 6.5, at least 7.0, at least 7.5, at least 8.0, at least 8.5, at least 9.0, at least 9.5, and at least 10.0 MDa.

In another aspect, a method of the present disclosure comprises the use of a polymer flocculant having a molecular weight selected from the group consisting of between 0.5 and 40.0, between 1.0 and 40.0, between 1.5 and 40.0, between 2.0 and 40.0, between 2.5 and 40.0, between 3.0 and 40.0, between 3.5 and 40.0, between 4.0 and 40.0, between 4.5 and 40.0, between 5.0 and 40.0, between 5.5 and 40.0, between 6.0 and 40.0, between 6.5 and 40.0, between 7.0 and 40.0, between 7.5 and 40.0, between 8.0 and 40.0, between 8.5 and 40.0, between 9.0 and 40.0, between 9.5 and 40.0, between 10.0 and 40.0, between 11.0 and 40.0, between 12.0 and 40.0, between 13.0 and 40.0, between 14.0 and 40.0, between 15.0 and 40.0, between 16.0 and 40.0, between 17.0 and 40.0, between 18.0 and 40.0, between 19.0 and 40.0, between 20.0 and 40.0, between 25.0 and 40.0, between 30.0 and 40.0, between 35.0 and 40.0 MDa.

In another aspect, a method of the present disclosure comprises the use of a polymer flocculant having a molecular weight selected from the group consisting of between 5.0 and 40.0, between 5.5 and 40.0, between 6.0 and 40.0, between 6.5 and 40.0, between 7.0 and 40.0, between 7.5 and 40.0, between 8.0 and 40.0, between 8.5 and 40.0, between 9.0 and 40.0, between 9.5 and 40.0, between 10.0 and 40.0, between 11.0 and 40.0, between 12.0 and 40.0, between 13.0 and 40.0, between 14.0 and 40.0, between 15.0 and 40.0, between 16.0 and 40.0, between 17.0 and 40.0, between 18.0 and 40.0, between 19.0 and 40.0, between 20.0 and 40.0, between 25.0 and 40.0, between 30.0 and 40.0, between 35.0 and 40.0 MDa.

In another aspect, a method of the present disclosure comprises the use of a polymer flocculant having a molecular weight selected from the group consisting of between 10.0 and 40.0, between 11.0 and 40.0, between 12.0 and 40.0, between 13.0 and 40.0, between 14.0 and 40.0, between 15.0 and 40.0, between 16.0 and 40.0, between 17.0 and 40.0, between 18.0 and 40.0, between 19.0 and 40.0, between 20.0 and 40.0, between 25.0 and 40.0, between 30.0 and 40.0, between 35.0 and 40.0 MDa.

In another aspect, a method of the present disclosure comprises the use of a polymer flocculant having a molecular weight selected from the group consisting of between 15.0 and 40.0, between 16.0 and 40.0, between 17.0 and 40.0, between 18.0 and 40.0, between 19.0 and 40.0, between 20.0 and 40.0, between 25.0 and 40.0, between 30.0 and 40.0, between 35.0 and 40.0 MDa.

In another aspect, a method of the present disclosure comprises the use of a polymer flocculant having a molecular weight selected from the group consisting of between 20.0 and 40.0, between 25.0 and 40.0, between 30.0 and 40.0, between 35.0 and 40.0 MDa.

In one aspect, the polymer used by a method of the present disclosure has a charge density (mol %) between 1% and 100%. As used herein, “charge density” of a polymer relates to a measure of the electrical charge per length or volume of polymer. In another aspect, the polymer of the present disclosure has a charge density (mol %) selected from the group consisting of between 5% and 100%, between 5% and 90%, between 5% and 80%, between 5% and 70%, between 5% and 60%, between 5% and 50%, between 5% and 40%, between 5% and 30%, between 5% and 20%, between 5% and 10%, between 10% and 100%, between 10% and 90%, between 10% and 80%, between 10% and 70%, between 10% and 60%, between 10% and 50%, between 10% and 40%, between 10% and 30%, between 10% and 20%, between 20% and 100%, between 20% and 90%, between 20% and 80%, between 20% and 70%, between 20% and 60%, between 20% and 50%, between 20% and 40%, between 20% and 30%, between 30% and 100%, between 30% and 90%, between 30% and 80%, between 30% and 70%, between 30% and 60%, between 30% and 50%, between 30% and 40%, between 40% and 100%, between 40% and 90%, between 40% and 80%, between 40% and 70%, between 40% and 60%, between 40% and 50%, between 50% and 100%, between 50% and 90%, between 50% and 80%, between 50% and 70%, between 50% and 60%, between 60% and 100%, between 60% and 90%, between 60% and 80%, between 60% and 70%, between 70% and 100%, between 70% and 90%, between 70% and 80%, between 80% and 100%, between 80% and 90%, and between 90% and 100%.

In one aspect, a method of the present disclosure comprises a polymer dosing of less than 100 ppm. As used herein, “polymer dosing” or “polymer dosage” relates to the working concentration of a polymer (weight/volume) present in a to-be-flocculated NVPO-containing aqueous culture. Polymer dosing used in the present disclosure may be greater than 0, but less than an amount selected from the group consisting of 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 10, 10.5, 11, 11.5, 12, 12.5, 13, 13.5, 14, 14.5, 15, 16, 16.5, 17, 17.5, 18, 18.5, 19, 20, 20.5, 22, 22.5, 22, 22.5, 23, 23.5, 24, 24.5, 25, 26, 26.5, 27, 27.5, 28, 28.5, 29, 30, 30.5, 33, 33.5, 32, 32.5, 33, 33.5, 34, 34.5, 35, 36, 36.5, 37, 37.5, 38, 38.5, 39, 40, 40.5, 44, 44.5, 42, 42.5, 43, 43.5, 44, 44.5, 45, 46, 46.5, 47, 47.5, 48, 48.5, 49, 50, 60, 70, 80, 90, and 100 parts per million (ppm). As used herein, one “part per million (ppm)” is equivalent to one milligram per liter (mg/L).

In another aspect, a method of the present disclosure comprises a polymer dosing selected from the group consisting of greater than 0, but less than 90 ppm, less than 80 ppm, less than 70 ppm, less than 60 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, less than 1 ppm, and less than 0.5 ppm. In another aspect, a method of the present disclosure comprises a polymer dosing selected from the group consisting of less than 70 ppm, less than 60 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, less than 1 ppm, and less than 0.5 ppm.

In another aspect, a method of the present disclosure comprises a polymer dosing selected from the group consisting of greater than 0, but less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, less than 1 ppm, and less than 0.5 ppm. In another aspect, a method of the present disclosure comprises a polymer dosing selected from the group consisting of greater than 0, but less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, less than 1 ppm, and less than 0.5 ppm. In another aspect, a method of the present disclosure comprises a polymer dosing selected from the group consisting of greater than 0, but less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, less than 1 ppm, and less than 0.5 ppm. In another aspect, a method of the present disclosure comprises a polymer dosing selected from the group consisting of greater than 0, but less than 10 ppm, less than 9 ppm, less than 8 ppm, less than 7 ppm, less than 6 ppm, less than 5 ppm, less than 4 ppm, less than 3 ppm, less than 2 ppm, less than 1 ppm, and less than 0.5 ppm.

In another aspect, a method of the present disclosure comprises a polymer dosing selected from the group consisting of greater than 0, but less than 8 ppm, less than 7 ppm, less than 6 ppm, less than 5 ppm, less than 4 ppm, less than 3 ppm, less than 2 ppm, less than 1 ppm, and less than 0.5 ppm. In another aspect, a method of the present disclosure comprises a polymer dosing selected from the group consisting of greater than 0, but less than 6 ppm, less than 5 ppm, less than 4 ppm, less than 3 ppm, less than 2 ppm, less than 1 ppm, and less than 0.5 ppm. In another aspect, a method of the present disclosure comprises a polymer dosing selected from the group consisting of greater than 0, but less than 6 ppm, less than 5 ppm, less than 4 ppm, less than 3 ppm, less than 2 ppm, less than 1 ppm, and less than 0.5 ppm. In another aspect, a method of the present disclosure comprises a polymer dosing selected from the group consisting of greater than 0, but less than 4 ppm, less than 3 ppm, less than 2 ppm, less than 1 ppm, and less than 0.5 ppm. In another aspect, a method of the present disclosure comprises a polymer dosing selected from the group consisting of greater than 0, but less than 2 ppm, less than 1 ppm, and less than 0.5 ppm.

A method of the present disclosure may use polymers which have no significant effects on a to-be-harvested NVPO or the aqueous environment of a to-be-harvested aqueous culture. In one aspect, a method of the present disclosure uses a polymer at a dosing which has no significant impacts on the growth of a to-be-harvested NVPO as compared to the same NVPO that has not been treated with the polymer. In another aspect, a method of the present disclosure uses a polymer at a dosing which has no significant impacts on pH of the aqueous culture. In a further aspect, a method of the present disclosure uses a polymer at a dosing which has no significant impacts on the level of nitrate or phosphate in the aqueous culture.

In one aspect, a polymer stock concentration between 0.1-1% (weight/volume) is used in a method of the present disclosure. As used herein, a “polymer stock concentration” relates to the concentration of a polymer stock solution. Polymer stock concentrations typically range between 0.1-1% (weight/volume). Stock solutions of higher concentrations may have an excess viscosity and hard to work with.

As used herein, “viscosity” of a polymer solution relates to a measure of the resistance of a solution which is being deformed by either shear stress or tensile stress. Viscosity in the present disclosure may refer to thickness or internal friction of a solution. Viscosity of a polymer solution depends on the size, shape, and molecular weight of the polymer.

In another aspect, a method of the present disclosure uses a polymer stock concentration selected from the group consisting of 0.1%, 0.25%, 0.5%, 0.75%, and 1%.

Proper mixing is required when making a polymer stock solution to ensure polymers are properly wetted. For dry polymers, mixing may be achieved using an aspirator-type disperser that draws solid particles into a water stream using a vacuum created by water pressure. A water pressure of 30 psi or greater may be necessary for proper mixing. Wetted polymers from the aspirator are then discharged into a vessel equipped with a high torque mixer capable of stirring the entire tank at 250-400 rpm. Larger impellers may be used to increase the horsepower of the mixer, if necessary. Mixing speed beyond 400 rpm is not desired as shearing of the polymer may occur. Inadequate mixing may lead to aggregation of the polymer into clumps so that the polymer never fully dissolves.

For emulsion polymers, manual mixing may be achieved by adding neat polymers into the vortex of a stirred tank at speeds between 250-400 rpm. Alternatively, commercially available automatic feed systems that provide in-line mechanical mixing may be used. These units feature initial high energy mixing (>1000 rpm) for a short time (<15 sec) to achieve good dispersion of the product into water. This is followed by lower energy mixing (<400 rpm) for a longer period of time (10-20 min) and aging for an additional 10-20 minutes to achieve complete polymer dissolution. Specifically, two (2) milliliters of emulsion polymer are injected into the vortex of 400 milliliters of water being stirred with a mechanical mixer to prepare a 0.50 percent solution. The mixing is continued at 250-400 rpm for 10-20 minutes. For best results, the mixed solution is allowed to age for an additional 10-20 minutes before testing. For both dry and emulsion polymers, stocks are prepared at a concentration between 0.25% and 1%.

In one aspect, a method of the present disclosure comprises mixing an aqueous culture with an effective amount of a coagulant instead of a polymer flocculant.

As used herein, a “coagulant” or a “colloidal destabilizer” relates to any compound, agent, or substance that promotes coagulation. Multivalent metal salts ferric chloride (FeCl₃), aluminum sulfate (Al₂(SO₄)₃, alum) and ferric sulfate (Fe₂(SO₄)₃) are effective coagulants. Coagulation efficiency of metal ions increases with increasing ionic charge. Coagulant may also be a blend of an organic substance and a multivalent metal salt.

As used herein, “coagulation” relates to a process by which colloidal particles and very fine solid suspensions initially present in an aqueous environment are combined into larger agglomerates that can be further separated via sedimentation, flocculation, filtration, centrifugation or other separation methods. Coagulation is often achieved by adding one or more chemicals to an aqueous solution to promote destabilization of the colloid dispersion and agglomeration of the resulting individual colloidal particles.

Coagulants in the present disclosure include, without limitation, Nalco organic or blend coagulants 71306, 71307, 9916, GR-505, 7181, 8181, 7182, 8182, 7769, 7766 PLUS, 7763, 8184, 71325, GR-105, GR-602, 7744, 7768, 1C34, 7767, 7878, LS, 8108 Plus, 8102 Plus, 8102, 8103 Plus, 71259, 8799 LS, 8799 Plus, 7135, 8105, 8190, GR-410, GR-411, GR-400, and GR-401.

In one aspect, a method of the present disclosure comprises mixing an effective amount of an organic flocculant, an inorganic flocculant, an organic coagulant, an inorganic coagulant, or combinations thereof with the aqueous culture. If more than one flocculant or more than more coagulant, or both a flocculant and a coagulant are used, their mixing may be in any order. In other aspects, the mixing of an inorganic flocculant or coagulant (organic or inorganic) may be performed before, after, or while mixing the organic polymer flocculant.

A method of the present disclosure is for harvesting a non-vascular photosynthetic organism (NVPO) from an aqueous culture, and comprises mixing an effective amount of a polymer flocculant with the aqueous culture to form flocs of the NVPO, introducing dissolved air into the mixed aqueous culture, and collecting the flocs of the NVPO. Another method of the present disclosure is for harvesting a non-vascular photosynthetic organism (NVPO) from an aqueous culture without the aid of dissolved air flotation, and comprises mixing an effective amount of a polymer flocculant with the aqueous culture to form flocs of the NVPO, and collecting the floes of the NVPO. Another method of the present disclosure is for harvesting a non-vascular photosynthetic organism (NVPO) from an aqueous culture without the aid of dissolved air flotation, and comprises mixing an effective amount of a polymer flocculant and/or coagulant with the aqueous culture cause settling or increase the rate of settling of the NVPO, and collecting the NVPO by sedimentation. A further method of the present disclosure is for harvesting a non-vascular photosynthetic organism (NVPO) from an aqueous culture in a pond directly, and comprises mixing an effective amount of a polymer flocculant with the aqueous culture to form flocs of the NVPO, and collecting the flocs of the NVPO.

Upon mixing of a polymer flocculant with a NVPO-containing aqueous culture, flocs of the NVPO form at various rates. Such rates may be affected by the type and concentration of NVPO, the type and concentration of the polymer flocculant, and conditions of the aqueous culture, e.g., salinity, TDS level, pH. Depending on the rate of floc formation, to achieve an optimal flocculation efficiency, an aqueous culture mixed with a polymer flocculant may be kept at a mixing station for an appropriate time period to allow flocs to form before proceeding to a next step. This appropriate time period is called residence time.

In one aspect, a method of the present disclosure comprises a residence time of greater than 0.25 minutes, but less than 60 minutes. In another aspect, a method of the present disclosure comprises a residence time selected from the group consisting of greater than 0.25 minutes, but less than 50 minutes, less than 40 minutes, less than 30 minutes, less than 20 minutes, less than 10 minutes, less than 9 minutes, less than 8 minutes, less than 7 minutes, less than 6 minutes, less than 5 minutes, less than 4 minutes, less than 3 minutes, less than 2 minutes, and less than 1 minute. In another aspect, a method of the present disclosure comprises a residence time selected from the group consisting of greater than 0.25 minutes, but less than 30 minutes, less than 20 minutes, less than 10 minutes, less than 9 minutes, less than 8 minutes, less than 7 minutes, less than 6 minutes, less than 5 minutes, less than 4 minutes, less than 3 minutes, less than 2 minutes, and less than 1 minute.

In another aspect, a method of the present disclosure comprises a residence time selected from the group consisting of greater than 0.25 minutes, but less than 10 minutes, less than 9 minutes, less than 8 minutes, less than 7 minutes, less than 6 minutes, less than 5 minutes, less than 4 minutes, less than 3 minutes, less than 2 minutes, and less than 1 minute. In another aspect, a method of the present disclosure comprises a residence time selected from the group consisting of greater than 0.25 minutes, but less than 8 minutes, less than 7 minutes, less than 6 minutes, less than 5 minutes, less than 4 minutes, less than 3 minutes, less than 2 minutes, and less than 1 minute.

In one aspect, a method of the present disclosure may introduce dissolved air after the mixing of a polymer flocculant with an aqueous culture. In another aspect, a method of the present disclosure may introduce dissolved air before the mixing of a polymer flocculant with an aqueous culture. In one aspect, a method of the present disclosure may introduce dissolved air while mixing a polymer flocculant with an aqueous culture.

In one aspect, a method of the present disclosure may introduce dissolved air after the mixing of a polymer flocculant with an aqueous culture. In another aspect, a method of the present disclosure may introduce dissolved air before the mixing of a polymer flocculant with an aqueous culture. In one aspect, a method of the present disclosure may introduce dissolved air while mixing a polymer flocculant with an aqueous culture.

In one aspect, a method of the present disclosure involves the use of air bubbles with an average diameter between 5 and 150 microns. In another aspect, a method of the present disclosure involves the use of air bulbs with an average diameter selected from the group consisting of between 6 and 150, between 7 and 140, between 8 and 130, between 9 and 120, between 10 and 110, between 12 and 100, between 14 and 90, between 16 and 80, between 18 and 70, between 20 and 60, between 25 and 50, and between 30 and 40 microns.

A method of the present disclosure is for harvesting a non-vascular photosynthetic organism (NVPO) from an aqueous culture, and comprises mixing an effective amount of a polymer flocculant with the aqueous culture to form flocs of the NVPO, introducing dissolved air into the mixed aqueous culture, and collecting the flocs of the NVPO. Another method of the present disclosure is for harvesting a non-vascular photosynthetic organism (NVPO) from an aqueous culture without the aid of dissolved air flotation, and comprises mixing an effective amount of a polymer flocculant with the aqueous culture to form flocs of the NVPO, and collecting the flocs of the NVPO. A further method of the present disclosure is for harvesting a non-vascular photosynthetic organism (NVPO) from an aqueous culture in a pond directly, and comprises mixing an effective amount of a polymer flocculant with the aqueous culture to form flocs of the NVPO, and collecting the flocs of the NVPO. The aforementioned methods may be used to harvest an NVPO from an aqueous culture comprising a total dissolved solids (TDS) of any level. In one aspect, the aforementioned methods may be used to harvest an NVPO from brackish water having a TDS selected from the group consisting of between 1500 and 35000, between 5000 and 35000, and between 15000 and 35000 mg/L.

The step of collecting NVPO flocs in a method of the present disclosure may be achieved by any means. In one aspect, a method of the present disclosure achieves the collecting step by skimming. In another aspect, a method of the present disclosure achieves the collecting step by sedimentation. In another aspect, a method of the present disclosure achieves the collecting step by flotation.

In one aspect, the aforementioned methods may be performed in a jar-test setting. A jar test is a pilot-scale laboratory test simulating a fullscale harvesting process and to determine the optimum concentration of coagulant or flocculant needed. A jar test is typically performed in a series of glass or plastic jars under identical conditions. The jars are injected with flocculant and or coagulant and mixed to match field conditions as closely as possible.

In another aspect, the aforementioned methods may be performed in a tank of any size or volume. In one aspect, a method of the present disclosure can be used to harvest a NVPO from a tank of at least 3 cubic meters, at least 4 cubic meters, at least 5 cubic meters, at least 6 cubic meters, at least 7 cubic meters, at least 8 cubic meters, at least 9 cubic meters, at least 10 cubic meters, at least 11 cubic meters, at least 12 cubic meters, at least 13 cubic meters, at least 14 cubic meters, at least 15 cubic meters, at least 16 cubic meters, at least 17 cubic meters, at least 18 cubic meters, at least 19 cubic meters, at least 20 cubic meters, at least 30 cubic meters, at least 40 cubic meters, at least 50 cubic meters, at least 60 cubic meters, at least 70 cubic meters, at least 80 cubic meters, at least 90 cubic meters, at least 100 cubic meters, at least 110 cubic meters, at least 120 cubic meters, at least 200 cubic meters, at least 300 cubic meters, at least 400 cubic meters, at least 500 cubic meters, at least 800 cubic meters, at least 1000 cubic meters, at least 1500 cubic meters, at least 2000 cubic meters, at least 2500 cubic meters, at least 3000 cubic meters, at least 3500 cubic meters, or at least 4000 cubic meters.

In another aspect, a method of the present disclosure can be used to harvest a NVPO from a tank having a size selected from the group consisting of between 100 and 400, between 150 and 400, between 200 and 400, between 300 and 400, between 350 and 400, between 350 and 400, between 300 and 4000, between 350 and 4000, between 400 and 4000, between 450 and 4000, between 500 and 4000, between 600 and 4000, between 700 and 4000, between 800 and 4000, between 900 and 4000, between 1000 and 4000, between 1500 and 4000, between 2000 and 4000, between 2500 and 4000, between 3000 and 4000, and between 3500 and 4000 cubic meters.

In another aspect, the aforementioned methods may be performed in a tank designed for a Dissolved Air Flotation (DAF) process. Dissolved Air Flotation (DAF) is the process of removing suspended solids, e.g., NVPOs, from an aqueous culture via the use of air bubble flotation. In a typical DAF process, an NVPO-containing aqueous culture is fed into the DAF system as an input stream. The input stream is then mixed with an aqueous solution containing dissolved air. The dissolved air in the mixed stream is released in the form of air bubbles when the mixed stream reaches a DAF tank with atmospheric pressure. Air bubbles attach to the flocs of NVPO biomass and float the flocs to the aqueous surface. The NVPO biomass float is then mechanically skimmed and removed from the DAF tank. The remaining aqueous medium in a DAF tank, also called subnatant, is returned to a NVPO culture system to support further growth of NVPO. Subnatant may also be fed into another tank with an air compressor to generate subnatant with dissolved air. Subnatant with dissolved air may be mixed with the input stream and thus introduce dissolved air in the DAF tank.

As used herein, a “floc” relates to a cluster of particles tethered together and generated by the process of flocculation. Flocs may float to the surface of an aqueous solution or settle to the bottom of an aqueous solution at a rate depending in part on the property of flocculated particles and the size of flocs. Flocs formed by the methods of the present disclosure can have an average diameter of at least about 0.05, 0.1, 0.2, 0.3, 0.5, 1, 2, 5, 10, 20, 30, 40, 50, and 60 millimeters. In some aspects, flocs formed by the methods of the present disclosure can be composed of at least 10², 10¹, 10⁴, 10⁵ individual NVPOs.

A method of the present disclosure can ensure an efficiency use of the polymer flocculant added to the aqueous culture. In one aspect, a method of the present disclosure produces harvested flocs comprising more than 50% of the polymer flocculant added into the aqueous solution. In another aspect, a method of the present disclosure produces harvested flocs comprising a percentage of the polymer flocculant added into the aqueous solution, wherein the percentage is selected from the group consisting of more than 60%, more than 70%, more than 80%, more than 90%, more than 91%, more than 92%, more than 93%, more than 94%, more than 95%, more than 96%, more than 97%, more than 98%, more than 99%, more than 99.5%, and more than 99.9% of polymer flocculant added to the aqueous solution.

In one aspect, a method of the present disclosure can achieve a harvesting efficiency of at least 50%.

As used herein, “flocculation efficiency” relates to a measurement of the percentage of NVPOs from the initial aqueous culture being harvested through the disclosed method of harvesting. The present disclosure includes two methods, one indirect and one direct, for evaluating flocculation efficiency of a polymer and the optimal dosing of a polymer. An indirect method is carried out by sampling the influent stream (pond or initial culture) of a NVPO prior to any addition of polymer and measuring the Ash Free Dry Weight (AFDW). Once polymer is added and allowed to flocculate, subnatant is sampled and the AFDW is measured. Subnatant is recycled/processed before returning to ponds and represents the NVPO that has not been bound to polymer. When calculating an indirect efficiency, the ratio of influent to subnatant is taken to determine the amount of NVPO that is extracted from the stream. The following formula is used to calculate an indirect efficiency: 1−(Subnatant AFDW/Influent AFDW)=% Efficiency.

A second method is applied when the volume of input NVPO culture is measured. The influent stream of NVPO is sampled prior to any addition of flocculant and the AFDW is measured. The volume of product harvested off the DAF (dewatered NVPO at ˜80% moisture) is compared to the total culture volume processed through the system. In this method, a graduated cylinder or appropriate sized graduate container for accurate measurement of harvested product is used. When a predetermined volume of pond has been harvested the product recovered from the DAF is sampled and an AFDW is recorded for the sample. Samples are well mixed before sampling to prevent an inhomogeneous sampling. The following formula is used to calculate a direct efficiency: (Volume product*AFDW Product)/(Volume processed*AFDW influent)=% Efficiency.

In another aspect, a method of harvesting in the present disclosure can achieve a flocculation efficiency selected from the group consisting of at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, and at least 99.9%.

A method of the present disclosure is for harvesting a non-vascular photosynthetic organism (NVPO) from an aqueous culture, and comprises mixing an effective amount of a polymer flocculant with the aqueous culture to form flocs of the NVPO, introducing dissolved air into the mixed aqueous culture, and collecting the flocs of the NVPO. Another method of the present disclosure is for harvesting a non-vascular photosynthetic organism (NVPO) from an aqueous culture without the aid of dissolved air flotation, and comprises mixing an effective amount of a polymer flocculant with the aqueous culture to form flocs of the NVPO, and collecting the flocs of the NVPO. A further method of the present disclosure is for harvesting a non-vascular photosynthetic organism (NVPO) from an aqueous culture in a pond directly, and comprises mixing an effective amount of a polymer flocculant with the aqueous culture to form flocs of the NVPO, and collecting the floes of the NVPO. Yet another method of the present disclosure is for harvesting a non-vascular photosynthetic organism (NVPO) from an aqueous culture without the aid of dissolved air flotation, and comprises mixing an effective amount of a polymer flocculant and/or a coagulant with the aqueous culture of the NVPO to cause settling or increase the rate of settling of the NVPO, and collecting the NVPO by sedimentation. The aforementioned methods may be used to harvest an NVPO from an aqueous culture comprising a total dissolved solids (TDS) of any level. In one aspect, the aforementioned methods may be used to harvest an NVPO from brackish water having a TDS selected from the group consisting of between 1500 and 35000, between 5000 and 35000, and between 15000 and 35000 mg/L.

A method of the present disclosure can generate a desirable harvest volume. As used herein, “harvest volume” relates to the volume of a biomass slurry collected by the methods of harvesting disclosed in the present disclosure.

In one aspect, a method of the present disclosure can achieve a harvest volume of less than 30% of the volume of the aqueous solution. In another aspect, a method of the present disclosure can achieve a harvest volume selected from the group consisting of less than 25%, less than 20%, less than 15%, less than 10%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, and less than 0.1% of the volume of the aqueous solution. In another aspect, a method of the present disclosure can achieve a harvest volume selected from the group consisting of less than 20%, less than 15%, less than 10%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, and less than 0.1% of the volume of the aqueous solution. In another aspect, a method of the present disclosure can achieve a harvest volume selected from the group consisting of less than 15%, less than 10%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, and less than 0.1% of the volume of the aqueous solution.

In another aspect, a method of the present disclosure can achieve a harvest volume selected from the group consisting of less than 10%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, and less than 0.1% of the volume of the aqueous solution. In another aspect, a method of the present disclosure can achieve a harvest volume selected from the group consisting of less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, and less than 0.1% of the volume of the aqueous solution. In another aspect, a method of the present disclosure can achieve a harvest volume selected from the group consisting of less than 3%, less than 2%, less than 1%, less than 0.9%, less than 0.8%, less than 0.7%, less than 0.6%, less than 0.5%, less than 0.4%, less than 0.3%, less than 0.2%, and less than 0.1% of the volume of the aqueous solution.

In another aspect, a method of the present disclosure further comprises a decanting centrifugation step to further remove water from the harvested flocs. In another aspect, a method of the present disclosure further comprises dehydrating the harvested flocs by spray drying, paddle drying, drum drying, freeze-drying, and sun drying. In another aspect, a method of the present disclosure further comprises removing the polymer flocculant from the harvested flocs. In another aspect, a method of the present disclosure further comprises feeding the harvested flocs to a solvent extraction process.

The aforementioned methods of harvesting may be performed without the aid of dissolved air flotation. In one aspect, the present disclosure includes a non-air-assisted method of harvesting a non-vascular photosynthetic organism (NVPO) from an aqueous culture by mixing an effective amount of a polymer flocculant with the aqueous culture to form flocs of the NVPO, and collecting the flocs of the NVPO, where the method does not involve dissolved air. In another aspect, the present disclosure includes a method of harvesting a non-vascular photosynthetic organism (NVPO) from an aqueous culture by mixing an effective amount of a polymer flocculant and/or coagulant with the aqueous culture to cause sedimentation (settling) or increase the rate of sedimenation, and collecting the NVPO, where the method does not involve dissolved air.

In another aspect, the present disclosure includes a method of harvesting a non-vascular photosynthetic organism (NVPO) from an aqueous culture by mixing an effective amount of a polymer flocculant with the aqueous culture directly in a tank without involving dissolved air, and collecting the flocs of the NVPO.

The aforementioned methods of harvesting may also be performed in a pond where NVPO grows. In one aspect, the present disclosure includes an in-pond method of harvesting a non-vascular photosynthetic organism (NVPO) from an aqueous culture in a pond where the NVPO grows, the method comprises mixing an effective amount of a flocculant with the aqueous culture in a pond to form flocs of the NVPO; and collecting the flocs of the NVPO.

A method of the present disclosure can be used to harvest a NVPO from any types of ponds. In one aspect, a method of the present disclosure is performed is a natural pond. In another aspect, a method of the present disclosure is performed is artificial pond. In another aspect, a method of the present disclosure is performed is artificial pond having a depth of at least 6 inches.

A method of the present disclosure can be used to harvest a NVPO from any size of pond. In one aspect, a method of the present disclosure can be used to harvest a NVPO from a pond of at least 5 square meters, at least 10 square meters, at least 200 square meters, at least 500 square meters, at least 1,500 square meters, at least 2,500 square meters, in area, or greater.

In one aspect, a method of the present disclosure can be used to harvest a NVPO from a pond having a volume of selected from the group consisting of at least 20,000 gallons, at least 40,000 gallons, at least 80,000 gallons, at least 100,000 gallons, at least 150,000 gallons, at least 200,000 gallons, at least 250,000 gallons, at least 500,000 gallons, at least 600,000 gallons, at least 800,000 gallons, at least 1,000,000 gallons, at least 2,000,000 gallons, at least 3,000,000 gallons, at least 4,000,000 gallons, at least 5,000,000 gallons, and at least 6,000,000 gallons.

In another aspect, pond volume may be selected from the group consisting of between 20,000 and 6,000,000 gallons, between 40,000 and 6,000,000 gallons, between 80,000 and 6,000,000 gallons, between 100,000 and 6,000,000 gallons, between 150,000 and 6,000,000 gallons, between 200,000 and 6,000,000 gallons, between 250,000 and 6,000,000 gallons, between 500,000 and 6,000,000 gallons, between 600,000 and 6,000,000 gallons, between 800,000 and 6,000,000 gallons, between 1,000,000 and 6,000,000 gallons, between 2,000,000 and 6,000,000 gallons, between 3,000,000 and 6,000,000 gallons, between 4,000,000 and 6,000,000 gallons, and between 5,000,000 and 6,000,000 gallons.

In another aspect, pond volume may be selected from the group consisting of between 20,000 and 2,000,000 gallons, between 40,000 and 2,000,000 gallons, between 80,000 and 2,000,000 gallons, between 100,000 and 2,000,000 gallons, between 150,000 and 2,000,000 gallons, between 200,000 and 2,000,000 gallons, between 250,000 and 2,000,000 gallons, between 500,000 and 2,000,000 gallons, between 600,000 and 2,000,000 gallons, between 800,000 and 2,000,000 gallons, between 1,000,000 and 2,000,000 gallons, between 2,000,000 and 3,000,000 gallons, between 3,000,000 and 4,000,000 gallons, between 4,000,000 and 5,000,000 gallons, and between 5,000,000 and 6,000,000 gallons.

The methods of harvesting NVPO described herein can be achieve using sedimentation and in one particular embodiment by the use of clarifiers. The use of clairifiers is well established in the waste water treatment industry. In this embodiment, a non-vascular photosynthetic organism (NVPO) from an aqueous culture is mixed with an effective amount of a polymer flocculant and/or coagulant with the aqueous culture to cause sedimentation (settling) or to increase the rate of settling (sedimentation) of the NVPO, and collecting the sedimented NVPO. In a clarifier, material is introduced at the bottom of the holding tank and exits by means of a weir at the top. The clarifier is configured so that there is sufficient residence time of the aqueous culture to allow the NVPO to settle to collection area.

A common type of clarifier is the mechanical circular clarifier. Circular clarifiers typically are conical in share and are classified as either center feed or peripheral feed depending on where the material to be clarified enters the unit. Parallel plate clarifiers are also known in the industry. Parallel plate clarifiers contain a series of inclined parallel plates. The settling area of a parallel plate clarifier is the total of the areas projected on the horizontal plane by the plates. The clarifier is sized to accommodate the anticipated flow rate of aqueous culture medium. Industrial size clarifiers can be obtained from a number of sources.

One of ordinary skill in the art understands that each of the various aspects described above applies to any methods of the present disclosure, including, without limitation, harvesting a NVPO with the aid of dissolved air flotation, harvesting a NVPO without the aid of dissolved air flotation, and harvesting a NVPO from a pond directly. One of ordinary skill in the art also understands that steps of a method of the present disclosure may be performed in any order.

The methods of the present disclosure produces flocs of a NVPO. These flocs of a NVPO constitute a biomass slurry. As used herein, “biomass” is organic or biological material that is derived from living, or recently living biological organisms. Biomass may comprise intact organisms, debris of organisms, or both. As used herein, a “biomass slurry” relates to a mixture of biomass and comprises heterogeneous, multi-phase material with complex physical and chemical properties.

The present disclosure includes a biomass slurry comprising a non-vascular photosynthetic organism (NVPO) at a concentration of at least 0.2% and a polymer flocculant, wherein the ratio between the weight of the polymer flocculant and the Ash-Free Dry Weight (AFDW) of the NVPO is at most 1.5%.

In one aspect, the biomass slurry of the present disclosure comprises a NVPO at a concentration selected from the group consisting of at least 0.3%, at least 0.4%, at least 0.5%, at least 0.6%, at least 0.7%, at least 0.8%, at least 0.9%, at least 1.0%, at least 1.5%, at least 2.0%, at least 2.5%, at least 3.0%/0, at least 3.5%, at least 4.0%, at least 4.5%, at least 5%, at least 5.5%, at least 6%, at least 6.5%, at least 7%, at least 7.5%, at least 8%, at least 8.5%, at least 9%, at least 9.5%, at least 10%, at least 11%, at least 12%, at least 13%, at least 14%, at least 15%, at least 20%, at least 30%, at least 40%, and at least 50%.

In another aspect, the biomass slurry of the present disclosure comprises a NVPO at a concentration selected from the group consisting of at least 1.0%, at least 1.5%, at least 2.0%, at least 2.5%, at least 3.0%, at least 3.5%, at least 4.0%, at least 4.5%, at least 5%, at least 5.5%, at least 6%, at least 6.5%, at least 7%, at least 7.5%, at least 8%, at least 8.5%, at least 9%, at least 9.5%, at least 10%, at least 11%, at least 12%, at least 13%, at least 14%, at least 15%, at least 20%, at least 30%, at least 40%, and at least 50%.

In one aspect, the biomass slurry of the present disclosure comprises a NVPO at a concentration selected from the group consisting of at least 3.0%, at least 3.5%, at least 4.0%, at least 4.5%, at least 5%, at least 5.5%, at least 6%, at least 6.5%, at least 7%, at least 7.5%, at least 8%, at least 8.5%, at least 9%, at least 9.5%, at least 10%, at least 11%, at least 12%, at least 13%, at least 14%, at least 15%, at least 20%, at least 30%, at least 40%, and at least 50%.

In one aspect, the biomass slurry of the present disclosure comprises a NVPO at a concentration selected from the group consisting of at least 5%, at least 5.5%, at least 6%, at least 6.5%, at least 7%, at least 7.5%, at least 8%, at least 8.5%, at least 9%, at least 9.5%, at least 10%, at least 11%, at least 12%, at least 13%, at least 14%, at least 15%, at least 20%, at least 30%, at least 40%, and at least 50%.

In one aspect, the biomass slurry of the present disclosure comprises a NVPO at a concentration selected from the group consisting of at least 7.5%, at least 8%, at least 8.5%, at least 9%, at least 9.5%, at least 10%, at least 11%, at least 12%, at least 13%, at least 14%, at least 15%, at least 20%, at least 30%, at least 40%, and at least 50%.

In one aspect, the biomass slurry of the present disclosure comprises a ratio between the weight of the polymer flocculant and the Ash-Free Dry Weight (AFDW) of the NVPO selected from the group consisting of at most 1.3%, at most 1.2%, at most 1.1%, at most 1.0%, at most 0.9%, at most 0.8%, at most 0.7%, at most 0.6%, at most 0.5%, at most 0.4%, at most 0.3%, at most 0.2%, and at most 0.1%.

In another aspect, the biomass slurry of the present disclosure comprises a ratio between the weight of the polymer flocculant and the Ash-Free Dry Weight (AFDW) of the NVPO selected from the group consisting of at most 1.1%, at most 1.0%, at most 0.9%, at most 0.8%, at most 0.7%, at most 0.6%, at most 0.5%, at most 0.4%, at most 0.3%, at most 0.2%, and at most 0.1%.

In another aspect, the biomass slurry of the present disclosure comprises a ratio between the weight of the polymer flocculant and the Ash-Free Dry Weight (AFDW) of the NVPO selected from the group consisting of at most 0.9%, at most 0.8%, at most 0.7%, at most 0.6%, at most 0.5%, at most 0.4%, at most 0.3%, at most 0.2%, and at most 0.1%.

In another aspect, the biomass slurry of the present disclosure comprises a ratio between the weight of the polymer flocculant and the Ash-Free Dry Weight (AFDW) of the NVPO selected from the group consisting of at most 0.7%, at most 0.6%, at most 0.5%, at most 0.4%, at most 0.3%, at most 0.2%, and at most 0.1%.

In another aspect, the biomass slurry of the present disclosure comprises a ratio between the weight of the polymer flocculant and the Ash-Free Dry Weight (AFDW) of the NVPO selected from the group consisting of at most 0.5%, at most 0.4%, at most 0.3%, at most 0.2%, and at most 0.1%.

In another aspect, the biomass slurry of the present disclosure comprises a ratio between the weight of the polymer flocculant and the Ash-Free Dry Weight (AFDW) of the NVPO selected from the group consisting of at most 0.3%, at most 0.2%, and at most 0.1%.

In one aspect, the biomass slurry of the present disclosure comprises a water content of less than 95%.

In another aspect, the biomass slurry of the present disclosure comprises a water content selected from the group consisting of less than 95%, less than 90%, less than 80%, less than 70%, less than 60%, less than 50%, less than 40%, less than 30%, less than 20%, less than 10%, and less than 5%.

In another aspect, the biomass slurry of the present disclosure comprises a water content above 50% and less than selected from the group consisting of 99%, 98%, 97%, 95%, 94%, 93%, 92%, 91%, 90%, 89%, 88%, 87%, 86%, 85%, 84%, 80%, 70%, and 60%.

In another aspect, the biomass slurry of the present disclosure comprises a water content selected from the group consisting of less than 50%, less than 40%, less than 30%, less than 20%, less than 10%, and less than 50/%.

In another aspect, the biomass slurry of the present disclosure comprises a water content selected from the group consisting of less than 30%, less than 20%, less than 10%, and less than 5%.

In one aspect, the biomass slurry of the present disclosure comprises an aqueous environment with a TDS of less than 1500 mg/L. In another aspect, the biomass slurry of the present disclosure comprises an aqueous environment with a TDS from 1500 to 35000 mg/L. In another aspect, the biomass slurry of the present disclosure comprises an aqueous environment with a TDS of greater than 35000 mg/L. In another aspect, the biomass slurry of the present disclosure comprises an aqueous environment with a TDS over 100000 mg/L. In another aspect, the biomass slurry of the present disclosure comprises an aqueous environment with a TDS of at least 1500, 2000, 2500, 3000, 3500, 4000, 4500, 5000, 5500, 6000, 6500, 7000, 7500, 8000, 8500, 9000, 10000, 10500, 1000, 11500, 12000, 12500, 13000, 13500, 14000, 14500, 15000, 16000, 16500, 17000, 17500, 18000, 18500, 19000, 20000, 20500, 22000, 22500, 22000, 22500, 23000, 23500, 24000, 24500, 25000, 26000, 26500, 27000, 27500, 28000, 28500, 29000, 30000, 30500, 31000, 33500, 32000, 32500, 33000, 33500, 34000, 34500, 35000, 36000, 36500, 37000, 37500, 38000, 38500, 39000, 40000, 40500, 41000, 41500, 42000, 42500, 43000, 43500, 44000, 44500, 45000, 46000, 46500, 47000, 47500, 48000, 48500, 49000 mg/L.

In one aspect, the biomass slurry of the present disclosure comprises an aqueous environment with a pH value between 1 and 13. In another aspect, the biomass slurry of the present disclosure comprises an aqueous environment with a pH value selected from the group consisting of between 2 and 13, between 3 and 13, between 4 and 13, between 5 and 13, between 6 and 13, between 7 and 13, between 8 and 13, between 9 and 13, between 10 and 13, between 11 and 13, between 12 and 13, between 2 and 12, between 3 and 12, between 4 and 12, between 5 and 12, between 6 and 12, between 7 and 12, between 8 and 12, between 9 and 12, between 10 and 12, between 11 and 12, between 2 and 11, between 3 and 11, between 4 and 11, between 5 and 11, between 6 and 11, between 7 and 11, between 8 and 11, between 9 and 11, between 10 and 11, between 2 and 10, between 3 and 10, between 4 and 10, between 5 and 10, between 6 and 10, between 7 and 10, between 8 and 10, between 9 and 10, between 2 and 9, between 3 and 9, between 4 and 9, between 5 and 9, between 6 and 9, between 7 and 9, between 8 and 9, between 2 and 8, between 3 and 8, between 4 and 8, between 5 and 8, between 6 and 8, between 7 and 8, between 2 and 7, between 3 and 7, between 4 and 7, between 5 and 7, between 6 and 7, between 2 and 6, between 3 and 6, between 4 and 6, between 5 and 6, between 2 and 5, between 3 and 5, between 4 and 5, between 2 and 4, between 3 and 4, and between 2 and 3. In another aspect, the biomass slurry of the present disclosure comprises an aqueous environment with a pH value between 7.80 and 9.34.

The present disclosure also includes an aqueous culture comprising a non-vascular photosynthetic organism (NVPO) at a concentration between 0.001% and 0.2%, and a polymer flocculant at a concentration of less than 10 parts per million (ppm). An aqueous culture of the present disclosure may comprise a total dissolved solids (TDS) of any level. In one aspect, an aqueous culture of the present disclosure is in brackish water having a TDS selected from the group consisting of between 1500 and 35000, between 5000 and 35000, and between 15000 and 35000 mg/L.

In one aspect, the aqueous culture of the present disclosure comprises an aqueous environment with a TDS of less than 1500 mg/L. In another aspect, the aqueous culture of the present disclosure comprises an aqueous environment with a TDS from 1500 to 35000 mg/L. In another aspect, the aqueous culture of the present disclosure comprises an aqueous environment with a TDS of greater than 35000 mg/L. In another aspect, the aqueous culture of the present disclosure comprises an aqueous environment with a TDS over 100000 mg/L. In another aspect, the aqueous culture of the present disclosure comprises an aqueous environment with a TDS of at least 1500, 2000, 2500, 3000, 3500, 4000, 4500, 5000, 5500, 6000, 6500, 7000, 7500, 8000, 8500, 9000, 10000, 10500, 11000, 11500, 12000, 12500, 13000, 13500, 14000, 14500, 15000, 16000, 16500, 17000, 17500, 18000, 18500, 19000, 20000, 20500, 22000, 22500, 22000, 22500, 23000, 23500, 24000, 24500, 25000, 26000, 26500, 27000, 27500, 28000, 28500, 29000, 30000, 30500, 31000, 33500, 32000, 32500, 33000, 33500, 34000, 34500, 35000, 36000, 36500, 37000, 37500, 38000, 38500, 39000, 40000, 40500, 41000, 41500, 42000, 42500, 43000, 43500, 44000, 44500, 45000, 46000, 46500, 47000, 47500, 48000, 48500, 49000 mg/L.

In one aspect, the aqueous culture of the present disclosure comprises an aqueous environment with a pH value between 1 and 13. In another aspect, the aqueous culture of the present disclosure comprises an aqueous environment with a pH value selected from the group consisting of between 2 and 13, between 3 and 13, between 4 and 13, between 5 and 13, between 6 and 13, between 7 and 13, between 8 and 13, between 9 and 13, between 10 and 13, between 11 and 13, between 12 and 13, between 2 and 12, between 3 and 12, between 4 and 12, between 5 and 12, between 6 and 12, between 7 and 12, between 8 and 12, between 9 and 12, between 10 and 12, between 11 and 12, between 2 and 1, between 3 and 11, between 4 and 11, between 5 and 11, between 6 and 11, between 7 and 11, between 8 and 11, between 9 and 11, between 10 and 11, between 2 and 10, between 3 and 10, between 4 and 10, between 5 and 10, between 6 and 10, between 7 and 10, between 8 and 10, between 9 and 10, between 2 and 9, between 3 and 9, between 4 and 9, between 5 and 9, between 6 and 9, between 7 and 9, between 8 and 9, between 2 and 8, between 3 and 8, between 4 and 8, between 5 and 8, between 6 and 8, between 7 and 8, between 2 and 7, between 3 and 7, between 4 and 7, between 5 and 7, between 6 and 7, between 2 and 6, between 3 and 6, between 4 and 6, between 5 and 6, between 2 and 5, between 3 and 5, between 4 and 5, between 2 and 4, between 3 and 4, and between 2 and 3. In another aspect, the aqueous culture of the present disclosure comprises an aqueous environment with a pH value between 7.80 and 9.34.

In one aspect, the aqueous culture of the present disclosure comprises the NVPO at a concentration of between 0.001% and 0.2%, measured by weight/volume (Ash Free Dry Weight of the NVPO/culture volume). In another aspect, the aqueous culture of the present disclosure comprises the NVPO at a concentration selected from the group consisting of between 0.001% and 0.2%, between 0.005% and 0.2%, between 0.01% and 0.2%, between 0.02% and 0.2%, between 0.03% and 0.2%, between 0.04% and 0.2%, between 0.05% and 0.2%, between 0.06% and 0.2%, between 0.07% and 0.2%, between 0.08% and 0.2%, between 0.09% and 0.2%, between 0.1% and 0.2%, between 0.15% and 0.2%, between 0.001% and 0.15%, between 0.001% and 0.1%, between 0.001% and 0.09%, between 0.001% and 0.08%, between 0.001% and 0.07%, between 0.001% and 0.06%, between 0.001% and 0.05%, between 0.001% and 0.04%, between 0.001% and 0.03%, between 0.001% and 0.02%, between 0.001% and 0.01%, between 0.001% and 0.005%, between 0.001% and 0.002%, between 0.003% and 0.15%, between 0.005% and 0.125%, between 0.01% and 0.1%, between 0.02% and 0.08%, and between 0.04% and 0.06%.

In one aspect, the aqueous culture of the present disclosure comprises a polymer flocculant at a concentration selected from the group consisting of greater than 0, but less than 10 ppm, less than 9 ppm, less than 8 ppm, less than 7 ppm, less than 6 ppm, less than 5 ppm, less than 4 ppm, less than 3 ppm, less than 2 ppm, less than 1 ppm, and less than 0.5 ppm.

In another aspect, the aqueous culture of the present disclosure comprises a polymer flocculant at a concentration selected from the group consisting of greater than 0, but less than 8 ppm, less than 7 ppm, less than 6 ppm, less than 5 ppm, less than 4 ppm, less than 3 ppm, less than 2 ppm, less than 1 ppm, and less than 0.5 ppm. In another aspect, the aqueous culture of the present disclosure comprises a polymer flocculant at a concentration selected from the group consisting of greater than 0, but less than 6 ppm, less than 5 ppm, less than 4 ppm, less than 3 ppm, less than 2 ppm, less than 1 ppm, and less than 0.5 ppm. In another aspect, the aqueous culture of the present disclosure comprises a polymer flocculant at a concentration selected from the group consisting of greater than 0, but less than 6 ppm, less than 5 ppm, less than 4 ppm, less than 3 ppm, less than 2 ppm, less than 1 ppm, and less than 0.5 ppm. In another aspect, the aqueous culture of the present disclosure comprises a polymer flocculant at a concentration selected from the group consisting of greater than 0, but less than 4 ppm, less than 3 ppm, less than 2 ppm, less than 1 ppm, and less than 0.5 ppm. In another aspect, the aqueous culture of the present disclosure comprises a polymer flocculant at a concentration selected from the group consisting of greater than 0, but less than 2 ppm, less than 1 ppm, and less than 0.5 ppm.

Following the methods of the present disclosure, upon collection of NVPO flocs from a flocculated aqueous culture, the remaining aqueous medium may comprise a non-vascular photosynthetic organism (NVPO) at a concentration between 0.0001% and 0.02%, and a polymer flocculant at a concentration of less than 1 parts per million (ppm). In the case of a DAF process, the remaining aqueous medium is also called subnatant.

In one aspect, the polymer flocculant present in a remaining aqueous medium has no significant impacts on the growth of a to-be-harvested NVPO. In another aspect, the polymer flocculant present in a remaining aqueous medium has no significant impacts on pH of the aqueous culture. In a further aspect, the polymer flocculant present in a remaining aqueous medium has no significant impacts on the level of nitrate or phosphate in the aqueous culture.

In one aspect, the NVPO concentration in the remaining aqueous medium may be selected from the group consisting of between 0.0001% and 0.02%, between 0.0005% and 0.02%, between 0.001% and 0.02%, between 0.002% and 0.02%, between 0.003% and 0.02%, between 0.004% and 0.02%, between 0.005% and 0.02%, between 0.006% and 0.02%, between 0.007% and 0.02%, between 0.008% and 0.02%, between 0.009% and 0.02%, between 0.01% and 0.02%, between 0.015% and 0.02%, between 0.0001% and 0.015%, between 0.0001% and 0.01%, between 0.0001% and 0.009%, between 0.0001% and 0.008%, between 0.0001% and 0.007%, between 0.0001% and 0.006%, between 0.0001% and 0.005%, between 0.0001%0 and 0.004%, between 0.0001% and 0.003%, between 0.0001% and 0.002%, between 0.0001% and 0.001%, between 0.0001% and 0.0005%, between 0.0001% and 0.0002%, between 0.0003% and 0.015%, between 0.0005% and 0.0125%, between 0.001% and 0.01%, between 0.002% and 0.008%, and between 0.004% and 0.006%.

In one aspect, the polymer concentration in the remaining aqueous medium may be less than selected from the group consisting of 1, 0.9, 0.8, 0.7, 0.6, 0.5, 0.4, 0.3, 0.2, 0.1, 0.05, 0.01, 0.005, and 0.001 ppm. Any polymer concentration of the present disclosure can be measured and quantified using the method provided in Example 6.

An aqueous medium derived from a method of the present disclosure may be measured and characterized by its turbidity. As used herein, “turbidity” relates to a measure of the degree to which the water loses its transparency due to the presence of suspended particulates. The more total suspended solids in the water, the murkier it seems and the higher the turbidity. Turbidity of an aqueous medium may be measured in Nephelometric Turbidity Units (NTU), which provides a good correlation with the concentration of particles in an aqueous medium that affect clarity.

In one aspect, an aqueous medium of the present disclosure has a turbidity above 0.01 and less than selected from the group consisting of 1, 2, 3, 4, 5, 6, 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 100, 150, 200, 300, 400, 500, 600, 700, 800, 900, 1000, 1500, and 2000 NTU. In another aspect, an aqueous medium of the present disclosure has a turbidity selected from the group consisting of between 1 and 10, between 2 and 11, between 3 and 12, between 4 and 13, between 5 and 14, between 6 and 15, between 7 and 16, between 8 and 17, between 9 and 18, between 10 and 19, between 11 and 20, between 12 and 21, between 13 and 22, between 14 and 23, between 15 and 24, between 16 and 25, between 17 and 26, between 18 and 27, between 19 and 28, between 20 and 29, between 21 and 30, between 22 and 31, between 23 and 32, between 24 and 33, between 25 and 34, between 26 and 35, between 27 and 36, between 28 and 37, between 29 and 38, between 30 and 39, between 31 and 40, between 32 and 41, between 33 and 42, between 34 and 43, between 35 and 44, between 36 and 45, between 37 and 46, between 38 and 47, between 39 and 48, between 40 and 49, between 45 and 54, between 50 and 59, between 55 and 64, between 60 and 69, between 65 and 74, between 70 and 79, between 75 and 84, between 80 and 89, between 85 and 94, and between 90 and 99 NTU.

### EXAMPLES

### Example 1

### Harvest of Microalgae by a Dissolved Air Flotation (DAF) Process

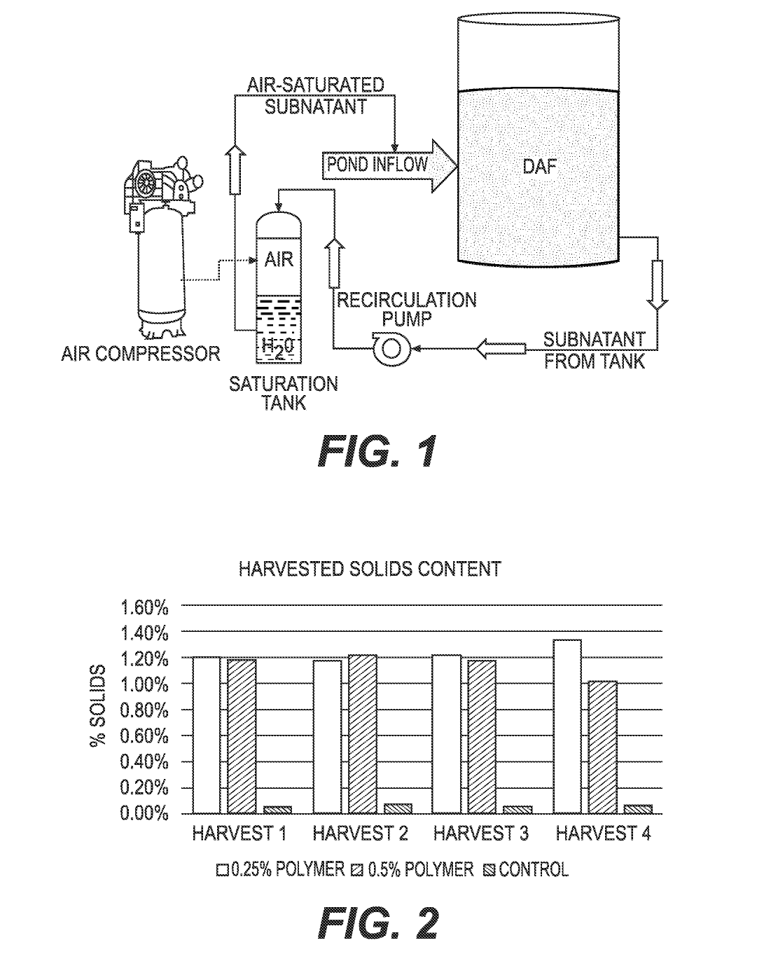

A dissolved air flotation (DAF) process is employed to harvest/dewater spirulina algae (SE50359) from aqueous media originating from outdoor raceway ponds. The DAF system (FIG. 1) utilizes air-saturated recycled media to generate air bubbles, which bind and carry flocculated algal cells to the media surface for removal. Specifically, a 40 gpm WWW DAF system is used. In this system, air-saturated recycled subnatant is generated in a saturation tank with a pressure of 50-60 psi. Input of algae culture is first mixed with polymer at a rate proportional to a desired dosing and stock concentration of polymer. This mixed input is then blended with the air-saturated recycled subnatant as it flows into the DAF structure. When the inflow enters the DAF structure, a pressure decrease causes air bubbles to come out of the solution and float towards the surface. As the air bubbles ascend, they clinge to flocs of biomass and float them to the surface where flocs are harvested. Upon harvesting of the biomass flocs, the resulting subnatant is returned to a pond for further algae growth.

### Example 2

### Air-Flotation-Free Harvest of Microalgae

Polymer flocculant 859BS flocculates spirulina effectively in lab performed jar tests. In these jar tests, flocculated spirulina cells float to the medium surface immediately and automatically without the aid of air flotation. These tests can achieve a biomass removal efficiency of approximately 90%, with an 859BS concentration as low as 0.1 ppm. However, when polymer flocculant 859BS is used in a dissolved air flotation (DAF) process to harvest spirulina from cultures grown in raceway ponds, a lower efficiency is achieved.

An efficiency drop can be due to the fragility of the spirulina algae polymer bond. Exposure to shear forces applied in the pumping, mixing, recirculation, and dissolved air addition processes can also cause the flocculated spirulina algae to break up. To reduce the disruptive shear forces, a modified DAF process is used. A modified process includes removing dissolved air, skipping recirculation of media, and injecting polymer directly into the DAF reactor vessel (as opposed to pre-vessel polymer addition in a standard DAF process). A comparison of average harvesting efficiency using dissolved air or no air harvests is shown in Table 1. Process improvements include:
1) an improvement of dewatering efficiency (approximately 80%-90% biomass harvesting efficiency compared with approximately 50% before modification),
2) elimination of problematic foam generation, which in turn improves the accuracy of measurements with harvested spirulina cells,
3) an increase of harvested solids content, and
4) elimination of specific expenditures (anti-foam solutions, recirculation pumps, air compressors, etc.).

TABLE 1
A comparison of average flocculation efficiency using dissolved
air or no air harvests. Efficiency is calculated based on
optical density measurement data at 560 nm from two hour
harvests. 859BS stock concentration of 0.25% was used.

| | Average Harvest |
|---|---|
| DAF System Parameters | Efficiency |
| No air, no media recirculation, internal DAF | 89.95% |
| injection position | |
| No air, no media recirculation, standard polymer | 76.99% |
| injection position | |
| Air, media recirculation, standard polymer | 54.41% |
| injection position | |

### Example 3

### In-Pond Harvest of Microalgae

Tests are performed using spirulina with a goal to harvest biomass directly from a pond while using the same levels of polymer flocculant as a DAF harvest process. Varying amounts of polymer 859BS stock solutions at a concentration of 0.25% or 0.5% are added to a pond of spirulina culture at a density of approximately 0.5 g/L (OD of 0.5 at 560 nm). Mixing of the culture is achieved using a paddlewheel. The final polymer dosing is 5 ppm. The culture media have a pH of 10 and a total dissolved solids (TDS) of 15,000 mg/L.

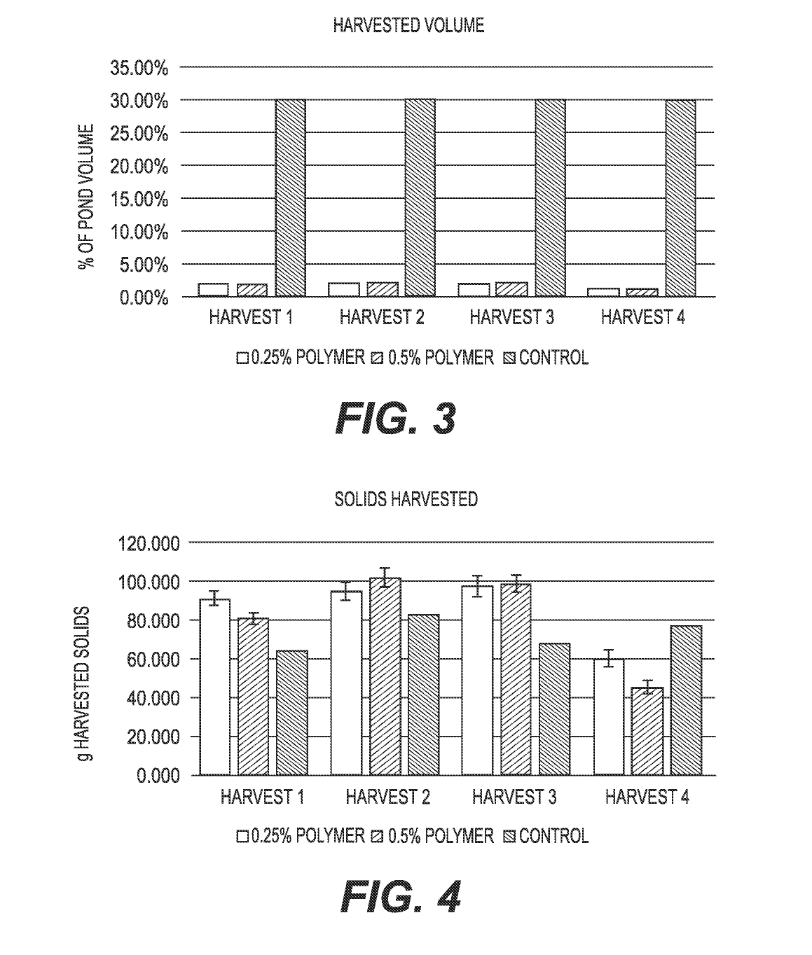

As polymer is added, spirulina cells flocculate. Flocculated cells float to the top of the culture, and are collected at a static collection weir. Four runs of harvest are performed every 2-3 days. Both harvest solids content and harvest volume are recorded for each run, along with a control harvest using a DAF process (see Example 1). Compared to the control harvest, polymer assisted harvesting achieves increases in harvest solids content and decreases in harvest volume. For example, harvest solids content increases to 1.23% from an average of 0.06% in controls, which reflects a more than 20 times increase (FIG. 2). Meanwhile, the volume of harvest decreases to an average of 1.7% pond volume in polymer-assisted runs, as compared to a 30% pond volume in controls. This reflects a more than 17 fold reduction (FIG. 3).

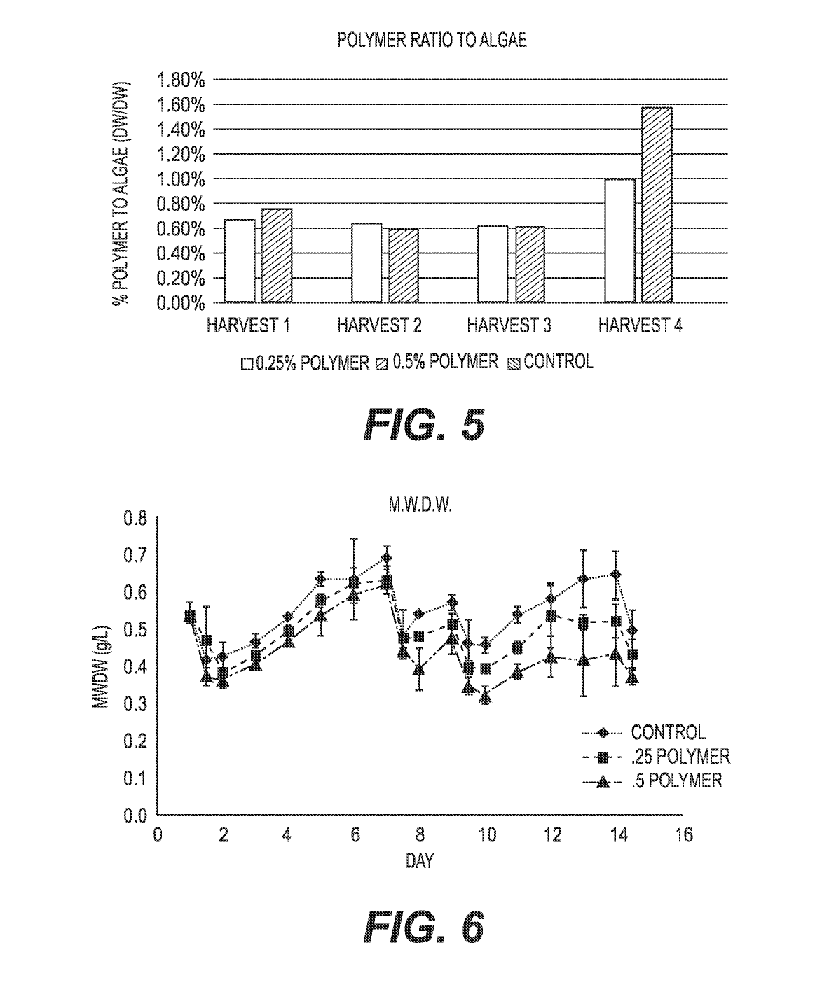

In-pond harvest achieves higher solids removal than controls in the first three runs, though this trend reverses by the fourth run (FIG. 4). All four runs maintained a polymer to algae ratio below 1.5% (FIG. 5).

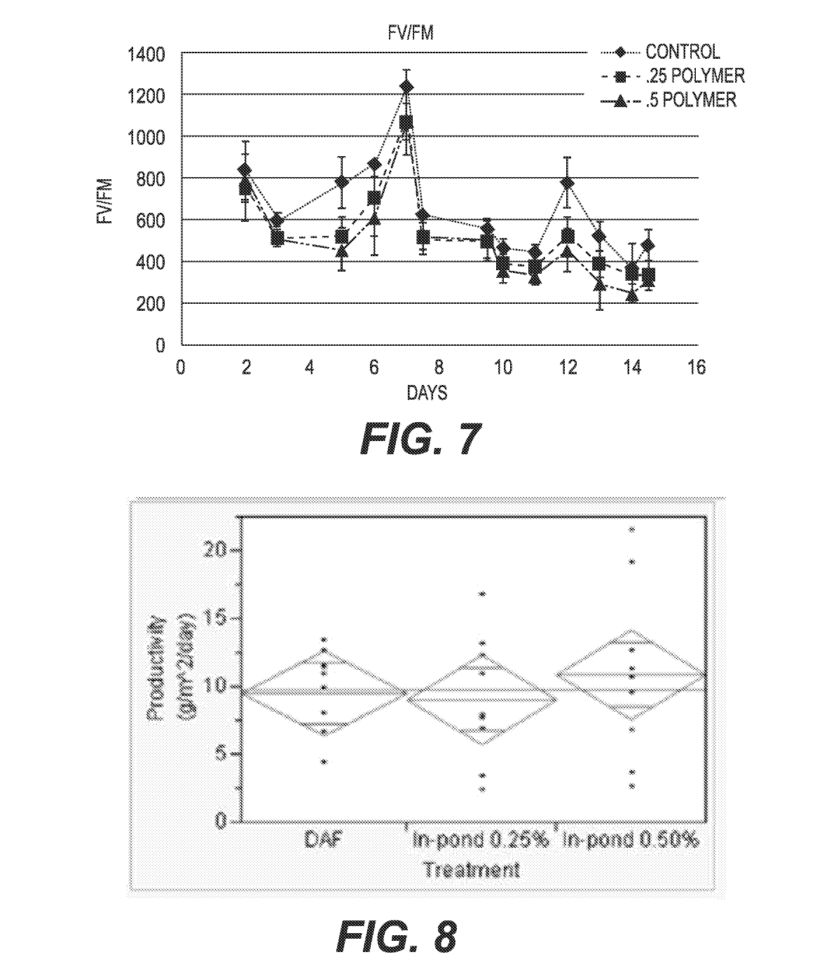

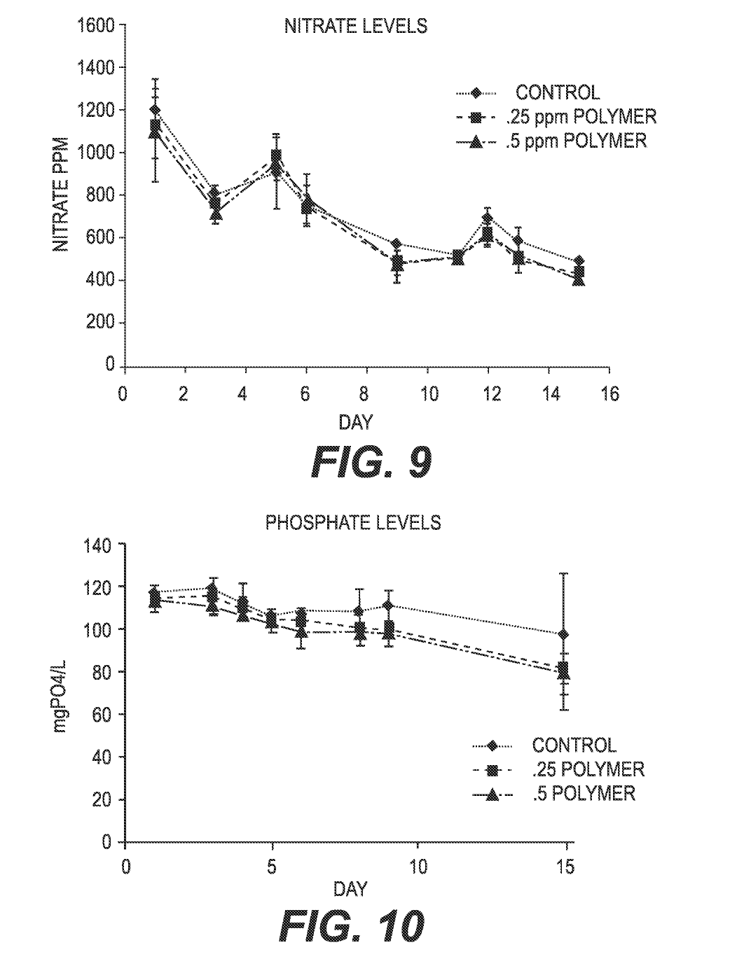

In-pond application of polymer is also evaluated for its impacts on algae strain performance. Visible effects on strain performance are observed after fourth polymer-assisted harvest (FIG. 6 and FIG. 7). More biomass is harvested by in-pond polymer application than expected compared to the control. No difference is observed for the average daily production between in-pond polymer-assisted harvesting and a control harvest using a DAF process (FIG. 8). Impacts from the in-pond polymer application on a culture's nutrient levels are also monitored. No significant impacts are observed on nitrate levels (FIG. 9). Phosphate level decreases in the media, though this phosphate decrease likely is not associated with biomass productivity (FIG. 10).

### In-Pond Harvest of Microalgae in Pond P08

A pilot run of in-pond, polymer-assisted harvest of microalgae is performed in pond P08. The culture medium in P08 has a pH of 9-10 and a TDS of 15,000 mg/L. Approximately 30% of the pond volume is dosed with polymer 859BS at 15 ppm (550 g polymer, polymer stock concentration of 0.25%). Pond flow rate is set at 4,347 Gallon Per Minute (GPM). Polymer flow rate of 9.2 GPM is used. The pond has a starting culture density of 1.03 g/L, and is left with a final culture density of 0.744 g/L upon harvesting. Approximately 28.28% of the biomass from the pond is harvested. The harvested float has a volume of 2,090 L (approximately 1.8% of the pond volume) with a solids content of 1.14%.

### Example 4

### Methods to Determine Polymer Effectiveness

Two methods, one indirect and one direct, are employed to evaluate flocculation efficiency of a polymer and the optimal dosing of a polymer. The indirect method is carried out by sampling the influent stream (pond or initial culture) of algae prior to any addition of polymer and measuring the Ash Free Dry Weight (AFDW). Once polymer is added and allowed to flocculate, subnatant is sampled from the stream coming off of the DAF system or jar test and the AFDW is measured. Subnatant is recycled/processed before returning to ponds and represents the algae that have not been bound to polymer. When calculating an indirect efficiency, the ratio of influent to subnatant is taken to determine the amount of algae that is extracted from the stream. The following formula is used to calculate an indirect efficiency:

1−(Subnatant AFDW/Influent AFDW)=% Efficiency.

The second method is applied when the volume of input algae culture is measured. The influent stream of algae is sampled prior to any addition of flocculant and the AFDW is measured. The volume of product harvested off the DAF (dewatered algae at ˜80% moisture) is compared to the total culture volume processed through the system. In this method, a graduated cylinder or appropriate sized graduate container for accurate measurement of harvested product is used. When a predetermined volume of pond has been harvested the product recovered from the DAF is sampled and an AFDW is recorded for the sample. Samples are well mixed before sampling to prevent an inhomogeneous sampling. During the mixing process the breaking up of flocs is permitted. The following formula is used to calculate a direct efficiency:

(Volume product*AFDW Product)/(Volume processed*AFDW influent)=% Efficiency

### Example 5

### Polymer Flocculant and Coagulant Screening

### Sources of Tested Polymers and Preparation of Stock Solutions

One hundred twenty five polymers from three manufactures Monolyte, Nalco, and Ashland are screened for their flocculation effectiveness (Table 2 to Table 11). Each polymer varies in molecular weight, charge, and charge density. Both emulsion and dry based polymers are tested. Polymer is made up in batches so that small volumes of polymer could be made to dose small volumes of algae and quickly determine the polymers' effectiveness. The stock concentration of each polymer is set at 0.25%. This stock concentration proves to be the best balance between viscosity and active polymer density.

For dry polymers, 0.5 g of polymer is added to 200 mL of deionized H₂O (DiH₂O) to make up a stock that could then be dosed at the desired mg polymer/L algae. A magnetic stirrer is used in a beaker with the DiH₂O and spun at 200-400 rpm so that a vortex is formed. Polymer is slowly added to ensure that it is mixed into the water and properly wetted.

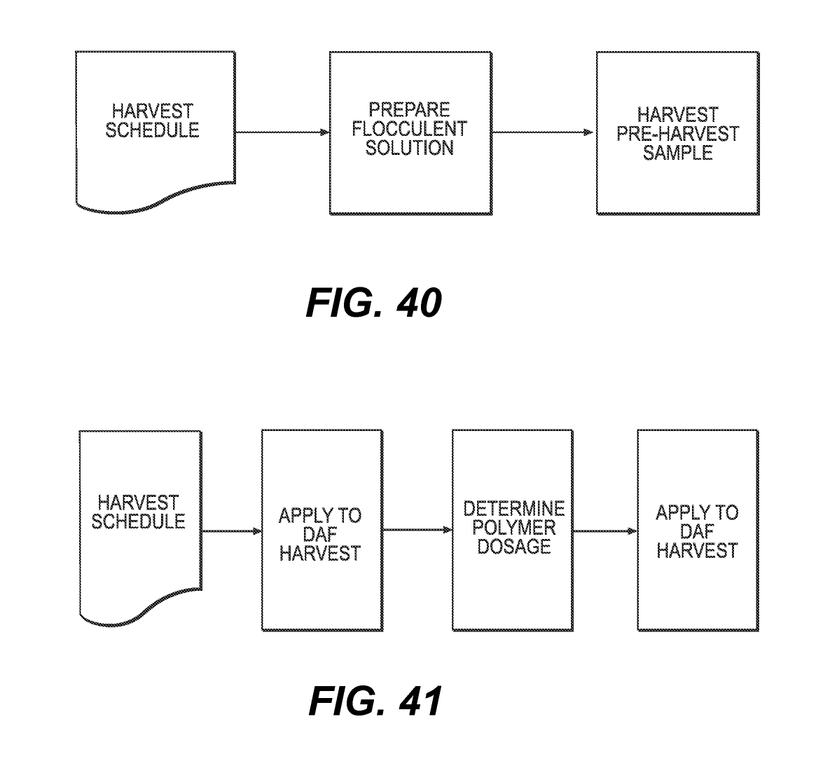

Emulsion polymer stocks are made using the same method as above except that measurements are made volumetrically (e.g., adding 200 uL polymer to 200 mL DiH₂O). The amount of active polymer is calculated based on the specific gravity to get the exact concentration of active material in the ˜0.25% stock solution. Procedures for preparing a manual batch of polymer flocculant stock solution used in a jar-test setting are described in FIG. 40.

TABLE 2
Total number of polymers screened
from Nalco, Ashland, and Monolyte.

| | | Polymers | Number of | Test | |
|---|---|---|---|---|---|
| Company | Type | Tested | Tests | Format | |
| Ashland | Cationic Polymers | 44 | 131 | 50 | mL |
| | Cationic | 9 | 27 | 50 | mL |
| | Coagulants | | | | |
| | Combinations | 9 | 45 | 50 | mL |
| Nalco | Cationic Polymers | 32 | 97 | 50 | mL |
| | Anionic Polymers | 18 | 6 | 50 | mL |
| | Cationic | 15 | 15 | 50 | mL |
| | Coagulants | | | | |
| | Combinations | 6 | 25 | 50 | mL |
| Monolyte | Cationic Polymers | 2 | 21 | 1 | L |
| | Colloidal | 2 | 8 | 1 | L |
| | Destabilizesr | | | | |

TABLE 3
A list of Monolyte polymers and Colloidal Destabilizers tested.

| | Type |
|---|---|
| CE-3390 | Cationic |
| AE-2992 | Anionic |
| CD 1 | Colloidal Destabilizer (Aluminum Based) |
| CD 2 | Colloidal Destabilizer (Ferric based) |

TABLE 4
A list of Nalco cationic and Anionic polymer flocculants tested and their properties.

| | | | % | | % | | | |
|---|---|---|---|---|---|---|---|---|
| Product | | | Mole | Molecular | Active | | | |
| Names | Form | Charge | Charge | Weight | Polymer | S.G. | NSF | Other |
| 7190 PLUS | Latex | Cationic | 1 | V. High | 26 | 1.06 | NSF | |
| 7193 PLUS | Latex | Cationic | 3 | V. High | 26 | 1.04 | | |
| 9913 | Dry | Cationic | 5 | High | >85% | 0.75 | | |
| 7191 PLUS | Latex | Cationic | 5 | V. High | 26 | 1.03 | | |
| 8110 PULV | Dry | Cationic | 10 | High | >85% | 0.75 | NSF | |
| 9905 | Dry | Cationic | 10 | High | >85% | 0.75 | | |
| 71305 | Latex | Cationic | 10 | V. High | 35 | 1.02 | | |
| 7199 | Latex | Cationic | 10 | V. High | 26 | 1.02 | NSF | |
| 1T03 | Latex | Cationic | 10 | Medium | 35 | 1.04 | | |
| 9914 | Dry | Cationic | 15 | High | >85% | 0.80 | | |
| 9907 | Dry | Cationic | 20 | High | >85% | 0.80 | | |
| 7192 PLUS | Latex | Cationic | 20 | Medium | 35 | 1.03 | | |
| GR-206 | Latex | Cationic | 30 | High | 30 | 1.03 | | GRAS |
| 9908 | Dry | Cationic | 30 | High | >85% | 0.80 | | |
| 71303 | Latex | Cationic | 30 | V. High | 42 | 1.03 | | |
| 71302 | Latex | Cationic | 30 | V. High- | 42 | 1.03 | | |
| | | | | structured | | | | |
| 1T69 | Latex | Cationic | 35 | High | 35 | 1.03 | | |
| 7751 | Dispersion | Cationic | 35 | Medium | 20 | 1.19 | | |
| 7139 PLUS | Latex | Cationic | 40 | Medium- | 35 | 1.02 | | |
| | | | | High | | | | |
| GR-503 | Dry | Cationic | 45 | V. High | >85% | 0.85 | | GRAS |
| 9909 | Dry | Cationic | 50 | High | >85% | 0.80 | | |
| 71301 | Latex | Cationic | 50 | V. High | 42 | 1.04 | | |
| GR-201 | Latex | Cationic | 50 | V. High | 42 | 1.04 | | GRAS |
| 71300 | Latex | Cationic | 50 | V. High- | 42 | 1.02 | | |
| | | | | structured | | | | |
| 1404 | Latex | Cationic | 63 | High | 30 | 1.02 | | |
| GR-204 | Latex | Cationic | 65 | High | 30 | 1.02 | | GRAS |
| 71306 | Latex | Cationic | 65 | V. High- | 42 | 1.03 | | |
| | | | | structured | | | | |
| 71307 | Latex | Cationic | 65 | V. High | 42 | 1.02 | | |
| 9916 | Dry | Cationic | 70 | High | >85% | 0.80 | | |
| GR-505 | Dry | Cationic | 80 | High | >85% | 0.62 | | GRAS |
| 7181 | Latex | Nonionic | 0 | High | 26 | 1.04 | | |
| 8181 | Latex | Nonionic | 0 | High | 26 | 1.04 | NSF | W1 |
| 7182 | Latex | Anionic | 7 | High | 28 | 1.01 | | |
| 8182 | Latex | Anionic | 7 | High | 28 | 1.01 | NSF | W1 |
| 7769 | Latex | Anionic | 12 | High | 30 | 1.06 | | |
| 7766 PLUS | Latex | Anionic | 20 | Very V. | 30 | 1.03 | | |
| | | | | High | | | | |
| 7763 | Latex | Anionic | 30 | V. High | 30 | 1.04 | | |
| 8184 | Latex | Anionic | 30 | V. High | 30 | 1.04 | NSF | W1, W2 |
| 71325 | Latex | Anionic | 30 | V. High | 40 | 1.06 | NSF | |
| GR-105 | Latex | Anionic | 30 | V. High | 30 | 1.04 | | GRAS |
| GR-602 | Dry | Anionic | 30 | V. High | >85% | 0.80 | | GRAS |
| 7744 | Solution | Anionic | 30 | V. High | 2 | 1.02 | NSF | W1 |
| 7768 | Latex | Anionic | 30 | Very V. | 30 | 1.04 | NSF | |
| | | | | High | | | | |
| 1C34 | Latex | Anionic | 30 | Very V. | 30 | 1.08 | NSF | |
| | | | | High | | | | |
| 7767 | Latex | Anionic | 50 | V. High | 30 | 1.06 | | |
| 7878 | Latex | Anionic | 100 | High | 30 | 1.10 | | |

TABLE 5
A list of Nalco organic and blend coagulants tested and their properties.

| | | Organic | Inorganic | | | | | | |
|---|---|---|---|---|---|---|---|---|---|
| Product | Type | Component | Component | Chemistry | Actives | Form | S.G. | NSF | Other |
| LS | Organic | DADMAC | | High MW | 10% | Solution | 1.02 | NSF | |
| | | | | PolyDADMAC | | | | | |
| 8108 | Organic | DADMAC | | High MW | 18% | Solution | 1.04 | NSF | W1 |
| Plus | | | | PolyDADMAC | | | | | |
| 8102 | Organic | DADMAC | | Med. Low MW | 20% | Solution | 1.02 | NSF | W1 |
| Plus | | | | PolyDADMAC | | | | | |
| 8102 | Organic | DADMAC | | Med. Low MW | 20% | Solution | 1.10 | NSF | |
| | | | | PolyDADMAC- | | | | | |
| | | | | Salted | | | | | |
| 8103 | Organic | DADMAC | | Med. High MW | 20% | Solution | 1.02 | NSF | W1, |
| Plus | | | | PolyDADMAC | | | | | Kosher |
| 71259 | Organic | DADMAC | | Low MW | 30% | Solution | 1.09 | NSF | |
| | | | | PolyDADMAC | | | | | |
| 8799 | Organic | DADMAC | | Med. Low MW | 30% | Solution | 1.07 | | |
| LS | | | | PolyDADMAC | | | | | |
| 8799 | Organic | DADMAC | | Med. Low MW | 37% | Solution | 1.07 | NSF | W1, |
| Plus | | | | PolyDADMAC | | | | | Kosher |
| 7135 | Organic | EPI DMA | | Med. High MW | 50% | Solution | 1.14 | | |
| | | | | HMDA Crosslinked | | | | | |
| 8105 | Organic | EPI DMA | | Low MW Linear | 55% | Solution | 1.16 | NSF | W1, |
| | | | | Epi-DMA | | | | | Kosher |
| 8190 | Organic | DADMAC/AA | | Med MW | 20% | Solution | 1.04 | | |
| | | | | 90/10 Charge Ratio | | | | | |
| GR-410 | Blend | DADMAC | Aluminum | 65:35, Alum:GR-308 | — | Solution | 1.23 | | GRAS |
| GR-411 | Blend | DADMAC | Aluminum | 75:25, Alum:GR-308 | — | Solution | 1.28 | | GRAS |
| GR-400 | Blend | EPI DMA | Aluminum | 65:35, Alum:GR-305 | — | Solution | 1.26 | | GRAS |
| GR-401 | Blend | EPI DMA | Aluminum | 75:25, Alum:GR-305 | — | Solution | 1.29 | | GRAS |

TABLE 6
A list of Ashland cationic emulsion polymers tested and their properties.

| | | | | | | SOLUTION | | |
|---|---|---|---|---|---|---|---|---|
| | | | | | SOLUTION | VISCOCITY | | |
| PRAESTOL | | | | PRODUCT | VISCOSITY | 1.0% IN 10% | FREEZING | |
| POLYMER | CATIONIC | ACTIVE | DENSITY | VISCOSITY | 1% IN DIST. | NaCl-Brine ⁽²⁾ | POINT | EFFECTIVE |
| GRADE | CHARGE | CONTENT | (GR/ML) | (CP) | WATER⁽¹⁾ (CP) | (CP) | (□ □ □ ° C.) | pH RANGE |
| K105L | Low | 30% | 1.04 | <4000 | >5000 | >2000 | −15 | 1-10 |
| K110FL | Low | 35% | 1.03 | <4000 | >3000 | >1000 | −15 | 1-10 |
| K120L | Low-Medium | 40% | 1.03 | <4000 | >7000 | >500 | −15 | 1-10 |
| K226FLX | Medium | 29% | 1.03 | <4500 | >8000 | >400 | −15 | 1-10 |
| K111L | Medium | 40% | 1.03 | <4000 | >7000 | >500 | −15 | 1-10 |
| K122L | High | 43% | 1.04 | <4000 | >9000 | >300 | −15 | 1-10 |
| K128L | High | 43% | 1.04 | <4500 | >9000 | >900 | −15 | 1-10 |
| K132L | High | 35% | 1.01 | <5500 | >8000 | >300 | −15 | 1-10 |
| K133L | High | 44% | 1.05 | <4000 | >8000 | >150 | −15 | 1-13 |
| K136L | High | 44% | 1.05 | <4000 | >8000 | >300 | −15 | 1-13 |
| K290FL | Very High | 35% | 1.04 | <1800 | >8000 | >400 | −15 | 1-13 |
| K280FL | Very High | 36% | 1.04 | <1500 | >8000 | >200 | −15 | 1-13 |
| K290FLX | Very High | 44% | 1.04 | <2000 | >9000 | >500 | −15 | 1-13 |
| K144L | Very High | 46% | 1.04 | <4500 | >9000 | >200 | −15 | 1-13 |
| K148L | Very High | 46% | 1.04 | <5500 | >10000 | >600 | −15 | 1-13 |
| K275FLX | Very High | 46% | 1.04 | <5500 | >10000 | >400 | −15 | 1-13 |
| K260FL | Very High | 46% | 1.04 | <5500 | >10000 | >400 | −15 | 1-13 |
| K274FLX | Very High | 46% | 1.04 | <5500 | >10000 | >400 | −15 | 1-13 |
| K295FL | Very High | 40% | 1.04 | <2500 | >8000 | >350 | −15 | 1-13 |
| K279FLX • | Very High | 46% | 1.04 | <5500 | >10000 | >100 | −15 | 1-13 |

TABLE 7
A list of Ashland cationic granular polymers tested and their properties.

| | | | | SOLUTION | |
|---|---|---|---|---|---|
| | | | SOLUTION | VISCOSITY | |
| PRAESTOL | | BULK | VISCOSITY | 1% in 10% | |
| POLYMER | CATIONIC | DENSITY | % IN DIST. | NaCL-BRlNE ⁽²⁾ | EFFECTIVE |
| GRADE | CHARGE | (LBS/FT³) | WATER ⁽¹⁾ CP | CP | pH RANGE |
| 610BC | Low | 36 | >1000 | >400 | 1-13 |
| 321TR | Medium | 36 | >3000 | >300 | 1-13 |
| 611BC | Medium | 36 | >2500 | >400 | 1-13 |
| 851BC/TR | Medium | 36 | >3000 | <550 | 1-13 |
| 835BS | Medium | 38 | >5000 | >500 | 1-13 |
| 650BC/TR | High | 36 | >3500 | >250 | 1-13 |
| 852BC | High | 36 | >4000 | >400 | 1-13 |
| 644BC | Very High | 36 | >5000 | >200 | 1-13 |
| 853BC | Very High | 36 | >5500 | >350 | 1-13 |
| 855BS | Very High | 38 | >5000 | >500 | 1-13 |
| 655BS | Extremely High | 38 | >6000 | >500 | 1-13 |
| 857BS | Extremely High | 38 | >5000 | >370 | 1-13 |
| 658BS | Extremely High | 38 | >2500 | >500 | 1-13 |
| 858BS | Extremely High | 38 | >5000 | >500 | 1-13 |
| 859BS | Extremely High | 38 | >5000 | >350 | 1-13 |

TABLE 8
A list of Ashland cationic coagulant tested and their properties.

| PRAESTOL | Product | | | Approximate | |
|---|---|---|---|---|---|
| Product | Viscosity | Neat pH | Solids (%) | Molecular Weight | Type |
| 186K | 60-180 | 5-8 | 19.0-21.0 | 150,000 | polyDADMAC |
| 186KH | 200-500 | 5-8 | 19.0-21.0 | 250,000 | polyDADMAC |
| 187K | 1000-3000 | 5-8 | 39.0-41.0 | 150,000 | polyDADMAC |
| 187KH | 8000-13000 | 5-8 | 39.0-41.0 | 250,000 | polyDADMAC |
| 188K | 80-200 | 6-8 | 48.5-51.0 | 25,000 | polyamine |
| 189K | 5000-9000 | 4-7 | 48.5-51.0 | 250,000 | polyamine |
| 193K | 600-1000 | 5-7 | 48.0-51.0 | 75,000 | polyamine |
| 40176 | 1000-3000 | 5-8 | 19.0-21.0 | 400,000 | polyDADMAC |
| CM-175 | 1700 minimum | 3-5 | 24.0-26.5 | 500,000 | PAA (anionic) |

TABLE 9
A list of Ashland polymers tested and their properties.

| | Product | | | | |
|---|---|---|---|---|---|
| PRAESTOL | Viscosity | 10% pH | Solids | Specific | |
| Product | (cps) | Viscosity | (%) | Gravity | Composition |
| K2000 | “water” | ≅2.5 | ≅40 | 1.28-1.29 | AC |
| K2001 | “water” | ≅4.5 | ≅50 | 1.33-1.35 | ACH |
| K2002 | “water” | 2-3 | ≅40 | 1.25-1.28 | AC + |
| | | | | | solution |
| | | | | | poly |
| K2002A | “water” | 2.5-3.5 | ≅40 | 1.26-1.28 | AC + |
| | | | | | ACH + |
| | | | | | solution |
| | | | | | poly |
| K2003 | “water” | ≅4 | ≅20 | 1.19 | PAC |
| | | | | minimum | |
| K2004 | “water” | 4-5 | ≅40 | 1.31-1.34 | ACH + |
| | | | | | solution |
| | | | | | poly |
| K2005A | “water” | 4-5 | ≅45 | 1.24-1.27 | ACH + |
| | | | | | solution |
| | | | | | poly |
| K2006 | 1300-1900 | 3-5 | ≅30 | 1.10-1.13 | PAC + |
| | | | | | solution |
| | | | | | poly (2) |
| K2007 | “water” | 1-2 neat | ≅30 | 1.24-1.28 | PAC + Al |
| | | | | | phosphate + |
| | | | | | solution |
| | | | | | poly |

TABLE 10
A list of Ashland emulsion polymers tested and their properties.

| | | | | | | SOLUTION | | |
|---|---|---|---|---|---|---|---|---|
| | | | | | SOLUTION | VISCOCITY | | |
| PRAESTOL | | | | PRODUCT | VISCOSITY | 0.5% IN 10% | FREEZING | |
| POLYMER | ANIONIC | ACTIVE | DENSITY | VISCOSITY | 0.5% IN DIST. | NaCl-Brine ⁽²⁾ | POINT | EFFECTIVE |
| GRADE | CHARGE | CONTENT | (GR/ML) | (CP) | WATER⁽¹⁾ | (CP) | (° C.) | pH RANGE |
| N3100LTR | Nonionic | 27% | 1.03 | <3000 | >300 | >300 | −15 | 0-13 |
| A3010LTR | Low | 30% | 1.04 | <1700 | >6500 | >400 | −15 | 1-13 |
| A3025L | Medium | 32% | 1.07 | <4500 | >5000 | >175 | −15 | 5-13 |
| A3030L | Medium | 36% | 1.09 | <4000 | >4000 | >160 | −15 | 5-13 |
| A3040L | Medium | 31% | 1.07 | <3100 | >8000 | >400 | −15 | 6-13 |
| A3040LTR | Medium | 31% | 1.07 | <3100 | >7300 | >400 | −15 | 6-13 |
| A4040L | Medium | 40% | 1.10 | <3800 | >6000 | >175 | −15 | 6-13 |
| A3050L | High | 40% | 1.12 | <4000 | >10000 | >400 | −15 | 6-13 |
| A3095L | High | 33% | 1.10 | <2000 | >5000 | >40 | −15 | 7-14 |

TABLE 11
A list of Ashland granular polymers tested and their properties.

| | | | | SOLUTION | |
|---|---|---|---|---|---|
| | | | SOLUTION | VISCOSITY | |
| PRAESTOL | | BULK | VISCOSITY | 1% in 10% | |
| POLYMER | ANIONIC | DENSITY | % IN DIST. | NaCL-BRINE ⁽²⁾ | EFFECTIVE |
| GRADE | CHARGE | (LBS/FT³) | WATER ⁽¹⁾ CP | CP | pH RANGE |
| 2500/2500T | Nonionic | 41 | >200 | >140/>60 | 0-13 |
| 2510 | Low | 43 | >300 | >140 | 5-13 |
| 2515/2515TR | Low | 42 | >2000 | >180 | 5-12 |
| 2520 | Low | 43 | >3500 | >180 | 6-13 |
| 2525 | Low | 43 | 3000 | 200 | 5-13 |
| 2530/2530T | Medium | 43 | >5000 | >200 | 6-13 |
| 2540/2540T | Medium | 44 | >4500 | >200 | 6-13 |
| 2640 | Medium | 43 | >4000 | >200 | 6-13 |

### Polymer Flocculation Efficiency Testing

The following procedure is used to assess polymer effectiveness. Forty (40) mL SE0087 culture in MASM 16 ppt is gathered in sets of seven (7) so that a proper dose response can be applied. A maximum allowable use of polymer is set at 15-20 mg polymer/L of algae. Volumetrically, polymer dosing is increased at an increment of 40 uL stock solution. Every 40 uL of 0.25% polymer stock solution leads to a working concentration increase of 2.5 mg/L. To each of the 40 mL samples of SE0087 culture, 0, 40, 80, 120, 160, 200, or 400 uL polymer stock solution is added. This corresponds to a polymer dosing of 2.5, 5, 7.5, 10, 12.5 and 25 mg/L in the culture. A dose response curve is then made for each polymer that is effective and selected for further screening. Control samples are also used to determine the response to polymer. An indirect method is used for efficiency calculation (see above) due to low culture density. Upon the addition of polymer, samples are rapidly mixed for 10 seconds, and then placed on a shaker table at 120 RPM for 10 minutes. Cultures are then allowed to settle for 15 minutes. The resulting subnatant is sampled for efficiency calculations.

Polymers from Monolyte

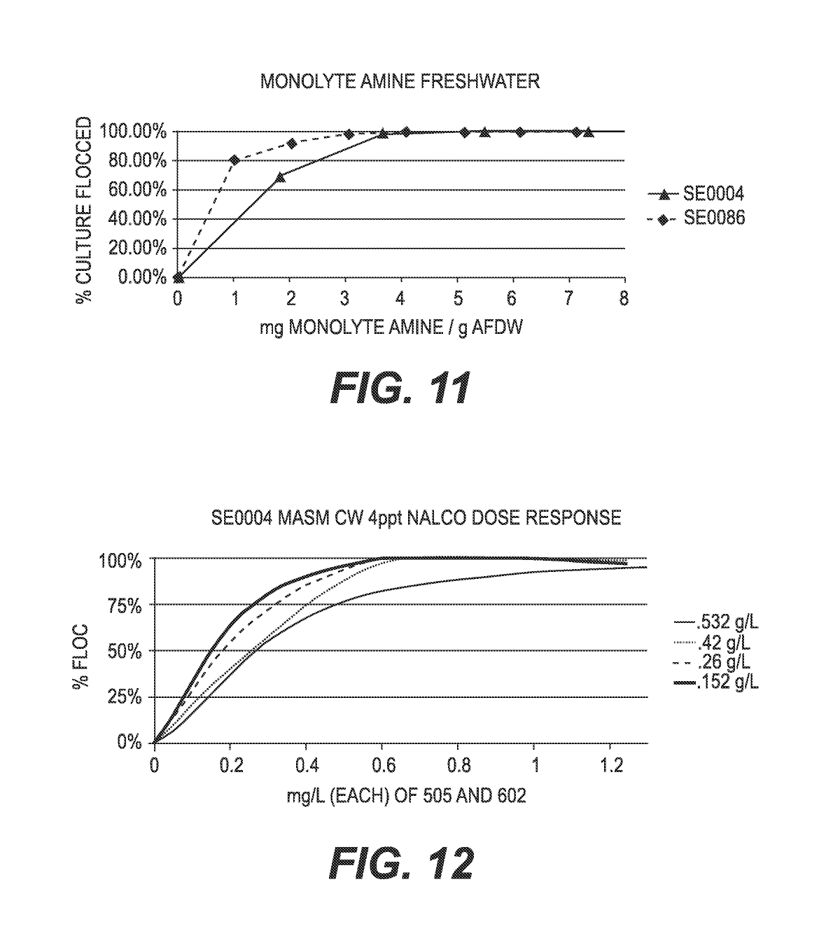

Polymers from Monolyte are tested in IL jar testers with algae strains SE0004 and SE0086 grown in G-media. SE0004 and SE0086 are low salt strains and show excellent response to Monolyte polymer CE-3990 in G-Media. Very low doses of CE-3990 are required to flocculate the algae. Flocculation efficiency is calculated using the indirect method. Floc sizes are large and fluffy (Table 12). FIG. 11 shows a CE-3990 dose response standardized to 1 g/L of AFDW algae based on the initial concentration of culture. The SE0004 and SE0086 culture concentrations are 0.544 g/L and 0.974 g/L, respectively. Polymer CE-3990 is further tested in the field with SE0004 and SE0086 cultures grown in MASM media. In these tests, both SE0004 and SE0086 have difficulty to flocculate as the amount of chloride in the MASM media prevents polymer from unfolding properly.

TABLE 12
Flocculation of strain SE0004 in G-
Media with Monolyte polymer CE-3990

| CE-3990 | SE0004 Supernatant | |
|---|---|---|
| (ppm, mg/L) | (g/L) G-Media | % Flocced |
| 0 | 2.284 | 0% |
| 5 | 0.3 | 84.83% |
| 7 | 0.13 | 94.31% |
| 9 | 0.048 | 97.9% |
| 11 | 0.004 | >99% |
| 13 | 0 | >99% |
| 15 | 0 | >99% |
| 17 | 0 | <1% |
| 19 | 0 | <1% |

Polymers from Nalco

A broad screening of polymer solutions from Nalco is performed at Nalco's Center of Innovative Research in South Carolina with sample strains SE0087 and SE0004. The initial efforts focus on Generally Recognized As Safe (GRAS) certified polymers. Testing format includes single cationic or anionic polymers, combinations of anionic and cationic polymers, and individual cationic coagulants.

Nalco Polymer Screening with Strain SE0004

SE0004 samples are tested at its initial pH of 8.5. Two different approaches are used to screen: a flocculant-only program, and a coagulant-flocculant program. GRAS products and a non-GRAS coagulant are tested.

Flocculant only programs are tested using a procedure of flocculant addition followed by a 1-minute fast mix (250 rpm), a 3-minute slow mix (50 rpm), and a 30-minute settling time. Cationic flocculants are tested. A single cationic flocculant at 10 ppm achieves sufficient separation.

All of the GRAS cationic polymers, both latex and dry, are tested at 10, 20, 30, and 40 ppm. Turbidity is measured after treatment. All of the cationic polymers are equally effective in terms of turbidity reduction. The latex polymers perform better than dry polymers in achieving larger flocs. Among all polymers tested in the flocculant only program, latex polymer GR-206, which is the lowest mole charge flocculant in the tested group, and dry polymer GR-503, which also has lower mole charge, both are effective (Table 13 and Table 14). Anionic flocculants alone do not work effectively in these tests.

TABLE 13
Dose response of polymer GR-206 on strain SE0004.

| Polymer dosage (ppm) | 10 | 15 | 20 | 25 |
|---|---|---|---|---|
| Supernatant turbidity | 10.8 NTU | 15.0 NTU | 19.2 NTU | 21.2 NTU |

TABLE 14
Flocculation effectiveness of Nalco
coagulants program for strain SE0004.

Supernatant turbidity per dosage (NTU)

| Coagulant | 10 ppm | 20 ppm | 30 ppm | 40 ppm |
|---|---|---|---|---|
| GR-206 | 10.8 | 15.0 | 19.2 | 21.2 |
| GR-201 | 13.9 | 15.9 | 21.6 | 25.1 |
| GR-204 | 9.72 | 15.1 | 20.2 | 23.1 |
| GR-503 | 12.8 | 16.8 | 18.6 | 19.2 |
| GR-505 | 12.0 | 14.5 | 16.2 | 19.2 |

### Coagulant-Flocculant Combination Programs

GRAS polymers GR-401, GR-410, and GR-411, and NSF-approved polymer 8187 are tested in a coagulant-flocculant combination program. They are all tested at 100 ppm initially. Testing is performed with a procedure of coagulant addition followed by a 1-minute fast mix (250 rpm) during which flocculant is added at the 30 seconds mark, a 3-minute slow mix (50 rpm), and a 30-minute settling time. The supernatant is measured for turbidity.

In this coagulant-flocculant combination program, anionic flocculants are effective when combined with coagulants. Cationic polymers are not as effective as anionic ones. Among all coagulants tested in this GR-602 combination program, GR-410 is identified as the most effective coagulant, achieving the lowest turbidity, while polymer 8187 is the least effective (Table 15).

GR-410 is then tested with three available anionic flocculants: latex GR-105 and 7768 (NSF), and dry GR-602. Among them, GR-602 is the most effective at the lowest dosage, forming consistent flocs at all dosages (10, 20, and 30 ppm) (Table 16).

GR-410 and GR-602 are also tested in various combinations. No advantage is observed by adding flocculant before GR-410. Adding flocculant GR-602 first leads to increased turbidity. In summary, coagulant GR-410 is effective with an anionic flocculant. Depending upon desired turbidity, GR-410 can be used at 10 ppm in combination with 2 ppm GR-602 (Table 17 and Table 18).

TABLE 15
Flocculation effectiveness of a combination of a Nalco
anionic flocculant and a coagulant for strain SE0004.

| Coagulant, 100 ppm | GR-602 | Turbidity (NTU) |
|---|---|---|
| GR-401 | 20 ppm | 15.2 |
| GR-410 | | 9.51 |
| GR-411 | | 11.5 |
| 8187 | | 26.2 |

TABLE 16
Flocculation effectiveness of a combination of a Nalco anionic
flocculant and a cationic coagulant (GR-410) for strain SE0004.

| Flocculant (ppm) | GR-410 (ppm) | Turbidity (NTU) | Floc Size |
|---|---|---|---|
| GR-602 | 20 | | |
| 10 | | 8.09 | Large |
| 20 | | 10.5 | Large |
| 30 | | 11.9 | Large |
| GR-105 | 20 | | |
| 10 | | 14.2 | Small |
| 20 | | 16.2 | Large |
| 30 | | 14.5 | Large |
| 7768 | 20 | | |
| 10 | | 13.5 | Small |
| 20 | | 16.9 | Medium |
| 30 | | 21.6 | Large |

TABLE 17
GR-410 and GR-602 dose response on strain SE0004.

| | Polymer dosage (ppm) (GR-410 + GR-602) | | |
|---|---|---|---|
| | 10 + 2 | 50 + 5 | 100 + 10 |
| Supernatant turbidity | 15.0 NTU | 19.2 NTU | 21.2 NTU |

TABLE 18
Dosing effects of a GR-410 and GR-
602 combination for strain SE0004.

| R-410 (ppm) | GR-602 (ppm) | Turbidity (NTU) |
|---|---|---|
| 10 | 2 | 19.2 |
| 10 | 10 | 24.1 |
| 50 | 5 | 12.9 |
| 50 | 10 | 16.1 |
| 100 | 10 | 9.75 |

### Dosage Dependence on Culture Density

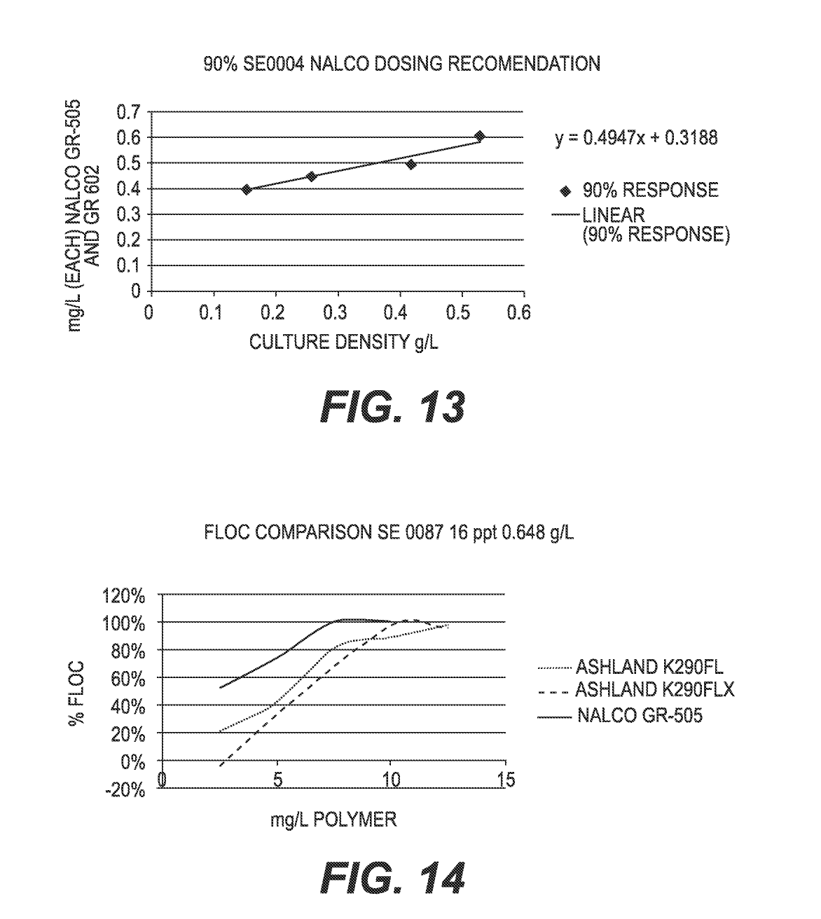

When using cationic polymers and anionic polymers together, a clear dosage dependence on culture density is observed. The more dense the culture is, the more polymer is required. Dose response curves for a polymer combination of cationic GR-505 and anionic GR-602 are generated for an optimal dosing of 90% flocculation efficiency (FIG. 12 and FIG. 13). The two-component polymer combination flocculates SE0004 efficiently.
Nalco Polymer Screening with Strain SE0087

SE0087 samples are tested at its initial pH of 8.5. SE0087 samples are too low in solids for a traditional dewatering process. Two different approaches of polymer-assisted dewatering are set forth: a flocculant-only program in addition to a coagulant-only program. Both GRAS and NSF products are tested, though GRAS products are more effective.

### Coagulant-Only Programs

In coagulant-only programs, GRAS coagulants GR-401, GR-410 and GR-411 are tested at 100, 200, and 400 ppm each. GR-401 has limited effectiveness. GR-410 achieves slightly lower supernatant turbidities than GR-411. GR-410 is effective at 200 ppm, with the lowest turbidity of 3.42 NTU (Table 19). For a coagulant, overnight settling is beneficial for the solids to settle to the bottom of the jar in order to obtain an accurate turbidity measurement.

TABLE 19
Nalco coagulant only program for strain SE0087.

Turbidity (NTU)

| Dosage (ppm) | GR-410 | GR-411 |
|---|---|---|
| 100 | 7.26 | 10.2 |
| 200 | 3.42 | 3.99 |
| 400 | 4.83 | 4.94 |

### Flocculant-Only Programs

Both dry and latex GRAS flocculants are tested in flocculant-only programs. Anionic flocculants alone are also not effective. Combinations ofa cationic polymer and an anionic polymer are tested. Effects of polymer adding order are tested. A cationic/anionic dual component system requires both types of flocculants in order to be effective. Adding anionic first, followed by cationic is more effective in the system.

Both latex anionic polymer and dry anionic polymer are tested in flocculant-only programs. A dry GRAS anionic polymer (GR-602) is effective. A latex GRAS anionic polymer (GR-105) is less effective, and an NSF latex polymer (7768) is adapted for a latex program.

A determination is then made regarding which form of Nalco cationic polymers has the best attributes. Two cationic GRAS latex polymers and two dry polymers from Nalco are tested. For both types of flocculants, the higher mole charge product is more effective. An effective latex cationic flocculant is GR-204, and an effective dry cationic flocculant is GR-505.

Based on the above determination, two cationic/anionic dual component systems are set up: a latex system with anionic 7768 and cationic GR-204, and a dry system with anionic GR-602 and cationic GR-505. For both systems, an effective ratio between anionic and cationic products is tested. It is determined that the products are effective when used in a 1:1 ratio.

A latex set of 7768 and GR-204 is dosed at 10+10, 15+15, 20+20, and 25+25 ppm. Increased polymer dosing leads to increased turbidity. The lowest turbidity is achieved with 10 ppm of each polymer, but the most effective solid formation is observed with 20 ppm of each polymer (Table 20 and Table 21). The supernatant is viscous due to the amount of polymer used, but the solids are not particularly sticky.

A dry set of GR-602 and GR-505 is dosed at 10+10, 15+15, 20+20, and 25+25 ppm, as well as 30+30 and 35+35 ppm. In this case, the lowest turbidity and most effective solid yields are produced with 20 ppm of each polymer (Table 22 and Table 23). Dry polymers have lower turbidities than the latex program overall. Dry polymers produce a more viscous supernatant and stickier solids compared to latex polymers.

TABLE 20
Nalco latex flocculant program for strain SE0087.
Latex Flocculant Program

| 7768 (ppm) | GR-204 (ppm) | Turbidity (NTU) |
|---|---|---|
| 10 | 10 | 40.6 |
| 15 | 15 | 56.6 |
| 20 | 20 | 103 |
| 25 | 25 | 122 |

TABLE 21
Dose response of Nalco's 7768 + GR-204 latex program.

| | Polymer dosage (ppm) (7768 + GR-204) | | | |
|---|---|---|---|---|
| | 10 + 10 | 15 + 15 | 20 + 20 | 25 + 25 |
| Supernatant turbidity | 40.6 NTU | 56.6 NTU | 103 NTU | 122 NTU |

TABLE 22
Dose response of Nalco's GR-505
and GR-602 dry flocculant program.

| | Polymer dosage (ppm) (GR-505 + GR-602) | | | |
|---|---|---|---|---|
| | 10 + 10 | 15 + 15 | 20 + 20 | 25 + 25 |
| Supernatant turbidity | 38.5 NTU | 37.1 NTU | 23.2 NTU | 31.6 NTU |

TABLE 23
Nalco's dry flocculant program for strain SE0087.
Dry Flocculant Program

| GR-602 (ppm) | GR-505 (ppm) | Turbidity (NTU) |
|---|---|---|
| 10 | 10 | 38.5 |
| 15 | 15 | 37.1 |
| 20 | 20 | 23.2 |
| 25 | 25 | 31.6 |
| 30 | 30 | 37.2 |
| 35 | 35 | 41.2 |

Coagulants are fed as 10% stock solutions. Latex flocculants are fed as 0.5% (w/w) stock solutions. Dry flocculants are fed as 0.25% (w/w) stock solutions.

Further lab tests with Nalco polymers are performed to improve the flocculation efficiency and reduce polymer costs. Dry Nalco polymers prove to be the most effective and easiest to use in the field for accurate dosing.

### Comparison Between Nalco GR-505 and Ashland Emulsion Polymers

Nalco dry cationic polymer GR-505 is tested against selected top-performing Ashland emulsion polymers to compare their performance. GR-505 is effective due to their 100% active content (FIG. 14). Emulsion based polymers typically have 30-40% active content so a greater quantity of material is needed to neutralize the charge of a colloid system.

### Comparison Between Nalco Non-GRAS Polymers and Ashland Polymer 859BS

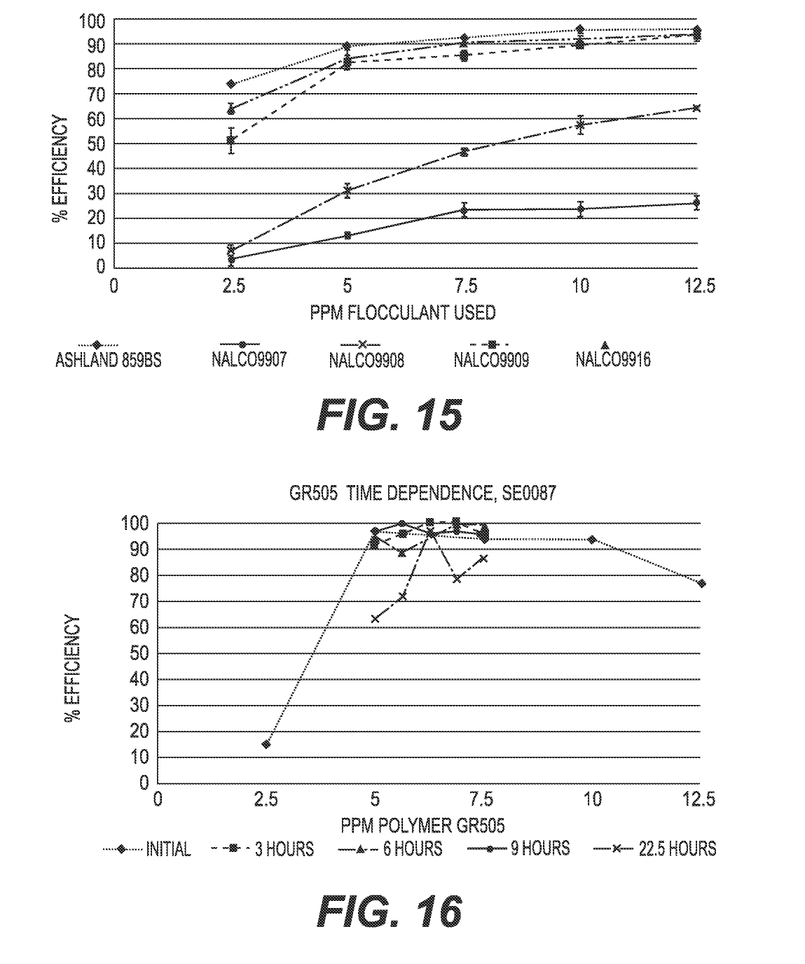

A number of non-GRAS polymers from Nalco are also tested. These non-GRAS polymers are similarly effective compared to Ashland polymer 859BS (FIG. 15). Further, GR-505 is similarly effective compared to 859BS, Nalco 9916, and 9909 (see FIG. 14 and FIG. 15).

### Further Evaluation of Nalco Dry Cationic Polymer GR-505

Performance of Nalco dry cationic polymer GR-505 is further evaluated in a number of aspects. First, the degradation of GR-505 stock solutions and the resulting decreased performance are examined. In general, polymers begin to hydrolyze when a stock solution is prepared in water. GR-505 stock solution of 0.25% is tested over several time points up to 22 hours following stock preparation. A slight degradation and decreased performance is observed in a 9 hour old GR-505 stock solution (FIG. 16).

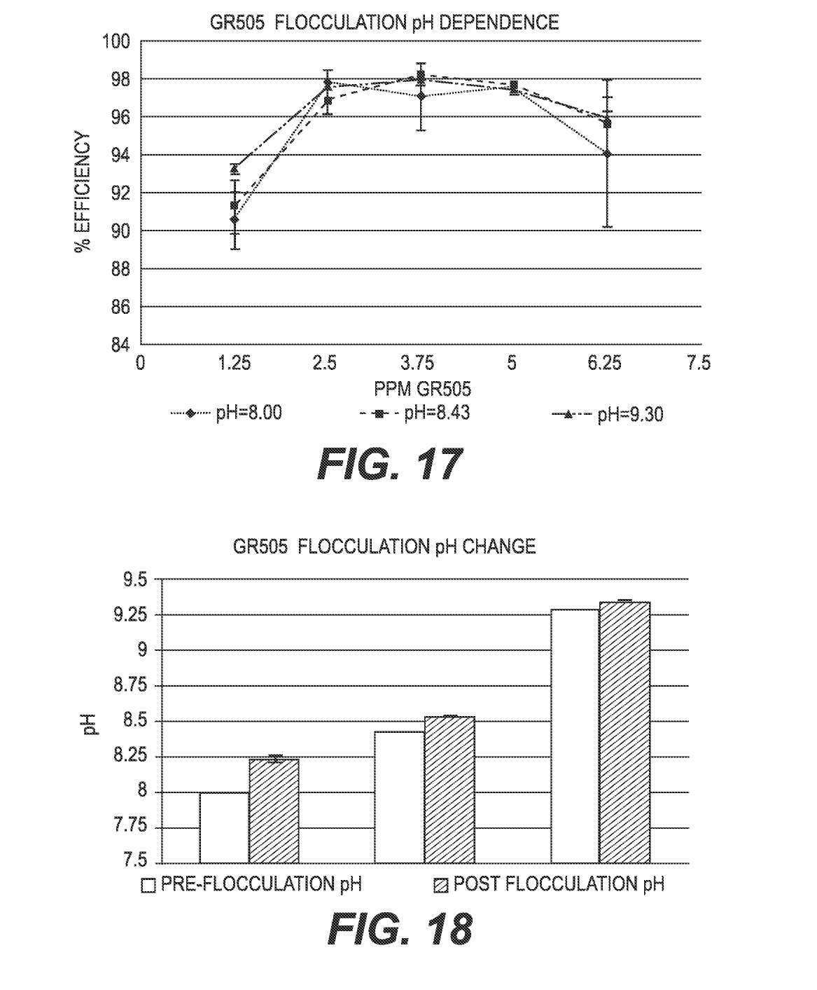

Next, the operational pH range of GR-505 is tested. The operational pH range of GR-505 is found to be within the operating range of algae cultures and shows no decreased performance between pH 8 to 9.3 (FIG. 17). Further, pH of the media upon flocculation by GR-505 is monitored. When using GR-505 for flocculation, the resulting media have a slightly higher pH (FIG. 18).

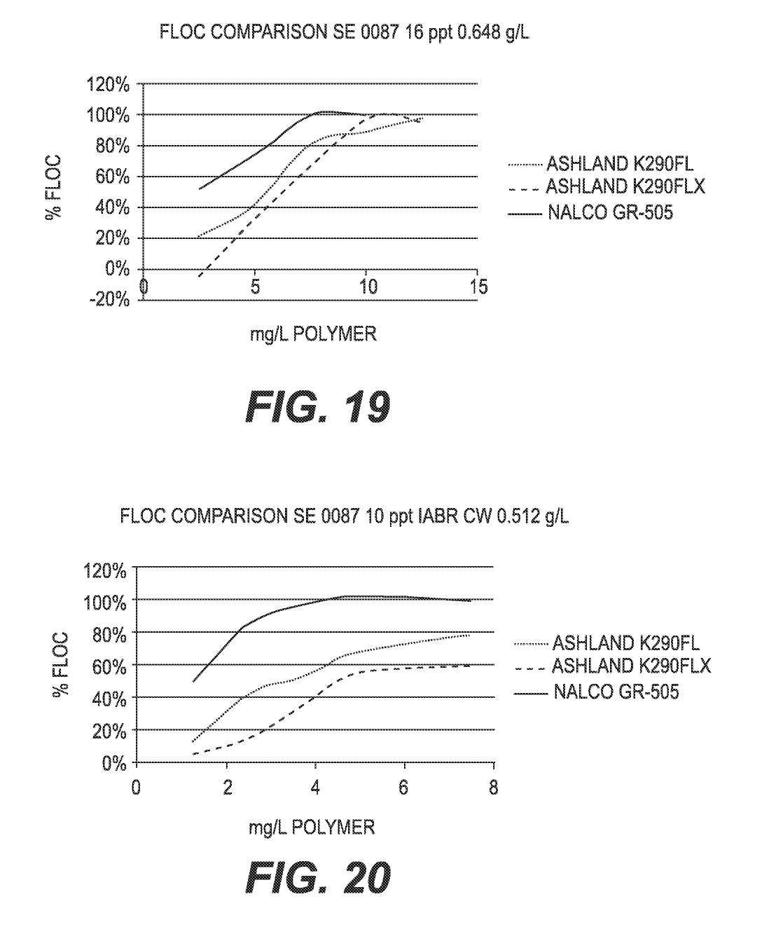

Lastly, flocculation effectiveness of GR-505 is also tested in various media with different levels of TDS. Polymers from Nalco and emulsions from Ashland are both effective in MASM 16 ppt and in IABR 6 ppt media (FIG. 19 and FIG. 20). One of the main differences between MASM and IABR media is that the IABR media uses the acid forms of nutrients which eliminates some of the Total Dissolved Solids (TDS) and improves flocculation efficiency.

Polymers from Ashland

A collection of different types of polymers from Ashland are screened for their effectiveness to flocculate algae strain SE0087 from MASM 16 ppt. Testing results are described in sections below.

### Ashland Coagulants

Organic coagulants and blended organic coagulants from Ashland are both tested (Table 24). These coagulants typically have a very high cationic charge and a very low molecular weight, and are designed to disrupt the charge of a system to allow polymer flocculants to be more effective.

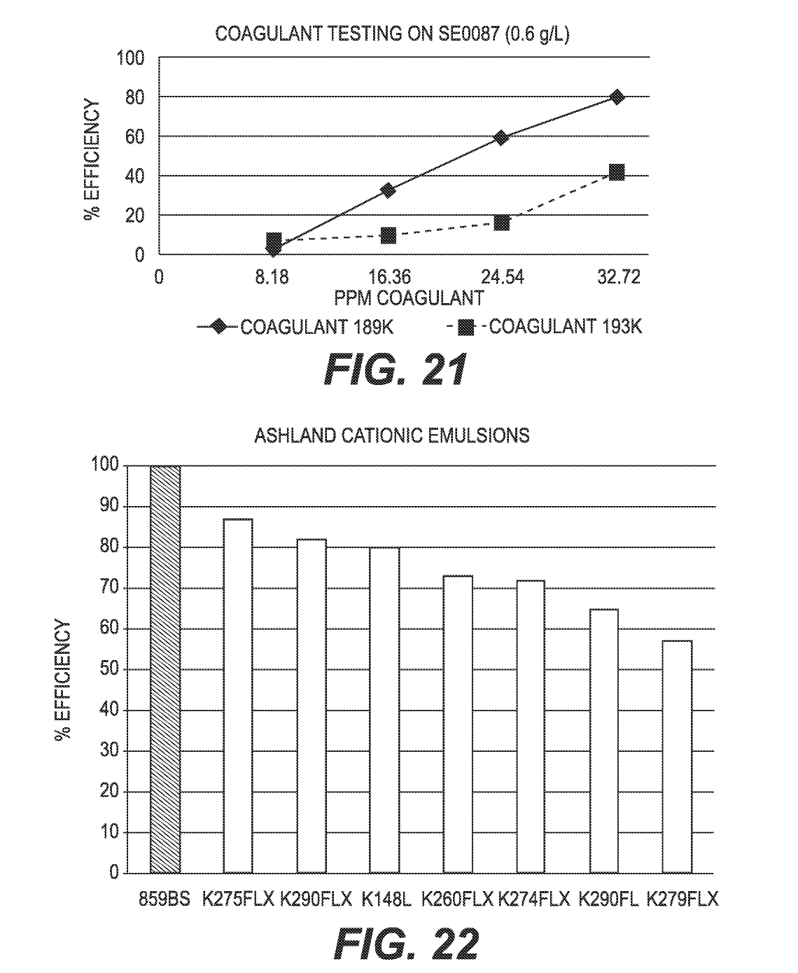

Coagulants that flocculate SE0087 from MASM effectively are 189K and 193K (FIG. 21). Though they are required in fairly high doses, their success nevertheless demonstrates that coagulants can be a viable option for algal dewatering if the cost can be reduced.

TABLE 24
Ashland Coagulants tested on SE0087 in MASM media.
Ashland Coagulants

| | |
|---|---|
| 186K | polyDADMAC |
| 186KH | polyDADMAC |
| 187K | polyDADMAC |
| 187KH | polyDADMAC |
| 188K | polyamine |
| 189K | polyamine |
| 193K | polyamine |

### Ashland Emulsion Polymers

A number of cationic emulsion polymers do not perform effective in flocculating algae and are not further screened (Table 25). Candidate polymers from the screening are then tested side by side to compare their effectiveness (FIG. 22). Ashland dry polymer 859BS is used as a benchmark.

For emulsion polymers from Ashland, a candidate is K275 FLX. This is a highly charged cationic polymer that contains 46% active material. Other emulsions that are effective have high charge, high molecular weight, and high active content. None of the Ashland emulsion polymers perform as effectively as 859BS. Further, the tested Ashland emulsion polymers use a working concentration of roughly 12.5 mg/L for effective flocculation. This concentration is roughly twice the amount required for 859BS.

TABLE 25
A list of Ashland Cationic Emulsion polymers
not effective in flocculating strain SE0087.

K110FL
K105FL
K111L
K120L
K122L
K128L
K132L
K133L
K136L
K144L

### Further Evaluation of Ashland Polymer K279FLX

Ashland polymer K279FLX is tested to explore the limitations of cationic polymers and what the major driving factors are to improve its effectiveness.

### Mixing Effects

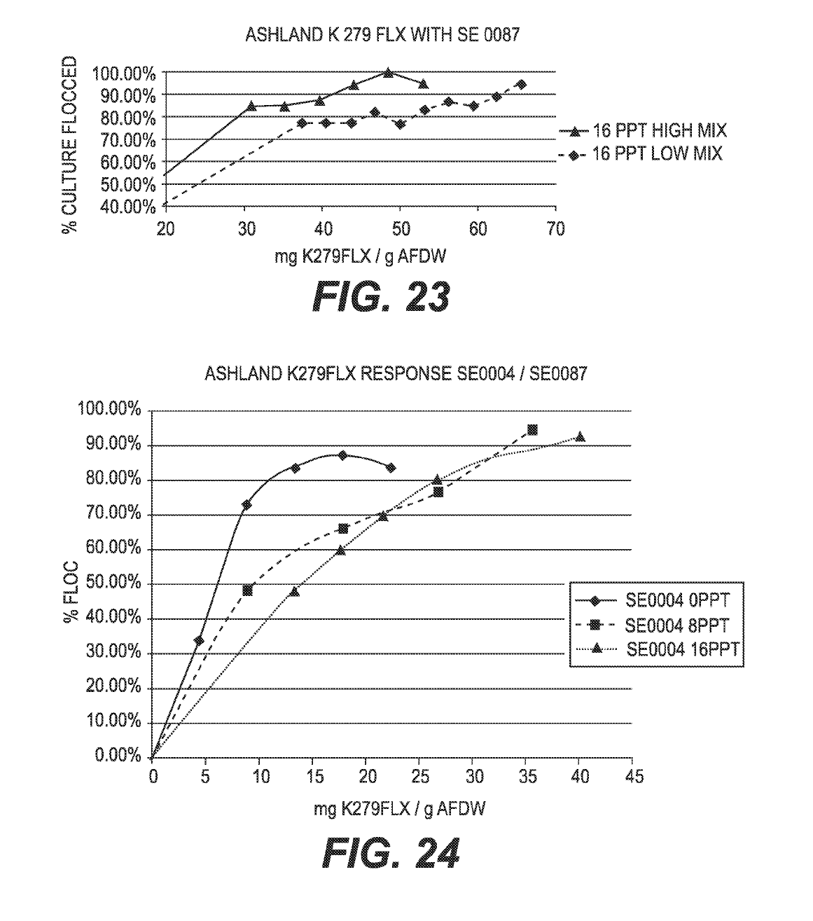

Impacts on flocculation efficiency from polymer mixing is evaluated. With polymer K279FLX, a culture that is treated with high initial mixing of 15 sec of 300RPM in a jar tester followed by 10 minutes of 50RPM mixing flocculates better compared to a culture that is only mixed at 50RPM for 10 minutes (FIG. 23). Therefore, the mixing and addition of a polymer can be important to the flocculation efficiency. A rapid mix is beneficial to get the 0.25% stock solution in the culture and then the slow mixing is beneficial to improve the kinetic reactions that are occurring. If mixing is too fast then negative impacts can occur with the shearing of the polymers.

### Salt Dependence

Cationic polymers' effectiveness can be salt dependent because salts impact the unfolding of polymer in solution. Cultures of SE0004 and SE0087 are grown up in G-Media and MASM, respectively. To prepare algae culture with different TDS levels, salts are added to the SE0004 culture while the SE0087 culture is spun down and resuspended in desired salt concentrations.

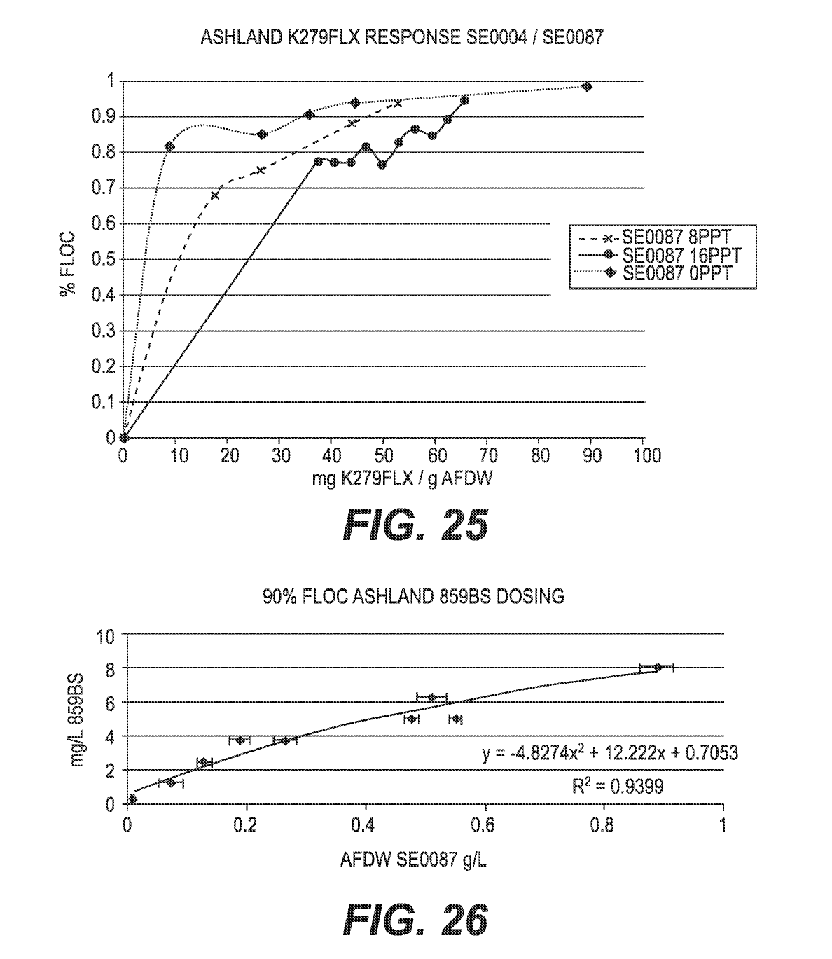

A linear relationship between the TDS of a solution and the effectiveness of given cationic polymers is observed (FIG. 24 and FIG. 25). Prevention of full unfolding of a polymer due to the ionic strength of a solution decreases the efficiency of the polymer. Higher molecular weight polymers have the ability to unfold less than lower molecular weight polymers while still maintaining a large surface area for binding particles in solution.

### Ashland Dry Polymer 859BS

A collection of dry polymers from Ashland are tested for SE0087 flocculation from MASM 16 ppt. Most are not high performers (Table 26). A high performing polymer from Ashland that flocculates SE0087 efficiently is cationic dry polymer 859BS.

Dry polymer 859BS is subjected to additional testing to explore its limitations and the major driving factors for improving its effectiveness. Polymer 859BS provides the lowest dosing required among all polymers tested. A dose response curve based on culture density in a 16 ppt NaCL MASM culture shows that a polymer dosing of less than 8 mg/L effectively flocculates an SE0087 culture at 0.9 g/L (FIG. 26). Polymer 859BS is implemented in an improved DAF process in Columbus, N. Mex.

TABLE 26
A list of Ashland cationic granular polymers showing no
response in flocculating SE0087 in MASM 16 ppt media.

610BC
611BC
650BC
655BC
655BS
658BS
644BC
822BS
835BS
851BC
852BC
853BC
855BS
857BS
858BS

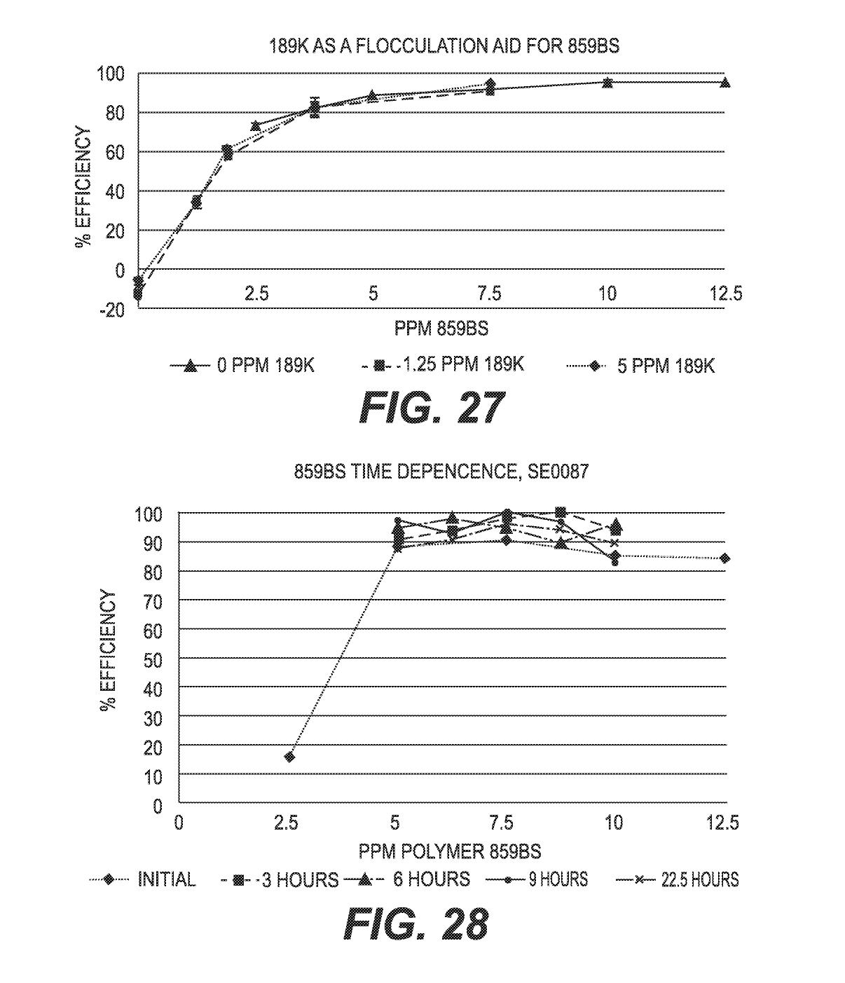

Polymer 859BS is tested in combination with Ashland coagulant 189K. This coagulant-flocculant combination does not improve the efficiency of 859BS and requires the same amount of polymer regardless of the level of coagulant (FIG. 27).

The degradation of 859BS stock solutions and the resulting decreased performance are examined. In general, polymers begin to hydrolyze upon stock solution is prepared in water. Stock solutions of 859BS at a concentration of 0.25% are tested over several time points up to 22 hours following stock preparation. No decrease in polymer performance is observed when a stock solution was allowed to sit for up to 22.5 hours (FIG. 28). This is an improvement over Nalco cationic dry polymer GR-505, which shows degradation over time due to hydrolysis. Field conditions most likely decrease polymer activity as polymers may be exposed to sunlight for extended periods of time.

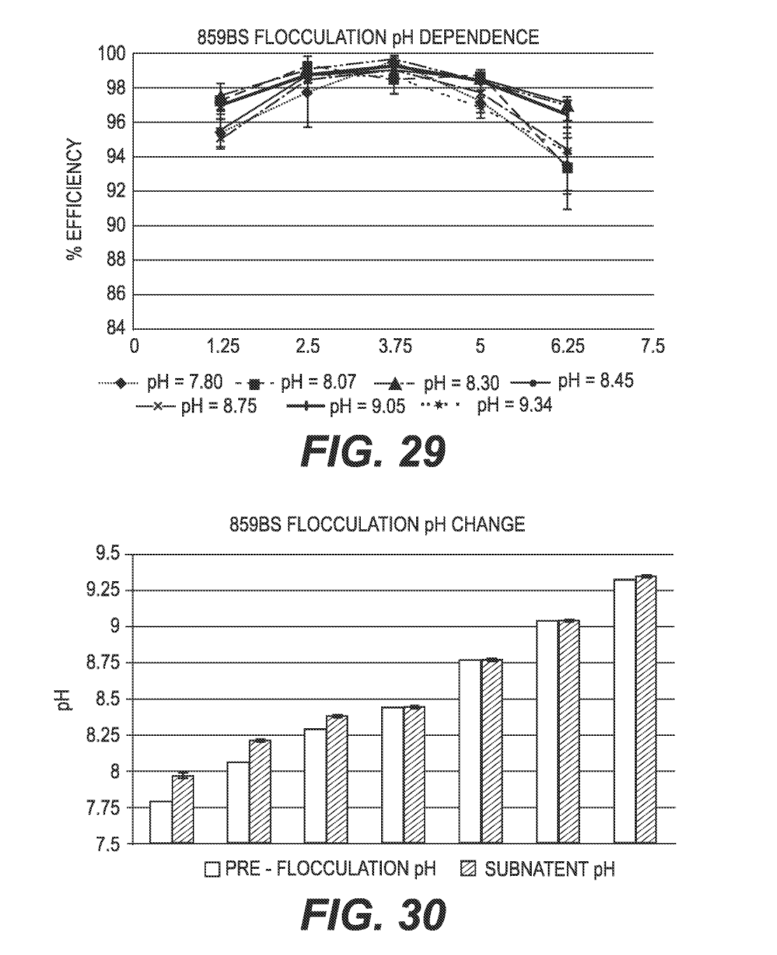

Next, the operational pH range of 859BS is examined as a polymer that works in diverse and flexible culture conditions is sought. Operational pH range of 859BS is found to be within the preferred operating range of algae cultures (pH between 8 and 10). It also shows no performance decrease between pH 7.8 and 9.3 (FIG. 29). Furthermore, the pH of MASM media only changes upon flocculation with 859BS when the initial culture pH was below 8.5 (FIG. 30). This pH impact to algae culture needs to be closely monitored and taken into account when media are recycled or applied with bleach.

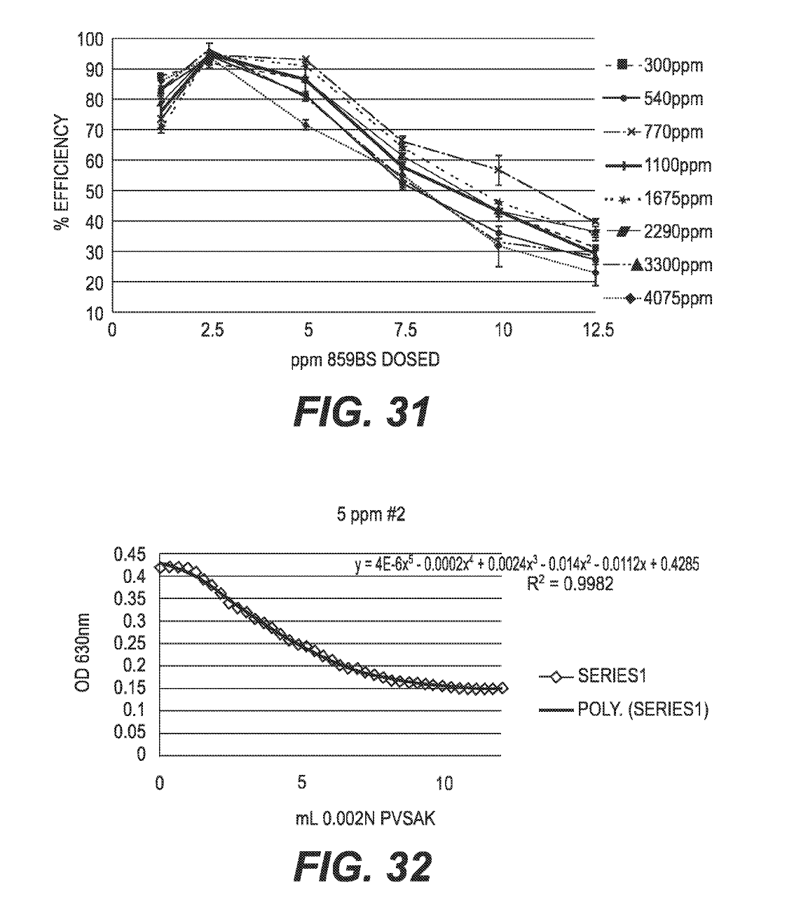

Lastly, flocculation efficiency of 859BS is also tested in media with various alkalinities. It is observed that alkalinity has little to no effect on 859BS (FIG. 31). Tests shown in FIG. 31 also illustrates that increased loading of polymer may not be desired for optimal flocculation. As a system is over polymerized, polymers begin to bind themselves and other polymer particles in the same solution which reduces the effective surface binding area and the colloidal distribution in the media.

### Example 6

### Polymer Quantification

The amount of polymer required for dewatering algae is preferably kept to a minimum dosage. One way to optimize polymer dosage and loading is to minimize the percentage of polymers that are not bound to algae and remain in the supernatant upon flocculation. As supernatant media are to be recycled to support future algae growth, a significant amount of polymer in the recycled media may have detrimental effects. There is a potential for flocculation to happen in pond when recycled media contain substantial amount of polymer, which can have dramatic negative impacts to the growth of algae. Further, the determination of the required minimal polymer dosage is also critical to cost evaluation as it is highly desired to have 100% of polymers directed to floes and none going to waste. As such, polymers that are not bound to algae and remain in the aqueous solution need to be identified and quantified.

### Development of a Colorimetric Test and Calibration Curve

A colorimetric test is developed to measure the amount of cationic polymers in media using an anionic acid and a colorimetric indicator. This test enables us to examine the recycled media for any residue amount of polymers. Below is a summary of the test which relies on a proper titration of Poly Vinyl-Sulfuric acid Potassium (PVSAK) into a sample that had been labeled with Toluidine Blue O (TBO).

First, to make a calibration curve, 125-250 mL medium from a to-be-tested pond is filtered using a 0.22 micron filter. A series (3) of 125 mL Erlenmeyer flasks are filled with 25 mL of cationic polymer of known concentrations (1, 2.5 and 5 mg/L Ashland 859BS or Nalco GR-505) in the filtered media. Each flask is then filled with an additional 25 mL of DiH2O for a total volume of 50 mL solution. Each flask is placed on a stir plate with a stir bar. In each flask, 300 uL of TBO solution is added. The solution appears blue due to the presence of cationic polymers.

From the 50 mL blue solution, an initial 200 uL (0.2 mL) solution is taken and placed in a 96 well plate. The volume of the burette is marked and associated with this well. If a burette is not available, a P200 micropipette may be used. The blue colored sample is then titrated with 0.002 PVSAK solution by adding 0.2 mL at a time from a burette or pipette until the color changed from light blue to bluish purple. For each 0.2 mL that is titrated, a 200 uL sample of the solution is taken and placed in a 96 well plate. The volume associated with each sample is also marked.

The water sample is then titrated until it turned from light blue to bluish purple. The titration is done well past the color change to be sure the endpoint appeared on the 96 well plate. Once the titration is completed, the 96 well plate with all of the titration samples is placed into the spectrophotometer. The plate is then scanned at 630 nm, and a graph of the OD results against the volume (mL) of titrant added (PVSAK) is created. FIG. 32 shows a single data series at one known concentration.

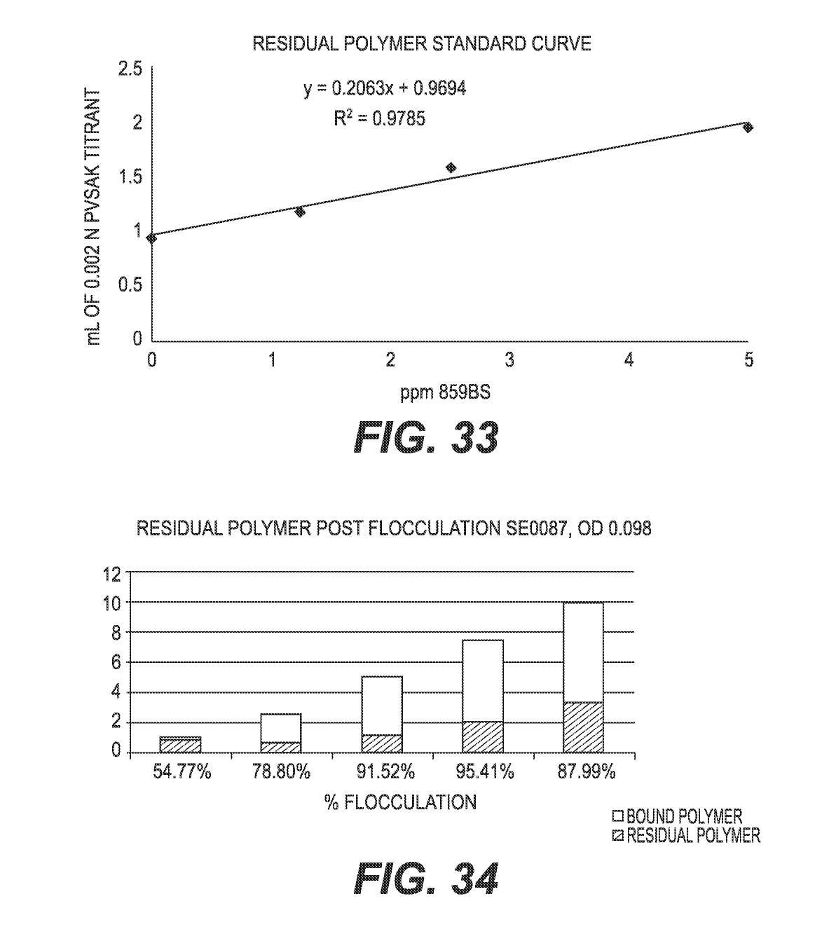

Using the PVSAK inflection point (first maximum of the first derivative shown on the auto calculated graph), the exact amount of acid required to titrate the cationic polymer is determined. Each of the known samples then has an associated volume of PVSAK with a given amount of polymer (1, 2.5, 5 mg/L). Accordingly, a calibration curve is created (FIG. 33).

### Residue Polymer Quantification and Treatment

During flocculation, polymer binds algae but a certain percentage of the polymer remains in solution. Using the above polymer quantification test, it is observed that higher polymer dosing led to an increase in the polymer that is bound to algae as well as an increase in the polymer remained in the solution (FIG. 34). Meanwhile, flocculation efficiency does not increase and in fact decreases as polymer dosing reaches certain level (FIG. 34).

Residual polymers remaining in a solution are identified and dealt with because they may have negative effects when being recycled back to the pond. Bleaching procedures are developed to identify and treat the amount of residual polymers using the polymer quantification tests mentioned above.

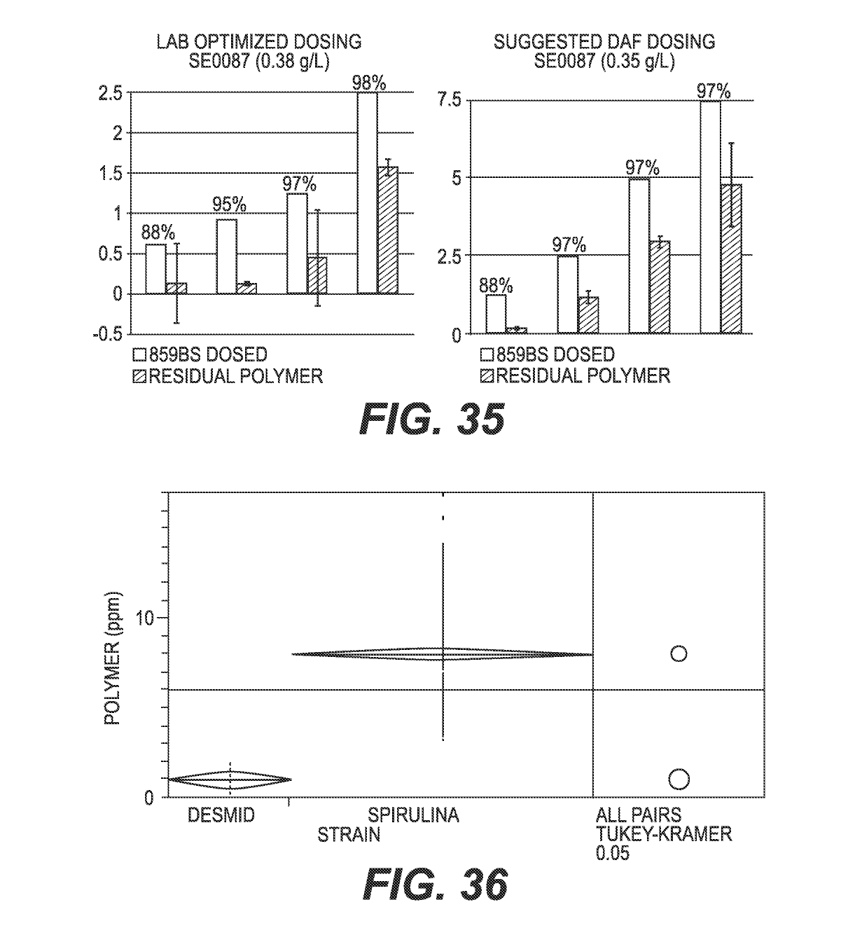

Quantification of residual polymer 859BS is performed in both laboratory tests and the DAF process. High flocculation efficiency with low level of residual polymer is achieved in both instances (FIG. 35). As polymer dosing increases, a fairly linear response of residual polymer loading is observed. In rare cases there is a decrease in residual polymer loading but this may be associated with the quantification assay.

When the residual polymer level is very low, treatment is not needed before recycling the media coming off the DAF process. The recycled media are allowed to go straight back into the pond. Based on our observation, it appears that flocculation with polymer 859BS can achieve a harvesting/dewatering condition where 90% of algae are harvested, all of the polymer is bound to the algae, and the recycled media (with 10% algae) are returned to the pond with no need for polymer removal.

### Example 7

### In-Field Large-Scale Pilot Harvest in Pond 16

An algae harvesting process can concentrate algae from ˜0.5 ppt to ˜100 ppt in an efficient and economic manner. An in-field large-scale pilot harvest process is designed and implemented based on the data collected from the in-lab and pilot-scale experimentation as well as engineering feasibility and cost studies. This in-field harvest process uses chemical flocculation followed by dissolved air flotation, and manages to concentrate algae to at least 4% solids. Further steps, e.g., a decanting centrifuge, may be taken to concentrate harvested algae slurry to optimize the solids content for downstream lipid extraction. Water stream removed from the algae is efficiently recycled. Residual chemical polymer in the recycled water stream is closely monitored for compatibility with returning to the cultivation process. If necessary, the recycled stream is treated to ensure that it does not negatively impact further cultivation process. Based on in-field pilot harvest process, it is determined that an optimal dosing rate of 96 ml/min (6 mg/L) of 0.25% polymer solution is recommended for a 95% DAF efficiency. This efficiency calculation is based on OD measurements at an operating feed rate of 10 Gallon Per Minute (GPM) algal culture feed. The Small DAF process is tested using algal culture with a starting OD measurement at 750 nm or 560 nm ranging from 0.2 to 0.85. Further, in order to operate the Small DAF at a maximum feed rate of 10 GPM, the polymer mixing station is modified to achieve an appropriate residence time of eight minutes.

### Example 9

### Analysis of Polymer Usage, Turbidity Efficiency, and TDS Level for Harvesting Scenedesmus and Spirulina

Jar tests are performed to determine the dosing of BS859 that effectively flocculates strains of Scenedesmus and Spirulina for air-assisted flotation and harvesting. Seventy two and one hundred eighty harvests are carried out for Scenedesmus and Spirulina, respectively. One-way ANOVA is used to analyze the polymer usage, turbidity efficiency, and TDS level for harvesting Scenedesmus and Spirulina. Significant differences are observed in all three aspects.

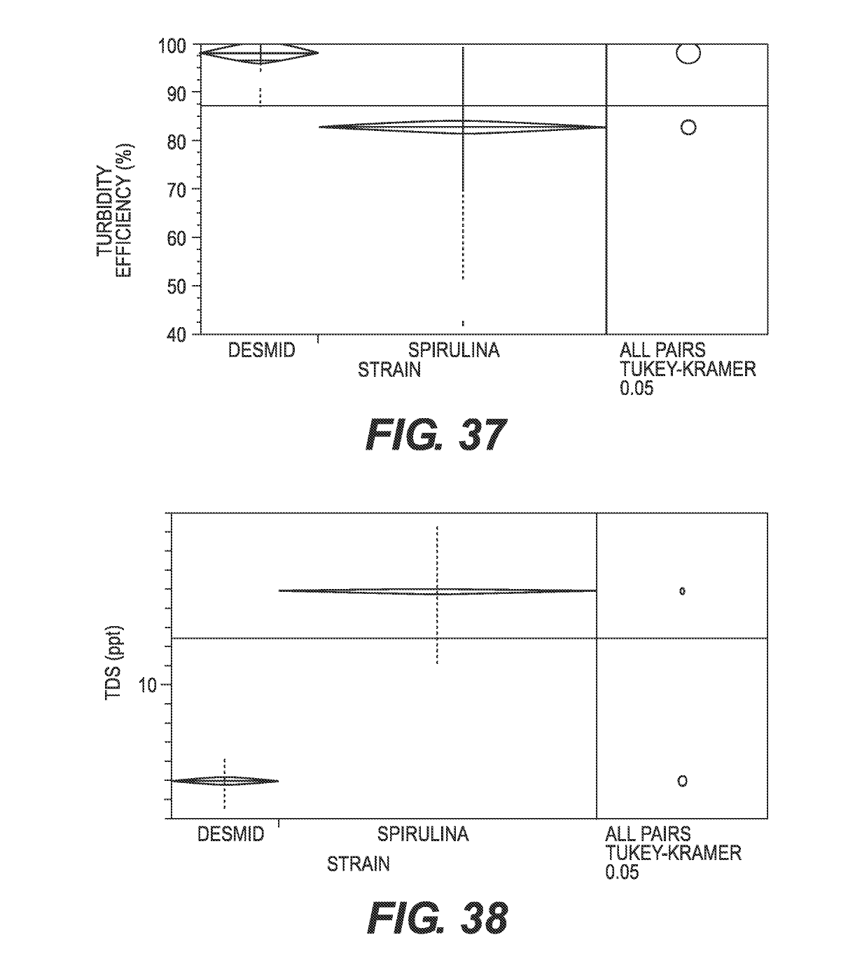

Scenedesmus and Spirulina can be effectively flocculated by an average polymer dosing of 1.04 ppm and 8.04 ppm with a standard error of 0.28 and 0.17, respectively (FIG. 36). Culture turbidity is measured before and after flocculation. The efficiency of reducing turbidity by polymer flocculation (turbidity efficiency) is calculated. On average, a turbidity efficiency of 98.5% and 83.0% is achieved with a standard error of 1.2% and 0.7% for Scenedesmus and Spirulina, respectively (FIG. 37). The average TDS level for Scenedesmus and Spirulina is 5.0 and 14.9 ppt with a standard error of 0.1 and 0.06, respectively (FIG. 38).

Lastly, the centrifuge is cleaned using a CIP solution. Manual discharge button is pressed every 5 minutes to clear solids process. CIP runs for 30 minutes or until operator determines that the rinse water is clear. The centrifuge is then shut down. The supernatant return line is also disconnected and flushed with city water. The flushed city water may be recycled to the pond harvested.

### Example 12

### Preparation and Calibration of Stock Solutions of a Dry Polymer Using a 180 Gallon Make-Down System

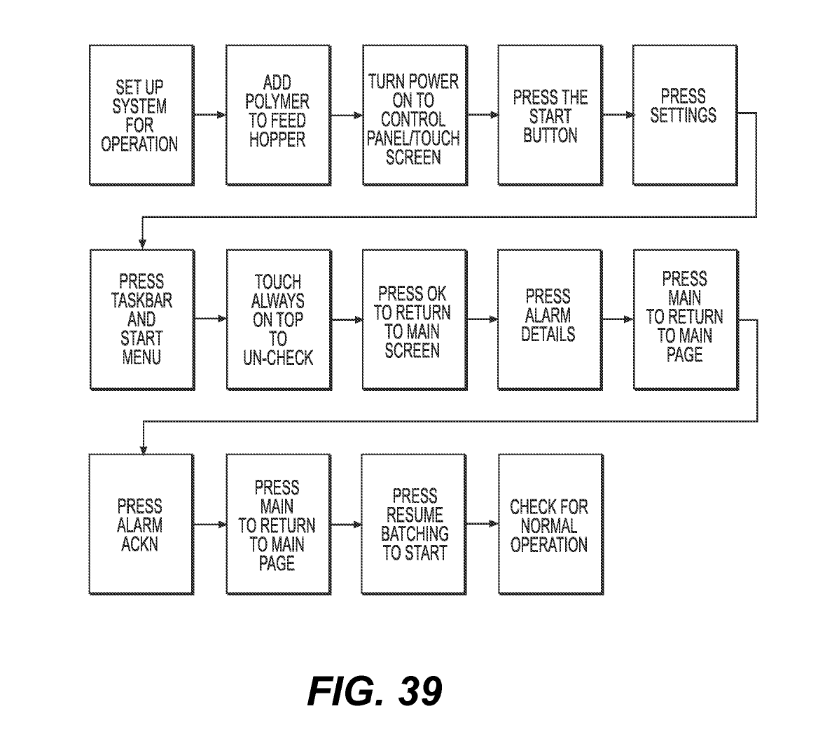

A dry polymer make-down system is used to make a 180 gallon batch of polymer stock solution for biomass flocculation within the DAF system. Below is a description of procedures for the preparation and calibration of stock solutions of a dry polymer using a 180 gallon make-down system (FIG. 39). First, a 180 gallon make-down system is set up by configuring various valves as appropriate. Dry polymer is then added to a hopper feed bin. Next, the make-down system is set to auto mode and then started. Lastly, the dry polymer stock solution is calibrated. The polymer hopper is filled with dry polymer to cover the low level sensor. The feed funnel is removed. Subsequently, the dry feeder is started, and will run for 60 seconds and then stop. The full container is weighed and time in minutes is recorded on the system setting screen.

### Example 13

### Using Jar-Tests to Determine Optimal Polymer Dosing for Biomass Harvesting by a DAF Unit

Jar-tests are used to determine optimal polymer dosing for biomass harvesting by a DAF unit. Jar tests present a lab scale simulation of culture mixing and residence time observed in a DAF harvest system. Below is a description of procedures for using jar-tests to determine optimal polymer dosing for biomass harvesting by a DAF unit (FIG. 41). First, a 0.25% Ashland 859BS polymer stock solution is prepared. 500 ml of sample culture is poured into four jars. Mixers are placed at the top of the jars so that the lock tab sits against the jar wall. The mixer is turned to 300 rpm. Polymer is added to the culture (Table 27). The culture is allowed to mix for 30 seconds. The mixer is then stopped to allow the culture to flocculate and settle. The efficiency of algae polymer flocculation is determined visually or measured using a turbidimeter. Culture samples are collected using jar valves or directly from the culture using a pipette. Both initial and final culture OD values are measured at 560 or 750 nm (depending on strain). Harvesting efficiency is calculated using the measured OD values and the following formula: Efficiency percentage=(OD initial−OD final)/OD initial×100.

TABLE 27
Examples of a polymer concentration gradient used in jar tests.

| Jar # | mL 0.25% Polymer | mg/L Polymer | |
|---|---|---|---|
| 1 | 500 uL | 2.5 | mg/L |
| 2 | 1000 uL | 5 | mg/L |
| 3 | 1500 uL | 7.5 | mg/L |
| 4 | 2000 uL | 10 | mg/L |

### Example 14

### Operation of a 40 Gpm WWW DAF System to Harvest Algae

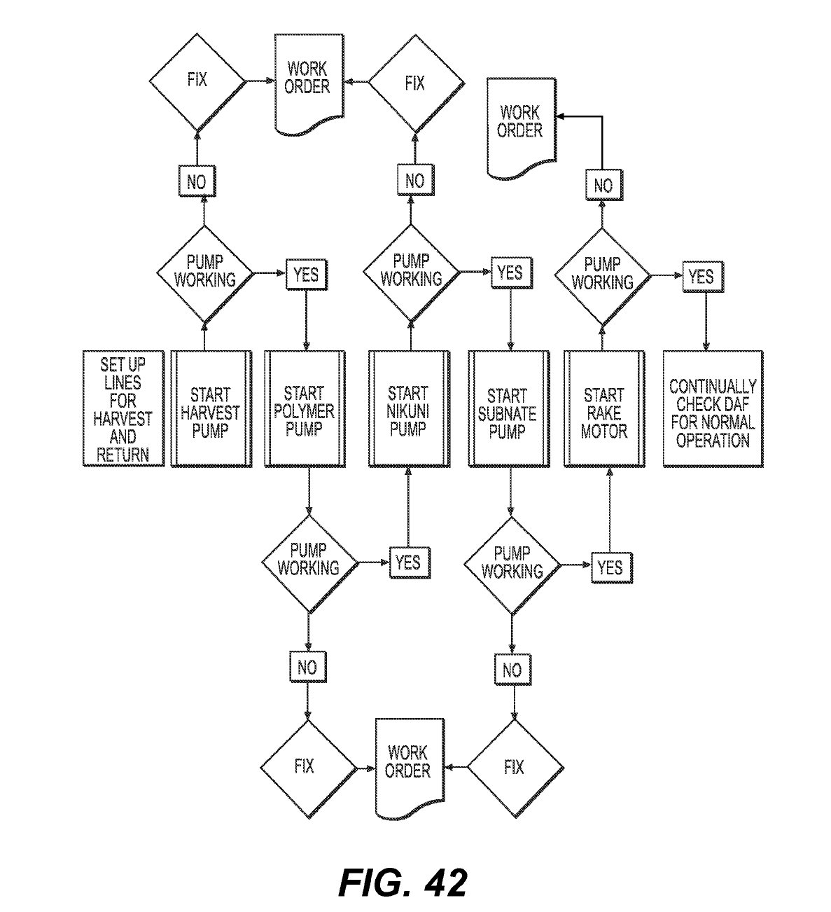

A 40 gpm WWW DAF system is used to harvest biomass from ponds. Below is a description of the procedures for using a 40 gpm WWW DAF system to harvest algae (FIG. 42). First, power for the 40-GPM WWW DAF Control Panel is turned on followed by switching on power for Mixer-1 (or Mixer-2), a Nikuni Pump (Nikuni 1), and a Rake Motor (RM1). E-Stops at DAF area Control Panel for Return Pump, Harvest Pump, DAF Feed Pump and Subnatant Pump are set at an OFF opposition. Harvest lines from pond sump are connected to the Harvest Pump. Subnatant return pump lines are set up so that subnatant is returned back to the pond. Various valves of the DAF system are then set in an appropriate configuration. Polymer injection port is opened. Polymer pump is set to prime for 30 seconds.

Next, the DAF Feed Pump is turned on followed by switching on the polymer pump once the flow of algae has started. Aeration Pump is switched to HAND after the DAF unit is filled above the internal laterals. Nikuni Pump air inlet pressure gauge is set between 1-3 psi. Air inlet to Nikuni Pump is set at 8 SCFH. Subnatant Saturation Tank pressure is set between 50-60 psi. Water is allowed to flow to Subnatant Return chamber. Power of DAF Subnatant Pump is turned on. DAF float depth and DAF Subnatant clarity are monitored. Polymer injection rate is adjusted if necessary. Rake switch is turned to HAND at DAF Control Panel. Product outlet valve (PV1) is opened to collect DAF product.

Lastly, the DAF system is cleaned and shut down. The DAF feed pump is turned off followed by polymer pump. The valve at pond sump is closed. Hose is disconnected from pond sump and then connected to city water source. Power of the DAF feed pump is turned back on. Lines are flushed with city water until leaf trap is visibly clear. Then the power of DAF feed pump is turned off. A DAF drain valve (DV2-3) is opened. The aeration pump is turned off to allow the flushed water to drain back to the pond until the water level reaches the internal laterals. Interior of the DAF unit is sprayed with spray nozzle to remove flocked algae. The DAF unit is allowed to drain completely before the power of the DAF subnatant pump is turned off. The subnatant return line is then disconnected from the subnatant pump followed by flushing the subnatant return line until it becomes clear.

### Example 15

### Operation of a Westech Dissolved Air Flotation (DAF) Unit to Harvest Algal Biomass from a Pond

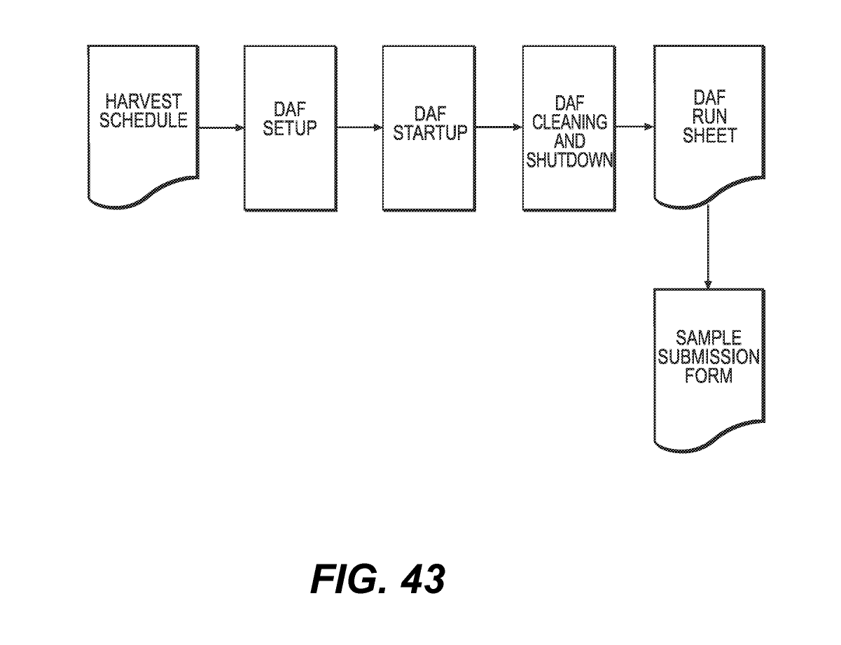

A Westech Dissolved Air Flotation (DAF) unit is used to harvest biomass from ponds. Below is a description of the operating procedures (FIG. 43). First, harvest schedule input is used to determine harvest volume. Pre-harvest samples are collected with pond depth measured. Jar tests are used to determine the harvest polymer dosing rate (mg/L) and polymer volume (gallons) needed. Polymer Dosing rate (mL/min) is determined using the following:

$$Rate = Dose ( \frac{mg}{L} ) \div 2.5 \frac{g}{L} \times Lpm DAF Inflow$$

Polymer Volume for harvest (gallons) is determined using the following:

$$Harvest Time ( min . ) \times Dose Rate ( \frac{mL}{min} ) \div 3785 \frac{mL}{gal} .$$

Second, Main power of the Westech DAF operating panel is switched on. Air compressor is switched on (for non-spirulina strains). Harvest lines are set up from pond sump, to harvest pump, and to DAF. Subnatant return lines are also set up so that subnatant is returned back to the pond harvested. Various valves of the DAF system are then set in an appropriate configuration. Polymer pump is then set at an appropriate speed and prime. Product tote is weighed for tare value.

Next, the DAF tank is filled with water (Not required for spirulina harvest). A recycle pump is switched on (Not required for spirulina harvest). The DAF feed pump is started followed by the polymer pump. Product pump P-320 is switched on with its speed set to 500%. The drive unit is then switched on followed by the subnatant return pump. Samples are collected with harvest data recorded.

Lastly, the DAF system is cleared and shut down. Lines from the pond to the DAF are rinsed with city water. The drained rinsing water is returned back to the pond harvested. Totes with harvested product are weighed.

### Example 16

### Operation of a Decanter to Further Dewater a Biomass Slurry Harvested by a DAF Process

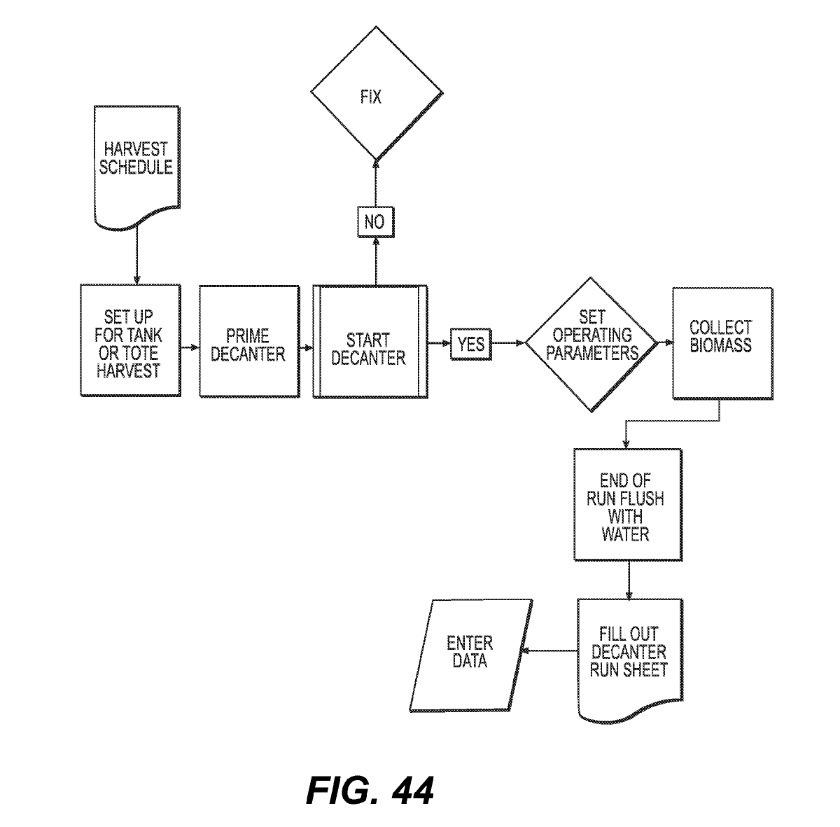

A decanter is used to further dewater a biomass slurry harvested by a DAF process. Below is a description of the operating procedures (FIG. 44). First, greasing is applied to main shaft bearings before each use. The bowl of the decanter is primed by undoing the tri-clamp at the feed-in elbow on the decanter. A water hose is placed in the elbow and the bowl is filled with water until water begins to flow out of the product hopper. A bucket is placed under product hopper.

Next, a centrate line (Effluent) is connected to EVAP drain or tank followed by opening a gate valve on EVAP drain or tank valve. The decanter is started and allowed to spin up to 5200 rpm. Subsequently, a feed pump is started and set at 50% speed which may be adjusted during a run. Back pressure valve is adjusted to clear out centrate (effluent). Differential (Dn) is adjusted to adjust scroll speed. A lower Dn value can lead to a slower process and a thicker product with more loss to centrate. A higher Dn value can result in less loss to centrate, but the product may be thinner or have more moisture. After product is collected, water is applied to rinse the decanter system until centrate (effluent) becomes clear before the decanter is stopped.

### Example 17

### Sample Collection During a Harvest of Algae from a Pond by a DAF Process

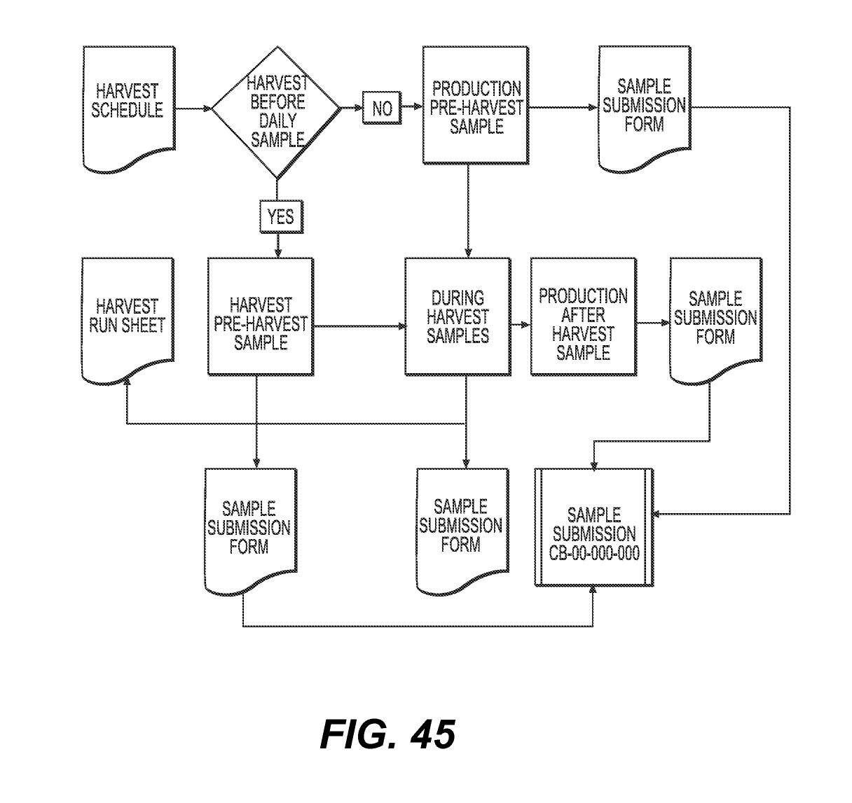

Samples are collected and analyzed prior to, during, and after a DAF process. FIG. 45 describes procedures for sample collection associated with a DAF process. A to-be-harvested pond is sampled and analyzed prior to and after a DAF process to monitor harvesting efficiency. For sample collections prior to a DAF process, the to-be-harvested pond is mixed using a paddle wheel for at least 30 min. Samples are measured for pre-harvest dry weight (Pre Harvest DW). Paddle wheel is then turned off for at least 5 min to record pond depth. After a DAF process, samples are collected at two time points: late morning and late afternoon, and measured for post-harvest dry weight (Post Harvest DW). Pond depth is also recorded.

Samples are collected from an input stream prior to the injection of polymers (input), output stream (subnatant), and harvested algae slurry (product). Samples are taken during a DAF process at three time points: T=0 (start of harvest), T=½ (time point when half of algae culture is processed), T=1 (end point). Samples collected from the input stream and subnatant are measured for turbidity (a total of 6 samples: Input T=0, T=½, T=1 and Subnatant T=0, T=½, T=1). Dry weight data are collected from the input, subnatant, and product samples. Input dry weight (Input DW) is measured from a mixed input sample which consists of 40 mL from input T=0, 40 mL from input T=½, and 40 mL from input T=1. Subnatant dry weight (Subnatant DW) is measured from a mixed subnatant sample which consists of 40 mL from subnatant T=0, 40 mL from subnatant T=½, and 40 mL from subnatant T=1. Product dry weight (Product DW) is measured from a mixed product sample which consists of 40 mL from product T=0, 40 mL from product T=½, and 40 mL from product T=1.

### Example 18

### Moisture Analysis, Packaging and Storage of Harvested Algal Product

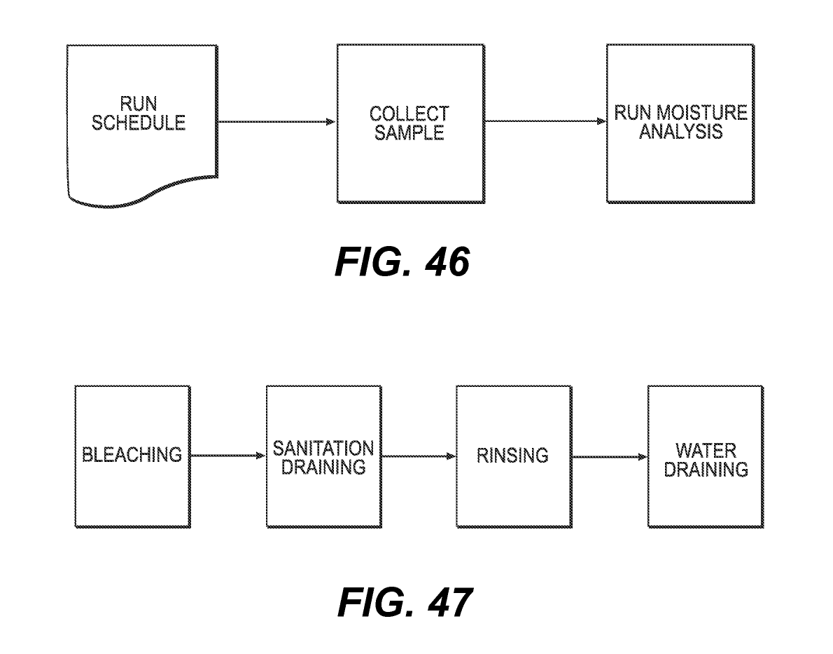

Harvested algal product is analyzed using a Mettler Toledo HB-43-S Halogen Moisture Content Analyzer to evaluate water and solids content (FIG. 46). Harvested algal product is then packaged and stored using either 5 gallon buckets or 250 gallon totes.

### Example 19

### Sanitization of a DAF Unit

A Westech Dissolved Air Flotation (DAF) unit (DAF-2) is sanitized using a solution containing bleach. The sanitization solution is recirculated inside the main DAF chamber in order to kill any remaining bacteria and other contaminants after a harvest. The DAF unit is then rinsed with city water. Below is a summary of the procedures (FIG. 47). First, the DAF unit is set up for sanitization with various valves at an appropriate configuration. A 2-inch hose is connected between city water supply and bottom solids Outlet. Next, bottom solids out valve (DDU-12) is opened followed by turning on city water. About 4 gallons of a 6% solution of germicidal bleach is added while main DAF chamber is being filled with city water. Main DAF chamber is allowed to be filled to the top of subnatant outlet chamber weir gate before city water is turned off. City water and bleach solution are allowed to recirculate for 5 minutes before recycle pump (P-410) is turned off. City water and bleach solution are left in main DAF chamber for a minimum contact time of 30 minutes. Next, the sanitization solution is drained. Following draining of the sanitization solution, the DAF unit is rinsed with city water.

1-81. (canceled)

82. A method to harvest a non-vascular photosynthetic organism (NVPO) from an aqueous culture, comprising, mixing an effective amount of a cationic organic polymer flocculant with said aqueous culture of said NVPO to form floes of said NVPO; and collecting said flocs of said NVPO, wherein said aqueous culture has a total dissolved solids (TDS) of at least 1500 milligram per liter (mg/L).

83. The method of claim 82, wherein said floes are collected by sedimentation.

84. The method of claim 82, further comprising, introducing dissolved air into said mixed aqueous culture; and collecting said flocs by dissolved air floatation.

85. The method of claim 82, wherein said aqueous culture is an open pond culture.

86. The method of claim 82, wherein said aqueous culture comprises a TDS selected from the group consisting of between 1500 and 35000, between 5000 and 35000, and between 15000 and 35000 mg/L.

87. The method of claim 82, wherein said NVPO in said aqueous culture is at a concentration between 0.001% and 0.2%, and said effective amount of said cationic organic polymer flocculant is less than 10 parts per million (ppm).

88. The method of claim 82, wherein said method produces a biomass slurry comprising said NVPO and said cationic organic polymer flocculant, said NVPO in said harvested biomass slurry is at a concentration of at least 0.2%, and the ratio between the weight of said cationic organic polymer flocculant and the Ash-Free Dry Weight (AFDW) of said NVPO in said harvested biomass slurry is at most 1.5%.

89. The method of claim 82, wherein said method produces a aqueous medium comprising said NV PO at a concentration between 0.0001% and 0.02%, and said cationic organic polymer flocculant at a concentration of at most 1 parts per million (ppm).

90. The method of claim 82, wherein said cationic organic polymer flocculant has a stock concentration selected from the group consisting of 0.1%, 0.25%, 0.5%, 0.75%, and 1%.

91. The method of claim 82, wherein said cationic organic polymer flocculant has no significant impacts on the group consisting of growth of said NVPO, pH of said aqueous culture, nitrate level of said aqueous culture, and phosphate level of said aqueous culture.

92. The method of claim 82, wherein said flocs of said NVPO have an average diameter selected from the group consisting of at least 0.05, 0.1, 0.2, 0.3, 0.5, 1, 2, 5, 10, 20, 30, 40, 50, and 60 millimeters.

93. The method of claim 82, wherein at least 90% of said NVPO in said aqueous culture is harvested through said method.

94. The method of claim 82, wherein said method achieves a harvest volume of less than 30% of the volume of said aqueous solution.

95. The method of claim 82, wherein said NVPO is from a genus selected from the group consisting of Chlamydomonas, Nannochloropsis, Scenedesmus, Desmodesmus, Tetraselmis, and Arthrospira.

96. The method of claim 82, wherein said method further comprises mixing an effective amount of an anionic polymer flocculant with said aqueous culture of said NVPO.

97. A method to harvest a non-vascular photosynthetic organism (NVPO) from an open pond aqueous culture by dissolved air flotation, comprising mixing an effective amount of a cationic organic polymer flocculant with said open pond aqueous culture to form flocs of said NVPO; introducing dissolved air into said mixed aqueous culture; and collecting said floes of said NVPO, wherein said aqueous culture has a total dissolved solids (TDS) of at least 1500 milligram per liter (mg/L).

98. The method of claim 97, wherein said aqueous culture comprises a TDS selected from the group consisting of between 1500 and 35000, between 5000 and 35000, and between 15000 and 35000 mg/L.

99. The method of claim 97, wherein said NVPO in said aqueous culture is at a concentration between 0.001% and 0.2%, and said effective amount of said polymer flocculant is less than 10 parts per million (ppm).

100. The method of claim 97, wherein said polymer flocculant has no significant impacts on the group consisting of growth of said NVPO, pH of said aqueous culture, nitrate level of said aqueous culture, and phosphate level of said aqueous culture.

101. The method of claim 97, wherein said NVPO is from a genus selected from the group consisting of Nannochloropsis, Scenedesmus, Desmodesmus, Tetraselmis, and Arthrospira.
